SEQUENCE LISTING (129 sequences)

SEQ ID NO: 1 - DNA, 69 bp, Artificial
  p7.5tk Promoter
ggccaaaaattgaaaaactagatctatttattgcacgcggccgccatgggcccggccgccaacggcgga

SEQ ID NO: 2 - protein, 8 aa, Artificial
  First 8 Amino Acids of Vaccina Virus Thymidine Kinase
MGPAANGG

SEQ ID NO: 3 - protein, 15 aa, Artificial
  Linker
GGGGSGGGGSGGGGS

SEQ ID NO: 4 - protein, 15 aa, Artificial
  Linker
ESGRSGGGGSGGGGS

SEQ ID NO: 5 - protein, 14 aa, Artificial
  Linker
EGKSSGSGSESKST

SEQ ID NO: 6 - protein, 15 aa, Artificial
  Linker
EGKSSGSGSESKSTQ

SEQ ID NO: 7 - protein, 14 aa, Artificial
  Linker
EGKSSGSGSESKVD

SEQ ID NO: 8 - protein, 14 aa, Artificial
  Linker
GSTSGSGKSSEGKG

SEQ ID NO: 9 - protein, 18 aa, Artificial
  Linker
KESGSVSSEQLAQFRSLD

SEQ ID NO: 10 - protein, 16 aa, Artificial
  Linker
ESGSVSSEELAFRSLD

SEQ ID NO: 11 - DNA, 1555 bp, Artificial
  pVHE
ggccaaaaattgaaaaactagatctatttattgcacgcggccgcaaaccatgggatggagctgtatcatcctcttcttggtagcaacagctacaggcgcgcatatggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcagcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgactagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcactgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatgagtcgac

SEQ ID NO: 12 - DNA, 446 bp, Artificial
  pVKE
ggccaaaaattgaaaaactagatctatttattgcacgcggccgcccatgggatggagctgtatcatcctcttcttggtagcaacagctacaggcgtgcacttgactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgac

SEQ ID NO: 13 - DNA, 455 bp, Artificial
  pVLE
ggccaaaaattgaaaaactagatctatttattgcacgcggccgcccatgggatggagctgtatcatcctcttcttggtagcaacagctacaggcgtgcacttgactcgagaagcttaccgtcctacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgac

SEQ ID NO: 14 - DNA, 21 bp, Artificial
  Primer CH4(F)
ctctcccgcggacgtcttcgt

SEQ ID NO: 15 - DNA, 30 bp, Artificial
  Primer CH4(R2)
aatagtggtgatatatttcaccttgaacaa

SEQ ID NO: 16 - DNA, 30 bp, Artificial
  Primer FAS(F3)
ttgttcaaggtgaaagtgaagagaaaggaa

SEQ ID NO: 17 - DNA, 33 bp, Artificial
  Primer FAS(R)
acgcgtcgacctagaccaagctttggatttcat

SEQ ID NO: 18 - DNA, 49 bp, Artificial
  Primer pstmutF
gtcgaataaagtgaacaataattaattctgcagtgtcatcatggcggcc

SEQ ID NO: 19 - DNA, 49 bp, Artificial
  Primer pstmutR
ggccgccatgatgacactgcagaattaattattgttcactttattcgac

SEQ ID NO: 20 - DNA, 72 bp, Artificial
  H5-PN-S
gaaaaaatgaaaataaatacaaaggttcttgagggttgtgttaaattgaaagcgagaaataatcataaattc

SEQ ID NO: 21 - DNA, 80 bp, Artificial
  H5-PN-AS
catggaatttatgattatttctcgctttcaatttaacacaaccctcaagaacctttgtatttattttcattttttctgca

SEQ ID NO: 22 - DNA, 29 bp, Artificial
  Primer C-gamma1F
attaggatccggtcaccgtctcctcagcc

SEQ ID NO: 23 - DNA, 31 bp, Artificial
  Primer C-gamma1R
attagtcgacctaggccccctgtccgatcat

SEQ ID NO: 24 - DNA, 27 bp, Artificial
  Primer BstEIImutF
cagcgtggtgactgtgccctccagcag

SEQ ID NO: 25 - DNA, 27 bp, Artificial
  Primer BstEIImutR
ctgctggagggcacagtcaccacgctg

SEQ ID NO: 26 - DNA, 5103 bp, Artificial
  VLE H5
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctgcagaaaaaatgaaaataaatacaaaggttcttgagggttgtgttaaattgaaagcgagaaataatcataaattccatgggatggagctgtatcatcctcttcttggtagcaacagctacaggcgtgcacttgactcgagaagcttaccgtcctacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgacctcgatcgagggggggcctaactaactaattttgtttttgtgggcccggccgccaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgatcgagttaccaaatcagacgctgtaaattatgaaaaaaagatgtactaccttaataagatcgtccgtaacaaagtagttgtttgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcatgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 27 - protein, 110 aa, Artificial
  cLambda Constant Domain
TVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 5094 bp, Artificial
  VKE H5
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctgcagaaaaaatgaaaataaatacaaaggttcttgagggttgtgttaaattgaaagcgagaaataatcataaattccatgggatggagctgtatcatcctcttcttggtagcaacagctacaggcgtgcacttgactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgacctcgatcgagggggggcctaactaactaattttgtttttgtgggcccggccgccaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgatcgagttaccaaatcagacgctgtaaattatgaaaaaaagatgtactaccttaataagatcgtccgtaacaaagtagttgtttgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcatgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 29 - protein, 106 aa, Artificial
  cKappa Constant Domain
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 5972 bp, Artificial
  VHE H5MBG
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctgcagaaaaaatgaaaataaatacaaaggttcttgagggttgtgttaaattgaaagcgagaaataatcataaattccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccggagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctaggtcgacctcgatcgagggggggcctaactaactaattttgtttttgtgggcccggccgccaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgatcgagttaccaaatcagacgctgtaaattatgaaaaaaagatgtactaccttaataagatcgtccgtaacaaagtagttgtttgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcatgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 31 - protein, 402 aa, Artificial
  Membrane-bound IgG1 Constant Domain
VSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 32 - DNA, 6197 bp, Artificial
  VHE H5MBMu
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctgcagaaaaaatgaaaataaatacaaaggttcttgagggttgtgttaaattgaaagcgagaaataatcataaattccatgggatggagctgtatcatcctcttcttggtagcaacagctacaggcgcgcatatggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgccgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaagtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcactgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatgagtcgacctcgatcgagggggggcctaactaactaattttgtttttgtgggcccggccgccaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgatcgagttaccaaatcagacgctgtaaattatgaaaaaaagatgtactaccttaataagatcgtccgtaacaaagtagttgtttgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcatgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 33 - protein, 479 aa, Artificial
  Membrane-bound IgM Constant Domain
VTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTEGEVSADEEGFENLWATASTFIVLFLLSLFYSTTVTLFKVK

SEQ ID NO: 34 - DNA, 5766 bp, Artificial
  VHE H5GS
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagcctccaagggtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattcctgcagaaaaaatgaaaataaatacaaaggttcttgagggttgtgttaaattgaaagcgagaaataatcataaattccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcgacctcgatcgagggggggcctaactaactaattttgtttttgtgggcccggccgccaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgatcgagttaccaaatcagacgctgtaaattatgaaaaaaagatgtactaccttaataagatcgtccgtaacaaagtagttgtttgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcatgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 35 - protein, 335 aa, Artificial
  IgG Secreted Constant Domain
VTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 11 bp, Artificial
  VH Primer
gcgcgcactcc

SEQ ID NO: 37 - DNA, 38 bp, Artificial
  Primer VH1
ttttgcgcgcactcccaggtgcagctggtgcagtctgg

SEQ ID NO: 38 - DNA, 39 bp, Artificial
  Primer VH2
aatatgcgcgcactcccaggtcaccttgaaggagtctgg

SEQ ID NO: 39 - DNA, 38 bp, Artificial
  Primer VH3
ttttgcgcgcactccgaggtgcagctggtggagtctgg

SEQ ID NO: 40 - DNA, 38 bp, Artificial
  Primer VH4
ttttgcgcgcactcccaggtgcagctgcaggagtcggg

SEQ ID NO: 41 - DNA, 38 bp, Artificial
  Primer VH5
aatatgcgcgcactccgaggtgcagctggtgcagtctg

SEQ ID NO: 42 - DNA, 27 bp, Artificial
  Primer JH1
gacggtgaccagggtgccctggcccca

SEQ ID NO: 43 - DNA, 27 bp, Artificial
  Primer JH2
gacggtgaccagggtgccacggcccca

SEQ ID NO: 44 - DNA, 27 bp, Artificial
  Primer JH3
gacggtgaccattgtcccttggcccca

SEQ ID NO: 45 - DNA, 27 bp, Artificial
  Primer JH4/5
gacggtgaccagggttccctggcccca

SEQ ID NO: 46 - DNA, 27 bp, Artificial
  Primer JH6
gacggtgaccgtggtcccttggcccca

SEQ ID NO: 47 - DNA, 35 bp, Artificial
  Primer V-Kappa 1
tttgtgcactccgacatccagatgacccagtctcc

SEQ ID NO: 48 - DNA, 35 bp, Artificial
  Primer V-Kappa 2
tttgtgcactccgatgttgtgatgactcagtctcc

SEQ ID NO: 49 - DNA, 35 bp, Artificial
  Primer V-Kappa 3
tttgtgcactccgaaattgtgttgacgcagtctcc

SEQ ID NO: 50 - DNA, 35 bp, Artificial
  Primer V-Kappa 4
tttgtgcactccgacatcgtgatgacccagtctcc

SEQ ID NO: 51 - DNA, 35 bp, Artificial
  Primer V-Kappa 5
tttgtgcactccgaaacgacactcacgcagtctcc

SEQ ID NO: 52 - DNA, 35 bp, Artificial
  Primer V-Kappa 6
tttgtgcactccgaaattgtgctgactcagtctcc

SEQ ID NO: 53 - DNA, 27 bp, Artificial
  Primer J-Kappa 1
gatctcgagcttggtcccttggccgaa

SEQ ID NO: 54 - DNA, 27 bp, Artificial
  Primer J-Kappa 2
gatctcgagcttggtcccctggccaaa

SEQ ID NO: 55 - DNA, 27 bp, Artificial
  Primer J-Kappa 3
gatctcgagtttggtcccagggccgaa

SEQ ID NO: 56 - DNA, 27 bp, Artificial
  Primer J-Kappa 4
gatctcgagcttggtccctccgccgaa

SEQ ID NO: 57 - DNA, 27 bp, Artificial
  Primer J-Kappa 5
aatctcgagtcgtgtcccttggccgaa

SEQ ID NO: 58 - DNA, 35 bp, Artificial
  Primer V-Lambda 1
tttgtgcactcccagtctgtgttgacgcagccgcc

SEQ ID NO: 59 - DNA, 35 bp, Artificial
  Primer V-Lambda 2
tttgtgcactcccagtctgccctgactcagcctgc

SEQ ID NO: 60 - DNA, 35 bp, Artificial
  Primer V-Lambda 3A
tttgtgcactcctcctatgtgctgactcagccacc

SEQ ID NO: 61 - DNA, 35 bp, Artificial
  Primer V-Lambda 3B
tttgtgcactcctcttctgagctgactcaggaccc

SEQ ID NO: 62 - DNA, 35 bp, Artificial
  Primer V-Lambda 4
tttgtgcactcccacgttatactgactcaaccgcc

SEQ ID NO: 63 - DNA, 35 bp, Artificial
  Primer V-Lambda 5
tttgtgcactcccaggctgtgctcactcagccgtc

SEQ ID NO: 64 - DNA, 35 bp, Artificial
  Primer V-Lambda 6
tttgtgcactccaattttatgctgactcagcccca

SEQ ID NO: 65 - DNA, 35 bp, Artificial
  Primer V-Lambda 7
tttgtgcactcccaggctgtggtgactcaggagcc

SEQ ID NO: 66 - DNA, 27 bp, Artificial
  Primer J-Lambda 1
ggtaagcttggtcccagttccgaagac

SEQ ID NO: 67 - DNA, 25 bp, Artificial
  Primer J-Lambda 2/3
ggtaagcttggtccctccgccgaat

SEQ ID NO: 68 - DNA, 39 bp, Artificial
  Primer VH1a
aatatgcgcgcactcccaggtgcagctggtgcagtctgg

SEQ ID NO: 69 - DNA, 39 bp, Artificial; identical to SEQ ID NO: 38
  Primer VH2a

SEQ ID NO: 70 - DNA, 39 bp, Artificial
  Primer VH3a
aatatgcgcgcactccgaggtgcagctggtggagtctgg

SEQ ID NO: 71 - DNA, 39 bp, Artificial
  Primer VH4a
aatatgcgcgcactcccaggtgcagctgcaggagtcggg

SEQ ID NO: 72 - DNA, 38 bp, Artificial; identical to SEQ ID NO: 41
  Primer VH5a

SEQ ID NO: 73 - DNA, 29 bp, Artificial
  Primer JH1a
gagacggtgaccagggtgccctggcccca

SEQ ID NO: 74 - DNA, 29 bp, Artificial
  Primer JH2a
gagacggtgaccagggtgccacggcccca

SEQ ID NO: 75 - DNA, 29 bp, Artificial
  Primer JH3a
gagacggtgaccattgtcccttggcccca

SEQ ID NO: 76 - DNA, 29 bp, Artificial
  Primer JH4/5a
gagacggtgaccagggttccctggcccca

SEQ ID NO: 77 - DNA, 29 bp, Artificial
  Primer JH6a
gagacggtgaccgtggtcccttggcccca

SEQ ID NO: 78 - DNA, 37 bp, Artificial
  Primer V-Kappa 1a
caggagtgcactccgacatccagatgacccagtctcc

SEQ ID NO: 79 - DNA, 37 bp, Artificial
  Primer V-Kappa 2a
caggagtgcactccgatgttgtgatgactcagtctcc

SEQ ID NO: 80 - DNA, 37 bp, Artificial
  Primer V-Kappa 3a
caggagtgcactccgaaattgtgttgacgcagtctcc

SEQ ID NO: 81 - DNA, 37 bp, Artificial
  Primer V-Kappa 4a
caggagtgcactccgacatcgtgatgacccagtctcc

SEQ ID NO: 82 - DNA, 37 bp, Artificial
  Primer V-Kappa 5a
caggagtgcactccgaaacgacactcacgcagtctcc

SEQ ID NO: 83 - DNA, 37 bp, Artificial
  Primer V-Kappa 6a
caggagtgcactccgaaattgtgctgactcagtctcc

SEQ ID NO: 84 - DNA, 29 bp, Artificial
  Primer J-Kappa 1a
ttgatctcgagcttggtcccttggccgaa

SEQ ID NO: 85 - DNA, 29 bp, Artificial
  Primer J-Kappa 2a
ttgatctcgagcttggtcccctggccaaa

SEQ ID NO: 86 - DNA, 29 bp, Artificial
  Primer J-Kappa 3a
ttgatctcgagtttggtcccagggccgaa

SEQ ID NO: 87 - DNA, 29 bp, Artificial
  Primer J-Kappa 4a
ttgatctcgagcttggtccctccgccgaa

SEQ ID NO: 88 - DNA, 29 bp, Artificial
  Primer J-Kappa 5a
ttaatctcgagtcgtgtcccttggccgaa

SEQ ID NO: 89 - DNA, 37 bp, Artificial
  Primer V-Lambda 1a
cagatgtgcactcccagtctgtgttgacgcagccgcc

SEQ ID NO: 90 - DNA, 37 bp, Artificial
  Primer V-Lambda 2a
cagatgtgcactcccagtctgccctgactcagcctgc

SEQ ID NO: 91 - DNA, 37 bp, Artificial
  Primer V-Lambda 3Aa
cagatgtgcactcctcctatgtgctgactcagccacc

SEQ ID NO: 92 - DNA, 37 bp, Artificial
  Primer V-Lambda 3Ba
cagatgtgcactcctcttctgagctgactcaggaccc

SEQ ID NO: 93 - DNA, 37 bp, Artificial
  Primer V-Lambda 4a
cagatgtgcactcccacgttatactgactcaaccgcc

SEQ ID NO: 94 - DNA, 37 bp, Artificial
  Primer V-Lambda 5a
cagatgtgcactcccaggctgtgctcactcagccgtc

SEQ ID NO: 95 - DNA, 37 bp, Artificial
  Primer V-Lambda 6a
cagatgtgcactccaattttatgctgactcagcccca

SEQ ID NO: 96 - DNA, 37 bp, Artificial
  Primer V-Lambda 7a
cagatgtgcactcccaggctgtggtgactcaggagcc

SEQ ID NO: 97 - DNA, 29 bp, Artificial
  Primer J-Lambda 1a
acggtaagcttggtcccagttccgaagac

SEQ ID NO: 98 - DNA, 29 bp, Artificial
  Primer J-Lambda 2/3a
acggtaagcttggtccctccgccgaatac

SEQ ID NO: 99 - protein, 8 aa, Artificial
  Epitope
SIINFEKL

SEQ ID NO: 100 - DNA, 21 bp, Artificial
  Primer Gus Sense
atgttacgtcctgtagaaacc

SEQ ID NO: 101 - DNA, 21 bp, Artificial
  Primer Gus Antisense
tcattgtttgcctccctgctg

SEQ ID NO: 102 - DNA, 28 bp, Artificial
  Primer NX-Gus Sense
aaagcggccgccccgggatgttacgtcc

SEQ ID NO: 103 - DNA, 29 bp, Artificial
  Primer AA-Gus Antisense
aaagggcccggcgcgcctcattgtttgcc

SEQ ID NO: 104 - DNA, 37 bp, Artificial
  Primer D4R-Sense
aaaggatccataatgaattcagtgactgtatcacacg

SEQ ID NO: 105 - DNA, 34 bp, Artificial
  Primer D4R Antisense
cttgcggccgcttaataaataaacccttgagccc

SEQ ID NO: 106 - DNA, 34 bp, Artificial
  Primer D4R Flank Sense
attgagctcttaatacttttgtcgggtaacagag

SEQ ID NO: 107 - DNA, 29 bp, Artificial
  Primer D4R Flank Antisense
ttactcgagagtgtcgcaatttggatttt

SEQ ID NO: 108 - DNA, 29 bp, Artificial
  Primer 7.5 Gus Sense
aaagaattcctttattgtcatcggccaaa

SEQ ID NO: 109 - DNA, 30 bp, Artificial
  Primer 7.5 Gus Antisense
aatctgcagtcattgtttgcctccctgctg

SEQ ID NO: 110 - DNA, 51 bp, Artificial
  p7.5/tk3.1 Insert Cassette
gcggccgcccatggatagcgtgcacttgactcgagaagcttagtagtcgac

SEQ ID NO: 111 - DNA, 22 bp, Artificial
  p7.5/tk3.1 XhoI-SalI
ctcgagaagcttagtagtcgac

SEQ ID NO: 112 - DNA, 78 bp, Artificial
  p7.5/tk3.2 Insert Cassette
ctcgagatcaaagagggtaaatcttccggatctggttccgaaggcgcgcatgcggtcaccgtctcctcatgagtcgac

SEQ ID NO: 113 - DNA, 42 bp, Artificial
  VKappa-VH Linker
gagggtaaatcttccggatctggttccgaaggcgcgcactcc

SEQ ID NO: 114 - protein, 14 aa, Artificial
  VKappa-VH Linker
EGKSSGSGSEGAHS

SEQ ID NO: 115 - DNA, 16 bp, Artificial
  p7.5/tk3.1 HindIII-SalI
aagcttagtagtcgac

SEQ ID NO: 116 - DNA, 81 bp, Artificial
  p7.5/tk3.3 Insert Cassette
aagcttaccgtcctagagggtaaatcttccggatctggttccgaaggcgcgcatgcggtcaccgtctcctcatgagtcgac

SEQ ID NO: 117 - DNA, 42 bp, Artificial; identical to SEQ ID NO: 113
  VLambda-VH Linker

SEQ ID NO: 118 - protein, 14 aa, Artificial
  VLambda-VH Linker
EGKSSGSGSEGAHS

SEQ ID NO: 119 - DNA, 24 bp, Artificial
  Primer VH1 FR3C Upstream
cacagcctacatggagctgagcag

SEQ ID NO: 120 - DNA, 24 bp, Artificial
  Primer VH1 FR3C downstream
ctgctcagctccatgtaggctgtg

SEQ ID NO: 121 - DNA, 24 bp, Artificial
  Primer VH3 FR3Ca upstream
ctgtatctgcaaatgaacagcctg

SEQ ID NO: 122 - DNA, 24 bp, Artificial
  Primer VH3 FR3Ca downstream
caggctgttcatttgcagatacag

SEQ ID NO: 123 - DNA, 24 bp, Artificial
  Primer VH3 FR3Cb upstream
ctgtatctgcaaatgaacagtctg

SEQ ID NO: 124 - DNA, 24 bp, Artificial
  Primer VH3 FR3Cb downstream
cagactgttcatttgcagatacag

SEQ ID NO: 125 - DNA, 24 bp, Artificial
  Primer VH3 FR3Cc upstream
ctgtatcttcaaatgaacagcctg

SEQ ID NO: 126 - DNA, 24 bp, Artificial
  Primer VH3 FR3Cc downstream
caggctgttcatttgaagatacag

SEQ ID NO: 127 - DNA, 27 bp, Artificial
  Primer VH4 FR3C upstream
cagttctccctgaagctgagctctgtg

SEQ ID NO: 128 - DNA, 27 bp, Artificial
  Primer VH4 FR3C downstream
cacagagctcagcttcagggagaactg

SEQ ID NO: 129 - DNA, 75 bp, pEL/tk
  CDS (52)..(75)
ggccaaaaattgaaattttattttttttttttggaatataaagcggccgccatgggcccggccgccaacggcgga

(19) United States
(12) Patent Application Publication
(10) Pub. No.: US 2005/0266425 A1
(43) Pub. Date: Dec. 1, 2005

(54) Methods for producing and identifying multispecific antibodies

(75) Inventors: Maurice Zauderer, Pittsford, NY (US); Mark Paris, West Henrietta, NY (US)

Correspondence Address: STERNE, KESSLER, GOLDSTEIN & FOX PLLC, 1100 NEW YORK AVENUE, N.W., WASHINGTON, DC, 20005, US

(73) Assignee: Vaccinex, Inc., Rochester, NY (US)

(21) Appl. No.: 11/024,251

(22) Filed: Dec. 29, 2004

Related U.S. Application Data
(60) Provisional application No. 60/533,241, filed on Dec. 31, 2003.

(51) Int. Cl. C12Q 1/68; G01N 33/53; C12P 21/06; C12N 5/06

(52) U.S. Cl. 435/6; 435/7.1; 435/455; 435/326

(57) ABSTRACT

The present invention relates to a high efficiency method of expressing multispecific antibodies in eukaryotic cells. The invention is further drawn to a method of producing immunoglobulin heavy and light chain libraries, particularly using the trimolecular recombination method, for expression in eukaryotic cells. The invention further provides methods of selecting and screening for multispecific antibodies, and antigen-binding fragments thereof. The invention also provides kits for producing, screening and selecting multispecific antibodies. Finally, the invention provides multispecific antibodies, and antigen-binding fragments thereof, produced by the methods provided herein.

### CROSS-REFERENCE TO RELATED APPLICATION

The present invention claims the benefit of U.S. Provisional Application No. 60/533,241, filed Dec. 31, 2003, the disclosure of which is incorporated herein by reference in its entirety.

### REFERENCE TO SEQUENCE LISTING APPENDIX

This application includes a “Sequence Listing,” which is provided as an electronic document on a compact disk (CD-R). This compact disk contains the file “Sequence Listing.txt” (92,000 bytes, created on Dec. 28, 2004), which is hereby incorporated by reference in its entirety.

### BACKGROUND OF THE INVENTION

1. Field of the Invention

The present invention relates to a high efficiency method of expressing libraries of multispecific antibodies in eukaryotic cells, a method of producing a plurality of immunoglobulin heavy and light chains for expression of multispecific antibodies in eukaryotic cells, methods of identifying and isolating multispecific immunoglobulins which bind specific antigens or have a desired functional effect, and multispecific immunoglobulins produced by any of these methods.

2. Related Art

Multispecific Antibodies

Monoclonal antibodies offer several important advantages as therapeutics including specificity, effectiveness, generally low toxicity and unlimited reproducibility. Monospecific antibodies recognize a single epitope and can be selected to either activate or repress the activity of a target molecule through this single epitope. Many physiological responses, however, require crosslinking of two or more different proteins or protein subunits to be triggered. An important example is the activation of heteromeric, cell-surface receptor complexes. For these receptor complexes, activation is normally achieved through ligand interaction with multiple domains on different proteins resulting in proximity-associated activation of one or both receptor components. Multispecific antibodies can serve as an alternative means of crosslinking such receptor components. This is advantageous even in situations where a natural ligand exists because of the stability, ease of manufacture and relatively long-half-life of antibodies and because antibodies are not susceptible to inhibitory mechanisms that might limit the activity of natural ligands. Importantly, there may also be previously unidentified pairings of membrane components for which no natural ligand exists but which, because of their associated enzymatic or other physiological activities, would trigger a desired physiological response if they were crosslinked through multispecific antibodies.

Heteromeric receptor complexes represent an important class of activation targets that are associated with virtually every physiologically important signaling pathway. Examples of receptors that could be the target of therapeutic multispecific antibodies include the heterodimeric receptors for Bone Morphogenic Proteins (BMP's) (see, e.g., Groeneveld, E H J and Burger, E H Eur J Endocrinol 142:9-21 (2000)), the heterodimeric receptor complex for Leukemia Inhibitory Factor (LIF) comprised of the two membrane proteins LIFRα and gp130 (see, e.g., (Gearing, D P, et al., EMBO J. 10:2839-48 (1991)), and the heterodimeric receptor for GDNF (glial cell line-derived neruotrophic factor), comprised of the GDNF family receptor a (GFRα1) and the Ret receptor tyrosine kinase (RTK) (see, e.g., Jing, S., et al. Cell 85:1113-24 (1996)). These receptor complexes, as well as the use of multispecific antibodies to activate them, are described in more detail herein.

Multispecific antibodies are being employed in an increasing number of diverse therapeutic applications. Multispecific antibodies are being used either alone or in combination with other chemotherapeutics in cancer imaging and therapy (Tretter et al., J. Chemother. 15:472-479 (2003); Xie et al., Biochem. Biophys. Res. Commun. 14:307-312 (2003); Rossi et al., Clin. Cancer Res. 9:3886S-96S (2003); Dorvillius et al., Tumour Biol. 23:337-347 (2002)); for the treament of infectious diseases (Lindorfer et al., J. Immunol. 167:2240-9 (2001); Bruhl et al., J. Immunol. 166:2420-6 (2001)); and for treatment of autoimmune diseases (Lindorfer et al., J. Immunol. Methos 248:149-66 (2001).

One approach to identify antibodies in a library expression system is to screen recombinant human antibody fragments displayed on bacteriophage (McGunness, et al., Nat. Biotechnol. 14:1149-1154 (1996); Barbas, C. F., III Nat. Med. 1:837-839 (1995); Kay, B. K., et al. (eds.) “Phage Display of Peptides and Proteins” Academic Press (1996)). In phage display methods, functional immunoglobulin domains are displayed on the surface of a phage particle which carries polynucleotide sequences encoding them. In typical phage display methods, immunoglobulin fragments, e.g., Fab, Fv or disulfide stabilized Fv immunoglobulin domains are displayed as fusion proteins, i.e., fused to a phage surface protein. Examples of phage display methods that can be used to make the antibodies include those disclosed in Brinkman U. et al. (1995) J. Immunol. Methods 182:41-50; Ames, R. S. et al. (1995) J. Immunol. Methods 184:177-186; Kettleborough, C. A. et al. (1994) Eur. J. Immunol. 24:952-958; Persic, L. et al. (1997) Gene 187:9-18; Burton, D. R. et al. (1994) Advances in Immunology 57:191-280; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

Since phage display methods normally only result in the expression of an antigen-binding fragment of an immunoglobulin molecule, after phage selection, the immunoglobulin coding regions from the phage must be isolated and re-cloned to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)2 fragments can be employed using methods known in the art such as those disclosed in WO 92/22324; Mullinax, R. L. et al., BioTechniques 12(6):864-869 (1992); and Sawai, H. et al., AJR134:26-34 (1995); and Better, M. et al., Science 240:1041-1043 (1988) (said references incorporated by reference in their entireties).

Immunoglobulin libraries constructed in bacteriophage may derive from antibody producing cells of naïve or specifically immunized individuals and could, in principle, include new and diverse pairings of human immunoglobulin heavy and light chains. Although this strategy does not suffer from an intrinsic repertoire limitation, it requires that complementarity determining regions (CDRs) of the expressed immunoglobulin fragment be synthesized and fold properly in bacterial cells. Many antigen binding regions, however, are difficult to assemble correctly as a fusion protein in bacterial cells. In addition, the protein will not undergo normal eukaryotic post-translational modifications. As a result, this method imposes a selective filter on the antibody specificities that can be obtained.

In principle, it might be possible to identify desired multispecific antibodies utilizing phage display, for example, by expressing 2 different scFv or Fab in a single phage particle and allowing the particle to crosslink mammalian cell surface components. A major technical problem with this approach, however, is that non-specific interactions between phage particles and the mammalian cell surface can result in significant background binding.

There is a need, therefore, for an alternative method to identify mutlispecific immunoglobulin molecules, and multispecific fragments thereof, from an immunoglobulin repertoire that can be synthesized, properly glycosylated and correctly assembled in eukaryotic cells.

Eukaryotic Expression Libraries. A basic tool in the field of molecular biology is the conversion of poly(A)⁺ mRNA to double-stranded (ds) cDNA, which then can be inserted into a cloning vector and expressed in an appropriate host cell. A method common to many cDNA cloning strategies involves the construction of a “cDNA library” which is a collection of cDNA clones derived from the poly(A)⁺ mRNA derived from a cell of the organism of interest. For example, in order to isolate cDNAs which express immunoglobulin genes, a cDNA library might be prepared from pre B cells, B cells, or plasma cells. Methods of constructing cDNA libraries in different expression vectors, including filamentous bacteriophage, bacteriophage lambda, cosmids, and plasmid vectors, are known. Some commonly used methods are described, for example, in Sambrook et al., Molecular Cloning: A Laboratory Manual, 2d Edition, Cold Spring Harbor Laboratory, publisher, Cold Spring Harbor, N.Y. (1990).

Many different methods of isolating target genes from cDNA libraries have been utilized, with varying success. These include, for example, the use of nucleic acid hybridization probes, which are labeled nucleic acid fragments having sequences complementary to the DNA sequence of the target gene. When this method is applied to cDNA clones in transformed bacterial hosts, colonies or plaques hybridizing strongly to the probe are likely to contain the target DNA sequences. Hybridization methods, however, do not require, and do not measure, whether a particular cDNA clone is expressed. Alternative screening methods rely on protein expression in the bacterial host, for example, colonies or plaques can be screened by immunoassay for binding to antibodies raised against the protein of interest. Assays for expression in bacterial hosts are often impeded, however, because the protein may not be sufficiently expressed in bacterial hosts, it may be expressed in the wrong conformation, and it may not be processed, and/or transported as it would be in a eukaryotic system. Many of these problems have been encountered in attempts to produce immunoglobulin molecules in bacterial hosts, as alluded to above.

Accordingly, use of eukaryotic expression libraries to isolate cDNAs encoding immunoglobulin molecules would offer several advantages over bacterial libraries. For example, immunoglobulin molecules, and subunits thereof, expressed in eukaryotic hosts should be functional and should undergo normal posttranslational modification. A protein ordinarily transported through the intracellular membrane system to the cell surface should undergo the complete transport process. Further, use of a eukaryotic system would make it possible to isolate polynucleotides based on functional expression of a protein product. For example, multispecific antibodies could be isolated based on their specificity for given antigens and their effect on antigen-bearing target cells.

With some exceptions, such as cloning of lymphokine cDNAs by expression in COS cells (Wong, G. G., et al., Science 228:810-815 (1985); Lee, F. et al., Proc. Natl. Acad. Sci. USA 83:2061-2065 (1986); Yokota, T., et al., Proc. Natl. Acad. Sci. USA 83:5894-5898 (1986); Yang, Y., et al., Cell 47:3-10 (1986)), many more cDNAs have been isolated from bacterial expression systems than from mammalian expression libraries. There appear to be two principal reasons for this: First, the existing technology (Okayama, H. et al., Mol. Cell. Biol. 2:161-170 (1982)) for construction of large plasmid libraries is difficult to master, and library size rarely approaches that accessible by phage cloning techniques. (Huynh, T. et al., In: DNA Cloning Vol, I, A Practical Approach, Glover, D. M. (ed.), IRL Press, Oxford (1985), pp. 49-78). Second, the existing vectors are, with some exceptions (Wong, G. G., et al., Science 228:810-815 (1985)), often poorly adapted for high level expression. Thus, expression in mammalian hosts previously has been most frequently employed solely as a means of verifying the identity of the protein encoded by a gene isolated by more traditional cloning methods.

More recently, however, the successful use of highly complex poxvirus-based expression libraries, in particular, libraries expressing full-size, fully human immunoglobulin molecules, have been described. The present inventor has demonstrated the identification and isolation of antibodies which bind to a particular desired antigen through use of this unique system. See, e.g., Zauderer, WO 00/028016, published May 18, 2000, and Zauderer, et. al., U.S. Patent Publication US-2002-0123057-A1, published Sep. 5, 2002, both of which are incorporated herein by reference in their entireties.

Tri-molecular recombination is a novel, high efficiency, high titer-producing method for producing recombinant poxviruses. Using the tri-molecular recombination method in vaccinia virus, the present inventor has achieved recombination efficiencies of at least 90%, and titers at least 2 orders of magnitude higher, than those obtained by direct ligation. According to the tri-molecular recombination method, a poxvirus genome is cleaved in a non-essential region to produce two nonhomologous fragments or “arms.” A transfer vector is produced which carries the heterologous insert DNA flanked by regions of homology with the two poxvirus arms. The arms and the transfer vector are delivered into a recipient host cell, allowing the three DNA molecules to recombine in vivo. As a result of the recombination, a single poxvirus genome molecule is produced which comprises each of the two poxvirus arms and the insert DNA.

### SUMMARY OF THE INVENTION

In accordance with one aspect of the present invention, there is provided a method of identifying and isolating polynucleotides which encode a multispecific antibodies (or antibody), or a multispecific fragment thereof, from libraries of polynucleotides expressed in eukaryotic cells, where the multispecific antibody binds to two or more antigenic determinants of interest.

Also provided is a method of identifying polynucleotides which encode a monospecific immunoglobulin molecule, or an antigen-binding fragment thereof, from libraries of polynucleotides expressed in eukaryotic cells, wherein said immunoglobulin molecule is cross-linked to a monospecific antibody of known specificity.

Also provided is a method of constructing libraries of polynucleotides encoding immunoglobulin subunit polypeptides in eukaryotic cells using virus vectors, where the libraries are constructed by trimolecular recombination, and where the immunoglobulin subunit polypeptides are engineered such that they readily combine to form a plurality of multispecific antibodies in eukaryotic cells.

Also provided are methods of screening for soluble multispecific antibodies, or multispecific fragments thereof, expressed from eukaryotic host cells expressing libraries of polynucleotides encoding soluble secreted immunoglobulin molecules, through antigen binding or through detection of an antigen- or cell-specific function induced via binding of the multispecific antibodies (or antibody) to a selected target cell.

### BRIEF DESCRIPTION OF THE FIGURES

FIG. 1 Bispecific antibody constructs
A. Bispecific bivalent antibody with a single fixed light chain, a pre-selected, fixed heavy chain and one variable heavy chain.
B. Bispecific bivalent antibody with one fixed heavy chain, one fixed light chain, one variable heavy chain, and one variable light chain.
C. Bispecific tetravalent antibody with a single fixed light chain, a pre-selected, fixed heavy chain and one variable heavy chain.
D. Bispecific tetravalent antibody with a single fixed light chain, a pre-selected, fixed heavy chain, a second randomized heavy chain which can associate with either the fixed light chain or a randomized light chain in another arm of the antibody.

FIGS. 2A and 2B Construction of pVLE-H5 and pVKE-H5

FIG. 3 Diagram of SF3R1

FIG. 4 Construction of pVHE H5 MBMu

FIG. 5 Construction of pVHE H5 GS

FIGS. 6A and 6B Construction of pVHE H5 MBG1

FIG. 7 Schematic of the Tri-Molecular Recombination Method.

FIG. 8. Nucleotide Sequence of p7.5/tk and pEL/tk promoters. The nucleotide sequence of the promoter and beginning of the thymidine kinase gene for v7.5/tk (SEQ ID NO: 1) and vEL/tk is shown (SEQ ID NO: 129), and the corresponding amino acid sequence including the initiator codon and a portion of the open reading frame for each, designated herein as SEQ ID NO: 2.

FIG. 9 Construction of scFv expression vectors.

FIG. 10 Induction of osteoblast differentiation by BMP-2.

### DETAILED DESCRIPTION OF THE PREFERRED EMBODIMENTS

The present invention is broadly directed to methods of producing and identifying functional, multispecific antibodies, or multispecific fragments thereof, in a eukaryotic system. In addition, the invention is directed to methods of identifying polynucleotides which encode a multispecific antibodies (or antibody), or a multispecific fragment thereof, from complex expression libraries of polynucleotides encoding such immunoglobulin molecules or fragments, where the libraries are constructed and screened in eukaryotic host cells. Further embodiments include isolated multispecific antibodies (or antibody), or multispecific fragment thereof, produced by any of the above methods, and a kit allowing production of such isolated immunoglobulins.

A particularly preferred aspect of the present invention is the construction of complex immunoglobulin libraries in eukaryotic host cells using poxvirus vectors constructed by trimolecular recombination. The ability to construct complex cDNA libraries in a pox virus based vector and to select and/or screen for specific recombinants on the basis of antigen-specific binding or antigen induced signaling in a target cell can be the basis for identification of multispecific immunoglobulins with a variety of well-defined specificities and functions in eukaryotic cells.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

The term “eukaryote” or “eukaryotic organism” is intended to encompass all organisms in the animal, plant, and protist kingdoms, including protozoa, fungi, yeasts, green algae, single celled plants, multi celled plants, and all animals, both vertebrates and invertebrates. The term does not encompass bacteria or viruses. A “eukaryotic cell” is intended to encompass a singular “eukaryotic cell” as well as plural “eukaryotic cells,” and comprises cells derived from a eukaryote.

The term “vertebrate” is intended to encompass a singular “vertebrate” as well as plural “vertebrates,” and comprises mammals and birds, as well as fish, reptiles, and amphibians.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject.

The terms “tissue culture” or “cell culture” or “culture” or “culturing” refer to the maintenance or growth of plant or animal tissue or cells in vitro under conditions that allow preservation of cell architecture, preservation of cell function, further differentiation, or all three. “Primary tissue cells” are those taken directly from tissue, i.e., a population of cells of the same kind performing the same function in an organism. Treating such tissue cells with the proteolytic enzyme trypsin, for example, dissociates them into individual primary tissue cells that grow or maintain cell architecture when seeded onto culture plates. Cell cultures arising from multiplication of primary cells in tissue culture are called “secondary cell cultures.” Most secondary cells divide a finite number of times and then die. A few secondary cells, however, may pass through this “crisis period,” after which they are able to multiply indefinitely to form a continuous “cell line.” The liquid medium in which cells are cultured is referred to herein as “culture medium” or “culture media.” Culture medium into which desired molecules, e.g., immunoglobulin molecules, have been secreted during culture of the cells therein is referred to herein as “conditioned medium.”

The term “polynucleotide” refers to any one or more nucleic acid segments, or nucleic acid molecules, e.g., DNA or RNA fragments, present in a nucleic acid or construct. A “polynucleotide encoding an immunoglobulin subunit polypeptide” refers to a polynucleotide which comprises the coding region for such a polypeptide. In addition, a polynucleotide may encode a regulatory element such as a promoter or a transcription terminator, or may encode a specific element of a polypeptide or protein, such as a secretory signal peptide or a functional domain.

As used herein, the term “identify” refers to methods in which desired molecules, e.g., polynucleotides encoding immunoglobulin molecules with a desired specificity or function, are differentiated from a plurality or library of such molecules. Identification methods include “selection” and “screening.” As used herein, “selection” methods are those in which the desired molecules may be directly separated from the library. For example, in one selection method described herein, host cells comprising the desired polynucleotides are directly separated from the host cells comprising the remainder of the library by binding to an antigen. As used herein, “screening” methods are those in which pools comprising the desired molecules are subjected to an assay in which the desired molecule can be detected. Aliquots of the pools in which the molecule is detected are then divided into successively smaller pools which are likewise assayed, until a pool which is highly enriched for the desired molecule is achieved. For example, in one screening method described herein, pools of antibodies secreted by host cells comprising library polynucleotides encoding immunoglobulin molecules are tested for antigen binding in an ELISA assay or assayed for a defined functional effect on a target cell population.

Immunoglobulins. As used herein, an “immunoglobulin molecule” is defined as a complete, molecular immunoglobulin. Immunoglobulin molecules are also referred to as “antibodies,” and the terms are used interchangeably herein. An “isolated immunoglobulin” refers to an immunoglobulin molecule, or two or more immunoglobulin molecules, which are substantially removed from the milieu of proteins and other substances, and which bind a specific antigen. The term “isolated” is not meant to specify any level of purification.

In certain embodiments, an immunoglobulin molecule comprises four “subunit polypeptides,” i.e., two heavy chains and two light chains (H₂L₂). Thus, by an “immunoglobulin subunit polypeptide” is meant a single heavy chain polypeptide or a single light chain polypeptide. The heavy chain, which determines the “class” of the immunoglobulin molecule, is the larger of the two subunit polypeptides, and comprises a variable region and a constant region. By “heavy chain” is meant either a full-length secreted heavy chain form, i.e., one that is released from the cell, or a membrane bound heavy chain form, i.e., comprising a membrane spanning domain and an intracellular domain. The membrane spanning and intracellular domains can be the naturally-occurring domains associated with a certain heavy chain, i.e., the domain found on memory B-cells, or it may be a heterologous membrane spanning and intracellular domain, e.g., from a different immunoglobulin class or from a heterologous polypeptide, i.e., a non-immunoglobulin polypeptide. As will become apparent, certain aspects of the present invention are preferably carried out using cell membrane-bound immunoglobulin molecules, while other aspects are preferably carried out using secreted immunoglobulin molecules, i.e., those lacking the membrane spanning and intracellular domains. Immunoglobulin “classes” refer to the broad groups of immunoglobulins which serve different functions in the host. For example, human immunoglobulins are divided into five classes, i.e., IgG, comprising a γ heavy chain, IgM, comprising a μ heavy chain, IgA, comprising an α heavy chain, IgE, comprising an ε heavy chain, and IgD, comprising a δ heavy chain. Certain classes of immunoglobulins are also further divided into “subclasses.” For example, in humans, there are four different IgG subclasses, IgG1, IgG2, IgG3, and IgG4 comprising γ-1, γ-2, γ-3, and γ-4 heavy chains, respectively, and two different IgA subclasses, IgA-1 and IgA-2, comprising α-1 and α-2 heavy chains, respectively. It is to be noted that the class and subclass designations of immunoglobulins vary between animal species, and certain animal species may comprise additional classes of immunoglobulins. For example, birds also produce IgY, which is found in egg yolk.

By “light chain” is meant the smaller immunoglobulin subunit which associates with the amino terminal region of a heavy chain. As with a heavy chain, a light chain comprises a variable region and a constant region. There are two different kinds of light chains, kappa and lambda, and a pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

Immunoglobulin subunit polypeptides each comprise a constant region and a variable region. In most species, the heavy chain variable region, or VH domain, and the light chain variable region, or VL domain, combine to form an antigen binding domain comprised of “complementarity determining regions” or CDRs, the portion of an immunoglobulin molecule which specifically contributes to the antigen-binding site for a particular epitope. Generally, heavy and light chains each have three CDRs, which combine to form the antigen binding site of the immunoglobulin. In camelid species, however, the heavy chain variable region, referred to as V_{H}H, forms the entire antigen binding site. Immunoglobulins that possess the classic H₂L₂ structure contain two such antigen binding sites. The main differences between camelid V_{H}H variable regions and those derived from conventional antibodies (VH) include (a) more hydrophobic amino acids in the light chain contact surface of VH as compared to the corresponding region in V_{H}H, (b) a longer CDR3 in V_{H}H, and (c) the frequent occurrence of a disulfide bond between CDR1 and CDR3 in V_{H}H. A large repertoire of variable regions associated with heavy and light chain constant regions are produced upon differentiation of antibody-producing cells in an animal through rearrangements of a series of germ line DNA segments which results in the formation of a gene which encodes a given variable region. Further variations of heavy and light chain variable regions take place through somatic mutations in differentiated cells. The structure and in vivo formation of immunoglobulin molecules is well understood by those of ordinary skill in the art of immunology. Concise reviews of the generation of immunoglobulin diversity may be found, e.g., in Harlow and Lane, Antibodies, A Laboratory Manual Cold Spring Harbor Laboratory, Cold Spring Harbor, N.Y. (1988) (hereinafter, “Harlow”); and Roitt, et al., Immunology Gower Medical Publishing, Ltd., London (1985) (hereinafter, “Roitt”). Harlow and Roitt are incorporated herein by reference in their entireties.

An “antigen binding domain” of an immunoglobulin molecule generally, but not invariably, consists of at least a portion of the variable domain of one heavy chain and at least a portion of the variable domain of one light chain, held together by disulfide bonds. Thus, an immunoglobulin having four subunit polypeptides in the H₂L₂ configuration has two antigen binding domains, and is therefore referred to herein as a “bivalent” immunoglobulin, or a “bivalent” antibody. Similarly, an immunoglobulin molecule which has three antigen binding domains, e.g., H₃L₃, would be referred to herein as being “trivalent,” and an immunoglobulin molecule which has four antigen binding domains, e.g., H₄L₄, would be referred to as being “tetravalent.” An immunoglobulin molecule of the present invention may have a larger valency as well, for example a 5-fold, 6-fold, 7-fold, 8-fold, 9-fold or 10-fold valency.

In a naturally occurring bivalent immunoglobulin molecule, the two antigen binding domains are identical, i.e., they have the same amino acid sequence, and they bind the same antigenic epitope (i.e., they have the same “specificity”). Such an immunoglobulin is referred to herein as being “monospecific.” Conversely, where one or more antigen binding domains of an immunoglobulin molecule are different than one or more other antigen binding domains of the same immunoglobulin molecule, i.e., the antigen binding domains have different amino acid sequences and bind to different epitopes, they are referred to herein as being “multispecific” (e.g., a multispecific antibody). In one embodiment, a multispecific antibody of the present invention comprises antigen binding domains with two different specificities, and is referred to herein as a “bispecific antibody.” A multispecific antibody may have any number of antigen binding domains, and the number of antigen binding domains need not be equal to the number of specificities, as long as the number of valencies is greater than or equal to the number of specificities. Thus a bispecific antibody might be bivalent, trivalent, tetravalent, or have even a higher valency.

In some instances, e.g., immunoglobulin molecules derived from camelid species or engineered based on camelid immunglobulins, a complete immunoglobulin molecule may consist of heavy chains only, with no light chains. See, e.g., Hamers-Casterman et al., Nature 363:446-448 (1993). Such an immunoglobulin may be multivalent and/or multispecific as described above, except that a single antigen binding domain consists of just a heavy chain variable region.

Immunoglobulins further have several effector functions mediated by binding of effector molecules. For example, binding of the C1 component of complement to an immunoglobulin activates the complement system. Activation of complement is important in the opsonization and lysis of cell pathogens. The activation of complement also stimulates the inflammatory response and may also be involved in autoimmune hypersensitivity. Further, immunoglobulins bind to cells via the Fc region, with an Fc receptor binding site on the antibody Fc region binding to an Fc receptor (FcR) on a cell. There are a number of Fc receptors which are specific for different classes of antibody, including, but not limited to, IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Immunoglobulins of the present invention may be from any animal origin including birds, fish, and mammals. Preferably, the antibodies are of human, mouse, dog, cat, rabbit, goat, guinea pig, camel, llama, horse, or chicken origin. In a preferred aspect of the present invention, immunoglobulins are identified which specifically interact with antigens of the same species origin, e.g., human immunoglobulins which specifically bind human antigens.

The immunoglobulins of the present invention are “multispecific”, meaning that they recognize and bind to two or more different epitopes present on one or more different antigens (e.g., proteins) at the same time. Multispecific immunoglobulins of the present invention include antibodies which are bispecific and monovalent for each specificity (termed “bispecific bivalent”) and antibodies which are bispecific and bivalent for each specificity (termed “bispecific tetravalent antibodies”). Bispecific bivalent antibodies, and methods of making them, are described, for instance in U.S. Pat. Nos. 5,731,168; 5,807,706; 5,821,33; and U.S. Appl. Publ. Nos. 2003/020734 and 2002/0155537, the disclosures of all of which are incoporated by reference herein. Bispecific tetravalent antibodies, and methods of making them are described, for instance, in WO 02/096948 and WO 00/44788, the disclosures of both of which are incorporated by reference herein. By combining the methods described in these publications, monospecific and bispecific antibodies could be combined into tetravalent antibodies with one, two, three or four different specificities distributed among a total of four antigen binding domains.

In another embodiment, the present invention is drawn to methods to produce and identify, i.e., select or alternatively screen for, polynucleotides which singly or collectively encode a monospecific immunoglobulin molecule, e.g., a monospecific bivalent antibody, or an antigen-binding portion thereof, where the immunoglobulin molecule is crosslinked or covalently bound to another immunoglobulin molecule with a different specificity, to form a bispecific antibodies (or antibody), e.g., a bispecific, tetravalent antibody, after secretion or extraction from the producing cell. According to this embodiment, the polynucleotides are identified through binding of the bispecific antibody to at least two different epitopes, thereby linking those epitopes. Linkage of the epitopes then elicits a detectable signal allowing identification of the bispecific antibody of interest. Bispecific bivalent antibodies of the invention comprise two heavy and two light chains (H₂L₂), forming two different antigen binding domains. Each bispecific bivalent antibody may comprise two non-identical light and two non-identical heavy chains, or antigen-binding portions thereof; they may comprise two non-identical heavy chains and two identical light chains, or antigen-binding portions thereof; or they may comprise two non-identical light chains and two identical heavy chains, or antigen-binding portions thereof (FIGS. 1A and 1B). The heavy and light chains of bispecific bivalent antibodies combine to form two non-identical antigen binding domains, each with different specificity. The two non-identical antigen binding domains of bispecific bivalent antibodies may differ by as little as one amino acid.

Multispecific tetravalent antibodies of the invention are typically comprised of a total of four heavy and four light chains (H₄L₄), or antigen-binding portions thereof. Multispecific tetravalent antibodies of the invention comprise four total antigen binding domains, at least one of which is different than the other three. For example, a multispecific tetravalent antibody of the invention may comprise three antigen binding domains which are identical to each other, and one non-identical antigen binding domain, or two antigen binding domains which are identical to each other, and two other antigen binding domains which are also identical to each other but which are different than the first two antigen binding domains (bispecific tetravalent antibodies); two antigen binding domains which are identical to each other, and two antigen binding domains which are different from all other antigen binding domains in the molecule (a trispecific tetravalent antibody); or four non-identical antigen binding domains (a tetraspecific tetravalent antibody).

In certain embodiments, a multispecific tetravalent antibody of the invention is bispecific, and comprises two monospecific bivalent immunoglobulin molecules or antigen binding fragments thereof, each monospecific bivalent immunoglobulin molecule binding to a different epitope. The two monospecific bivalent immunoglobulin molecules, or fragments thereof may be attached to each other in various ways, for example they may be covalently or non-covalently bound or they may be cross linked by a third immunoglobulin molecule which recognizes constant region domains of the first two immunoglobulin molecules. Methods to attach two monovalent bispecific antibodies together to form a bispecific tetravalent antibody of the present invention are described in more detail below.

In other embodiments, the multispecific tetravalent antibody is trispecific, comprising one monospecific bivalent immunoglobulin molecule attached to one bispecific bivalent immunoglobulin molecule, the bispecific bivalent immunoglobulin having two non-identical antigen binding domains which bind to two different epitopes, and the monospecific bivalent immunoglobulin having two identical antigen binding domains which bind to the same epitope, but different from the epitopes bound by the bispecific bivalent immunoglobulin. In still other embodiments, the multispecific tetravalent antibody is tetraspecific, comprising two monovalent bispecific bivalent antibodies attached to each other, each antibody having two non-identical antigen binding domains which bind to two different epitopes, for a total of four different specificities.

As used herein, an “antigen-binding fragment” of an immunoglobulin molecule is any fragment or variant of an immunoglobulin molecule which retains at least one antigen binding domain. Antigen-binding fragments include, but are not limited to, Fab, Fab′ and F(ab′)₂, Fd, single-chain Fvs (scFv), single-chain immunoglobulins (e.g., wherein a heavy chain, or portion thereof, and light chain, or portion thereof, are fused), disulfide-linked Fvs (sdFv), diabodies, triabodies, tetrabodies, scFv minibodies, Fab minibodies, and dimeric scFv and any other fragments comprising a VL and a VH domain in a conformation such that a specific CDR is formed. Antigen-binding fragments may also comprise a V_{H}H domain derived from a camelid antibody. The V_{H}H may be engineered to include CDRs from other species, for example, from human antibodies. Alternatively, a human-derived heavy chain VH fragment may be engineered to resemble a single-chain camelid CDR, a process referred to as “camelization.” See, e.g., Davies J., and Riechmann, L., FEBS Letters 339:285-290 (1994), and Riechmann, L., and Muyldermans, S., J. Immunol. Meth. 231:25-38 (1999), both of which are incorporated herein by reference in their entireties. Of course, for an “antigen binding fragment” to be multispecific, e.g., bispecific, such a fragment must retain at least two antigen binding domains, that is it must be at least bivalent, e.g., as in an F(ab′)₂ fragment, diabodies, triabodies, tetrabodies, scFv minibodies, Fab minibodies, or dimeric scFv. Alternatively, one or more “antigen binding fragments” with distinct sequences and specificities, each either a monovalent or a bivalent fragment, can be linked to each other or to an intact antibody to create a multispecific antigen binding complex. Some methods for non-covalently or covalently linking two different antibodies or antibody fragments are described below. As will be familiar to those well-practiced in the art, these and other methods can also be adapted to create the multispecific antigen binding complexes described above.

Antigen-binding immunoglobulin fragments, including single-chain immunoglobulins, may comprise the variable region(s) alone or in combination with all or part of the following: a heavy chain constant domain, or portion thereof, e.g., a CH1, CH2, CH3, transmembrane, and/or cytoplasmic domain, linked to the carboxyl terminus of the heavy chain variable region, and a light chain constant domain, e.g., a C-kappa or C-lambda domain, or portion thereof linked to the carboxyl terminus of the light chain variable region. Also included in the invention are any combinations of variable region(s) and CH1, CH2, CH3, C-kappa, C-lambda, transmembrane and cytoplasmic domains. In certain embodiments, especially in the case of tetravalent antibodies, the Ig fragments lack the CH2 domain, or a portion thereof.

As is known in the art, Fv comprises a VH domain and a VL domain, Fab comprises VH joined to CH1 and paired with a light chain, an Fab minibody comprises a fusion of CH3 domain to Fab, etc. As is known in the art, scFv comprises VH joined to VL by a peptide linker, usually 15-20 residues in length, diabodies comprise scFv with a peptide linker about 5 residues in length, triabodies comprise scFv with no peptide linker, tetrabodies comprise scFv with peptide linker 1 residue in length, a scFv minibody comprises a fusion of CH3 domain to scFv, and dimeric scFv comprise a fusion of two scFvs in tandem using another peptide linker (reviewed in Chames and Baty, FEMS Microbiol. Letts. 189:1-8 (2000)). Preferably, an antigen-binding immunoglobulin fragment includes both antigen binding domains, i.e., VH and VL. Other immunoglobulin fragments are well known in the art and disclosed in well-known reference materials such as those described herein.

In certain embodiments, the present invention is drawn to methods to identify, i.e., select or alternatively screen for, polynucleotides which singly or collectively encode multispecific antibodies, multispecific fragments thereof, or multispecific antibodies or fragments thereof with specific antigen-related functions. In related embodiments, the present invention is drawn to isolated multispecific antibodies encoded by the polynucleotides identified by these methods.

In certain embodiments, multispecific antibodies are bispecific. In one embodiment of the invention, bispecific antibodies comprise one fixed, pre-determined antigen specificity expressed in association with a second variable specificity which is produced and identified according to the methods disclosed herein. Different combinations of the fixed and variable specificities may be screened for a desired functional effect upon crosslinking the two specific epitopes on the surface membrane of a target cell.

In another embodiment of the invention, bispecific antibodies comprise two variable specificities which are produced and are identified, either sequentially or simultaneously, according to the methods disclosed herein. The complexity of such libraries may be reduced by first screening monospecific heavy and light chain libraries for polynucleotides which encode a heavy chain and a light chain pair which comprise an antigen binding domain which binds to a a surface membrane of interest of a target cell and subsequently isolating these polynucleotides to express as a “fixed” specificity or as a sublibrary of polynucleotides of more limited diversity as described above for identifying polynucleotides encoding additional subunit polypeptides of bispecific antibodies, and as will be further described below.

Multiple methods exist for construction of bispecific antibodies with either one fixed and one variable specificity or two variable specificities comprised of subunit polypeptides encoded by polynucleotide libraries constructed and expressed employing the methods and vectors described herein. Three methods are described here in detail: a) formation of bispecific bivalent antibodies by introduction of complementing “heterodimerization domains” in the immunoglobulin heavy chain constant regions as described below; b) formation of intracellular bispecific tetravalent antibodies by spontaneous association of CH2 domain-deleted monovalent monospecific antibodies; and c) extracellular formation of bispecific tetravalent antibodies by crosslinking, e.g., with a third antibody. Methods to identify such bispecific antibodies are disclosed herein.

Certain methods described herein comprise a multistep identification process. In the first identification step, a polynucleotide encoding an immunoglobulin subunit polypeptide, i.e., either a first heavy chain or a light chain, is identified from a first library of polynucleotides each encoding that subunit polypeptide, by introducing the library into a population of eukaryotic host cells, and expressing the immunoglobulin subunit polypeptides encoded by the first library in combination with one or more other species of immunoglobulin subunit polypeptides, where the latter immunoglobulin subunit polypeptides are not the same type as the immunoglobulin subunit polypeptides encoded by the first library, i.e., if the immunoglobulin subunit polypeptides encoded by the first library are first heavy chain polypeptides, the additional immunoglobulin subunit polypeptides will be light chain polypeptides and optionally, second heavy chain polypeptides. The distinctions between a “first” heavy chain polypeptide and a “second” heavy chain polypeptide may include, but are not limited to, different specifities or complementary heterodimerization domains, described in more detail herein.

At the same time or following identification of one or more polynucleotides from the first library encoding one or more immunoglobulin subunit polypeptides, one or more additional immunoglobulin subunit polypeptides as listed above may be identified that pair with the immunoglobulin subunit polypeptide(s) encoded by the first library, to enable bispecific antigen recognition. The identification of first heavy chain-, second heavy chain-, and one or more light chain-encoding polynucleotides may be simultaneous or sequential. Simultaneous selection simply means that first heavy chain-, second heavy chain-, and one or more light chain-encoding polynucleotides of a first, a second, and/or a third polynucleotide libraries are produced in and identified and recovered from the same host cells in the same identification step. In certain embodiments, the first heavy chain-, second heavy chain-, and one or more light chain-encoding polynucleotides are expressed as DNA recombinants in an infectious viral vector. In a most preferred embodiment, the infectious vector is the vaccinia virus vector described below.

If identified sequentially, in the first identification step polynucleotides encoding either first heavy chain or light chain subunit polypeptide(s) encoded by the first library are recovered from host cells that comprise a polynucleotide encoding either: fixed immunoglobulin subunit polypeptides with known specificity, of the type that combine with the subunit polypeptides encoded by the first library, i.e., encoding a second heavy chain subunit polypeptide and either a library encoding a plurality of first heavy chain subunit polypeptide or one or more light chain subunit polypeptides; or polynucleotides of a second library and optionally a third library encoding a plurality of immunoglobulin subunit polypeptides of the type that combine with the subunit polypeptides encoded by the first library, i.e., encoding a plurality of second heavy chain subunit polypeptides and either a plurality of first heavy chain subunit polypeptides or a plurality of light chain subunit polypeptides, to form bispecific antibodies or bispecific fragments thereof comprising at least two heavy/light chain pairs with non-identical antigen binding domains, where the latter polynucleotides or libraries of polynucleotides are in a form that is efficiently expressed, but not readily recovered. Identification and recovery of polynucleotides of the first library is carried out via binding of a bispecific antibody of interest to at least two non-identical epitopes of one or more antigens, e.g., antigens expressed on the surface of a target cell, where the binding elicits a detectable signal, e.g., proliferation, functional activation, differentiation or apoptosis of the target cell. By “functional activation” is meant inducing a physiological response characteristic of that host cell type, e.g. secretion of a specific cytokine. One or more round of enrichment may be carried out, as described in detail below.

In a second identification step, polynucleotides recovered from the first library and any other libraries screened in the first identification step are isolated, and are then put in a form that is efficiently expressed but not readily recovered, and are transferred into and expressed in host cells in which a second and/or third library of polynucleotides encoding the other immunoglobulin subunit polypeptide(s) described above are expressed in a different form that is readily recovered, thereby allowing identification of one or more polynucleotides encoding a second heavy chain subunit polypeptide and either a first heavy chain subunit polypeptide or one or more light chain subunit polypeptides (i.e., the subunit polypeptide not isolated in the first identification step) which, when combined with the immunoglobulin subunit polynucleotide encoded by the earlier-isolated polynucleotide, form a functional bispecific antibodies (or antibody), or bispecific antigen-binding fragment thereof, which recognizes at least two non-identical epitopes of one or more antigens, e.g., antigens expressed on the surface of a target cell, where the binding elicits a detectable signal, e.g., proliferation, functional activation, differentiation, or apoptosis of the target cell. In one embodiment, the form of polynucleotides that can be expressed but not readily recovered from host cells are DNA recombinants in the vaccinia virus vector described below which have been rendered replication deficient by crosslinking DNA of the viral genome through treatment with psoralin and irradiation with UV light. Again, one or more rounds of enrichment may be carried out.

Subsequent identification steps may be performed, identifying additional immunoglobulin subunit polypeptides which when substituted for or combined with one of the initially selected subunit polypeptides further enhance the ability to recognize specific antigens and/or perform a specific function.

In a most preferred embodiment, bispecific antibodies are identified by inducing a detectable physiological effect, e.g., proliferation, functional activation, apoptosis or differentiation of target cells. In every case, it can be determined following isolation of the bispecific antibody whether one or both antigen specificities are required to elicit this physiological effect on the target cells. As will be evident to those of ordinary skill in the art, this can be determined by testing individually the activity of antibodies produced by cells that express each pair of immunoglobulin heavy and light chains isolated from cells producing the bispecific antibodies individually or, if monovalency is thought to be required, by testing antibodies produced by cells that express one pair of the isolated immunoglobulin heavy and light chains together with a second arbitrarily selected heavy chain, light chain or heavy and light chain combination. In another embodiment, it is possible to detect a desired bispecific antibody by direct binding to antigen. For example, to select a bispecific antibody reactive with two different soluble antigens, one antigen could be bound to a substrate and the second antigen labeled with a fluorescent tag. Only bispecific antibodies with specificity for both antigens will bind to the substrate and also bind the antigen with fluorescent tag and evince a fluorescent signal.

In certain embodiments, the immunoglobulin subunit polypeptides are capable of forming bispecific bivalent antibodies, i.e. antibodies with two heavy chains and two light chains which form two non-identical antigen binding domains. Methods of making bispecific antibodies are described, for instance, in U.S. Pat. Nos. 5,731,168; 5,807,706; 5,821,333, and 5,932,448; and U.S. Appl. Publ. Nos. 2003/020734 and 2002/0155537, the disclosures of all of which are incoporated by reference herein.

In certain embodiments, bispecific bivalent antibodies form through a “heterodimerization domain,” which promotes stable interaction of two non-identical immunoglobulin heavy chain polypeptides in the antibodies. As used herein, a “heterodimerization domain” refers to a region in an heavy chain subunit polypeptide which interacts with a region of another different heavy chain subunit polypeptide. In certain embodiments, the heterodimerization domain is located in the constant region of the heavy chain, for example, in the CH3 region of the heavy chain. A heterodimerization domain promotes interaction between a first heavy chain and a different second heavy chain, i.e., promotes the formation of heterodimers of heavy chains, thereby increasing the yield of a desired bispecific bivalent antibody with two non-identical heavy chains. Interaction may be promoted at the heterodimerization domain by the formation or insertion of functional groups including, but not limited to protuberance-into-cavity complementary regions; non-naturally occurring disulfide bonds; leucine zipper; hydrophobic regions; and hydrophilic regions. Other functional groups which could promote interaction at a heterodimerization domain would be readily apparent to one of ordinary skill in the art. One or more of these types of heterodimerization domain functional groups may be present in the same immunoglobulin subunit polypeptide. Functional groups promoting interaction at the heterodimerization domains of a first heavy chain subunit polypeptide and a second heavy chain subunit polypeptide must be complementary, i.e., they must interact with each other. Such complementary heterodimerization domains are conveniently referred to herein as a first heterodimerization domain and a second heterodimerization domain.

“Protuberances” are constructed by replacing small amino acid side chains from the interface of a first immunoglobulin heavy chain, with larger side chains. Residues for the formation of a protuberance include, but are not limited to naturally occurring amino acid residues such as arginine (R), phenylalanine (F), tyrosine (Y) and tryptophan (W). In one embodiment, the original residue for the formation of the protuberance has a small side chain volume, such as alanine, asparagine, aspartic acid, glycine, serine, threonine or valine.

Compensatory “cavities” of identical or similar size to the protuberances are optionally created on the interface of a second immunoglobulin heavy chain by replacing large amino acid side chains with smaller ones (e.g. alanine or threonine). Exemplary residues for the formation of a cavity include, but are not limited to naturally occurring amino acid residues such as alanine (A), serine (S), threonine (T) and valine (V). In one embodiment, the original residue for the formation of the protuberance has a large side chain volume, such as tyrosine, arginine, phenylalanine or tryptophan.

Where a suitably positioned and dimensioned protuberance or cavity exists at the interface of a first immunoglobulin heavy chain, it is only necessary to engineer a corresponding cavity or protuberance on a second immunoglobulin heavy chain, at the adjacent interface. Thus, two separate libraries of polynucleotides encoding immunoglobulin heavy chains are engineered to express the protuberance and the cavity, respectively. Examples of mutations of the CH3 domain for promoting heterodimerization of heavy chains are T366Y/Y407′T; T366W/Y407′A; F405A/T394′W; Y407T/T366′Y; T366Y/F405′A; T394W/Y407′T; T366W:F405W/T394′S:Y407′A; F405W:Y407A/T366′W:T394′S; F405W/T394′S; and T366W/T366′S:L368′A:Y407′V. Mutations are denoted by the wild-type residue followed by the position using the Kabat numbering system (Kabat et al., Sequences of Proteins of Immunological Interest, National Institutes of Health, Bethesda, Md., ed. 5, (1991)) and then the replacement residue in single-letter code. Multiple mutations are denoted by listing component single mutations separated by a colon. Mutations on complementary heavy chains are denoted by a slash, with a prime (′) signifying the complementary chain.

Non-naturally occurring disulfide bonds are constructed by replacing on the first immunoglobulin heavy chain a naturally occurring amino acid with a free thiol-containing residue, such as cysteine, such that the free thiol interacts with another free thiol-containing residue inserted on the second immunoglobulin heavy chain such that a disulfide bond is formed between the first and second immunoglobulin heavy chains. Two separate libraries of polynucleotides encoding immunoglobulin heavy chains are constructed to contain one or more engineered thiol-containing residues which preferentially interact with the free thiol-containing residue on the complementary heavy chain. In certain embodiments, the non-naturally occurring disulfide bonds are located in the CH3 domain of the heavy chain(s). In certain embodiments, the mutations favor formation of immunoglobulin heavy chain heterodimers over homodimers, i.e., mutations which favor the binding of two non-identical immunoglobulin heavy chains which can be isolated individually over two identical immunoglobulin subunit polypeptides or two non-identical subunit polypeptides which cannot be readily isolated individually. Examples of such mutations include K392C/D399′C, S354C/Y349′C, E356/Y349′C, and E357C/Y349′C, denoted as described above.

In further embodiments, the CH3 domains encoded by two separate libraries of heavy chain-encoding polynucleotides are engineered to contain mutations for both a non-naturally occurring thiol-containing residue and a protuberence-into-cavity mutation.

Leucine zippers are specific amino acid sequences about 20 to 40 residues in length, with leucine typically occuring at every seventh residue. Such zipper sequences form amphipathic alpha-helices, with the leucine residues lined up on the hydrophobic side for dimer formation. The present invention includes separate libraries of polynucleotides encoding heavy chain subunit polypeptides with complementary constant region leucine zippers which favor the formation of heavy chain heterodimers, for instance peptides corresponding to the leucine zippers of the Fos and Jun protein. Methods of making bispecific antibodies with leucine zippers are described, for example, in U.S. Pat. No. 5,932,448.

Certain embodiments include the selection of bispecific antibodies with one fixed and one variable specificity. As used herein, a “fixed” immunoglobulin subunit polypeptide denotes a single polypeptide (or one or more copies of that polypeptide) with a known amino acid sequence in the variable region, e.g., in the complementarity determining regions. A “fixed” immunoglobulin subunit polypeptide, e.g., either a heavy chain or a light chain, when combined with a complementary “fixed” immunoglobulin subunit polypeptide, forms an antigen binding domain which binds to a known and well-defined epitope on an antigen, and will bind with a reproducible affinity. Polynucleotides encoding a “fixed” immunoglobulin subunit polypeptide will, in all instances, encode immunoglobulin subunit polypeptides with an identical complementarity determining region. As used herein, a plurality of immunoglobulin subunit polypeptides with a “defined” specificity refers to a group of immunoglobulin subunit polypeptides, e.g., heavy chains or light chains, which generally combine with a complementary immunoglobulin subunit polypeptide to form antigen binding domains with related specificity. For example, a plurality of heavy chain subunit polypeptides with defined specificity might combine with fixed or defined light chains to form antigen binding domains which bind to the same antigen, which bind to the adjacent or overlapping epitopes, or which bind to the same epitope, but with differing affinities. In other words, a group of immunoglobulin subunit polypeptides with “defined” specificity are related based on the antigen or epitope they bind to, and not by their structure or amino acid sequences, which may be related, but need not be related.

In these embodiments a library of polynucleotides encoding diverse first immunoglobulin heavy chains each with a heterodimerization domain in the constant region is introduced into host cells together with (i) a library of polynucleotides encoding diverse immunoglobulin light chains; (ii) a polynucleotide encoding a single fixed heavy chain or polynucleotides encoding a plurality of heavy chains that contribute to a defined antigen specificity when paired with certain light chains, and have been modified to express a complementing heterodimerization domain in the constant region; and (iii) a polynucleotide encoding a single fixed light chain or polynucleotides encoding a plurality of light chains that when paired with the defined heavy chain(s) of (ii) immediately above creates a binding site with the defined specificity. The heterodimerization domain encoded by the library of polynucleotides may be either a “protuberance” or a “cavity.” If a protuberance, the complementing heterodimerization domain carried on the single fixed heavy chain is a cavity. If a cavity, the complementing heterodimerization domain on the single fixed heavy chain is a protuberance. Similarly, if heterodimerization is promoted by a leucine zipper, the heterodimerization domain may be either Fos or Jun; if Fos, the complementing heterodimerization is Jun; if Jun, the complementing heterodimerization domain is Fos.

In a related embodiment, the library of polynucleotides encoding diverse immunoglobulin light chains is omitted in the method of the previous paragraph and the only light chain-encoding polynucleotides encode the defined light chain(s) which when paired with the defined heavy chain(s) create an antigen binding domain with the defined specificity. In a preferred embodiment only a polynucleotide that encodes a single fixed light chain is introduced into host cells together with the polynucleotides that encode diverse immunoglobulin heavy chains and defined heavy chains. This has the effect of forcing selection of bispecific bivalent antibodies with two different antigen specificities, one variable and one pre-defined, that employ the same light chain.

At the same time, prior to, or subsequent to identification of the polynucleotide(s) encoding one or more first immunoglobulin heavy chains, polynucleotides encoding one or more second immunoglobulin light chains may be identified which in combination with the first and/or second heavy chain subunit polypeptides encoded by one or more heavy chain libraries and any fixed immunoglobulin heavy and/or light chains, comprise bispecific antibodies with a selected binding specificity or function. In subsequent steps, polynucleotides encoding additional immunoglobulin subunit polypeptides may be identified which when substituted for one of the initially identified subunit polypeptides further enhance the ability to recognize specific antigenic determinants and/or perform a specific function.

The libraries of polynucleotides encoding heavy and light chain subunit polypeptides may be constructed for expression as either bispecific membrane receptors or secreted bispecific antibody molecules and selected through interaction with antigen or target cells for the desired specificity and function as described elsewhere (US 20020123057A1, published Sep. 5, 2002).

In certain embodiments, the complexity of either or both of the first heavy chain-, and light chain-encoding polynucleotide libraries is reduced by prior identification and isolation of polynucleotides encoding monospecific bivalent antibodies that bind to a surface epitope of a target cell of interest, followed by incorporation of the variable regions of these isolated polynucleotides into sublibraries comprising polynucleotides that encode heavy chain constant regions with heterodimerization domains for selection of bispecific bivalent antibodies.

In another embodiment, bispecific bivalent antibodies are selected with two variable specificities. In this embodiment, two libraries of polynucleotides, each encoding diverse immunoglobulin heavy chains are constructed, one library encoding first heavy chains with a first heterodimerization domain in its constant region and the other library encoding second heavy chains with a complementing second heterodimerization domain in its constant region. These libraries are introduced into host cells together with a single library of polynucleotides encoding diverse immunoglobulin light chains, or alternatively with one or more polynucleotides encoding defined light chains of limited diversity. As noted above, the complexity of one or more of these libraries may be reduced by first identifying, and then isolating polynucleotides encoding monospecific antibodies that bind to a surface epitope of the target cell of interest followed by incorporation of the heavy and light chain variable region-encoding portions of the isolated polynucleotides into one light chain and two heavy chain polynucleotide sublibraries, encoding heavy chain constant regions with complementary first and second heterodimerization domains, respectively, for selection of bispecific bivalent antibodies.

FIG. 1A shows a bispecific bivalent antibody comprised of a single fixed light chain which confers a desired specificity when associated with a pre-selected, fixed heavy chain in one arm of the antibody and which can also associate with a second randomized heavy chain in the other arm of the antibody. In host cells into which polynucleotides are introduced that encode one heavy chain of a library of diverse heavy chains with a heterodimerization domain; along with a polynucleotide encoding a fixed heavy chain with a complementing heterodimerization domain, and a polynucleotide encoding a fixed light chain, 100% of the antibodies are expected, on average, to be a productive combination in the sense of comprising two different antigen combining sites of which one has a pre-determined specificity and one a variable specificity.

FIG. 1B shows a bispecific bivalent antibody comprised of a single fixed light chain which confers a desired specificity when associated with a pre-selected, fixed heavy chain in one arm of the antibody; and a second randomized heavy chain which can associate with either the fixed light chain or a randomized light chain in the other arm of the antibody. In host cells into which polynucleotides are introduced that encode one heavy chain of a library of diverse heavy chains with a heterodimerization domain; along with a polynucleotide encoding a fixed heavy chain with a complementing heterodimerization domain, a polynucleotide encoding a fixed light chain, and polynucleotides encoding one second light chain from a library of diverse light chains, 25% of the antibodies produced in each host cells are expected, on average, to be a productive combination in the sense of having two different antigen combining sites of which one has a pre-determined specificity comprised of the fixed heavy and light chains and one a variable specificity comprised of the randomized heavy chains and and light chains.

In other embodiments, the immunoglobulin subunit polypeptides identified according to the present invention are capable of forming bispecific tetravalent antibodies. Bispecific tetravalent antibodies of the present invention comprise four heavy and four light chains (H₄L₄) for a total of four antigen-binding domains, and may be assembled, for example, from monospecific bivalent antibodies either intracellularly or extracellularly via a “means for tetramerization,” normally associated with the heavy chain constant regions as described and referenced herein. Assembly of bispecific tetravalent antibodies intracellularly is described, for instance, in WO 02/096948, the disclosure of which is incorporated by reference herein in its entirety.

As used herein, a “means for tetramerization” is any added structure or modification which promotes the association of four heavy and light chain pairs, e.g., two monospecific bivalent antibodies, one monospecific bivalent antibody and two univalent heavy and light chain pairs, or four univalent heavy and light chain pairs into a tetrameric antibody. A “heavy and light chain pair” as used herein may be a single chain molecule, such as an ScFv. A means for tetramerization may include the covalent attachment of two or more heavy and light chain pairs, e.g., as fusion proteins, a modification to one or more of the heavy chain constant regions, e.g., a modification to the heavy chain sequence or the addition of a peptide or chemical conjugate to one or more heavy chains, or the use of an independent structure capable of joining two or more heavy and light chain pairs, e.g., an antibody which specifically binds to the constant regions of two or more heavy and light chain pairs.

One example of a “means for tetramerization” is the deletion of the CH2 domains of the heavy chains. In particular, pairs of heavy chains of a bivalent antibody which lack all or a part of the CH2 domain between the hinge and the CH3 domain can spontaneously assemble to form tetravalent antibodies held together through non-covalent interactions.

Other “means for tetramerization” include, but are not limited to the formation of fusion proteins comprising all or part of two different antigen binding domains, covalent attachment of two monospecific bivalent antibodies via engineered disulfide linkages or chemical cross-linking such as, for example, reaction of bis-maleimide with free sufhydryl groups; affinity interactions such as biotin-avidin wherein biotinylated heavy chain constant regions are cross linked by binding to avidin, or coil-coil interactions of an interactive protein domain such as may be derived from the collagen sequence which when synthesized as a fusion protein with the heavy chain constant region generates a pentameric association; or cross linking of two antibodies by a third antibody. Such means for tetramerization are described in more detail below.

In certain embodiments, polynucleotides are identified which encode heavy chains of the immunoglobulin molecules lacking all or part of a CH2 domain. The CH2 domain of a human IgG Fc region usually extends from about residue 231 to residue 340 using conventional numbering schemes (see Kabat, above). Accordingly, a heavy chain according to this embodiment will have at least about one amino acid from about amino acid 231 to about amino acid 340 deleted. For example, a heavy chain according to this embodiment will have at least about 1, at least about 5, and least about 10, at least about 15, and least about 20, at least about 30, at least about 40, at least about 50 at least about 60, at least about 70, at least about 80, at least about 90, or at least about 100 amino acids from about amino acid 231 to about amino acid 340 deleted. Alternatively, the entire region from about amino acid 231 to about amino acid 340 is deleted. Those of ordinary skill in the art will appreciate that the amino acid coordinates of the CH2 domain of an immunoglobulin heavy chain will vary depending on the heavy chain isotype and also depending on the number of amino acids in the variable region. The skilled artisan can easily identify the CH2 domain in any given heavy chain subunit polypeptide, and can delete all or part of it according to the general guidelines above. The CH2 domain is unique in that it is not closely paired with another domain. The CH2 domain is linked to the CH3 domain, and is also linked to the CH1 domain through a hinge region. This hinge region encompasses a variable number of amino acid residues which is on the order of 25 residues for IgG1, IgG2, and IgG4 but somewhat longer in IgG3. Importantly, the hinge region is flexible, thereby allowing the two N-terminal antigen binding regions to move independently. Antibodies which lack the CH2 domain will spontaneously assemble to form stable heterodimers or homodimers, held together through non-covalent interactions.

In CH2 deleted heavy chains, the CH3 domain may be linked directly to the hinge region of the respective heavy chains, or may be joined to the hinge region with an amino acid spacer. The spacer may be any convenient length, for example from about 1 to 20 amino acids in length, for example, about 1, about 2, about 3, about 4, about 5, about 6, about 7, about 8, about 9, about 10, about 11, about 12, about 13, about 14, about 15, about 16, about 17, about 18, about 19, about 20 or more amino acids.

One example of intracellular assembly and identification of bispecific tetravalent antibodies with one fixed and one variable specificity is shown in FIG. 1D. A library of polynucleotides encoding diverse first CH2 domain-deleted heavy chain subunit polypeptides is introduced into host cells together with (i) a library of polynucleotides encoding diverse light chain subunit polypeptides; (ii) a polynucleotide encoding a single fixed CH2 domain-deleted second heavy chain subunit polypeptide or polynucleotides encoding a plurality of defined CH2 domain-deleted second heavy chain subunit polypeptides, that contribute to a defined antigen specificity when paired with certain light chains, and optionally have been modified to express a first heterodimerization domain in its constant region; (iii) optionally, the same CH2 domain-deleted second heavy chain subunit polypeptide(s) that contribute to a defined antigen specificity as in (ii) which have been modified to express a complementing second heterodimerization domain in their constant region to that in (ii); and (iv) a polynucleotide encoding a single fixed light chain subunit polypeptide or polynucleotides encoding a plurality of defined light chain subunit polypeptides, that when paired with the defined heavy chain subunit polypeptide(s) of (ii) creates an antigen binding domain with a defined specificity. The optional heterodimerization domain(s) may be either a “protuberance” or a “cavity”. If a protuberance, the complementing heterodimerization domain is a cavity. If a cavity, the complementing heterodimerization domain is a protuberance. Similarly, if heterodimerization is promoted by a leucine zipper, the heterodimerization domain may be either Fos or Jun; if Fos, the complementing heterodimerization is Jun; if Jun, the complementing heterodimerization domain is Fos.

In a related embodiment, diagrammed in FIG. 1C, the library of polynucleotides encoding diverse immunoglobulin light chains is omitted in the method of the previous paragraph and the only light chain-encoding polynucleotides encode the defined light chain(s) which when paired with the defined heavy chain(s) create an antigen binding domain with the defined specificity. In one embodiment only a polynucleotide that encodes a single fixed light chain is introduced into host cells together with the polynucleotides that encode diverse first immunoglobulin heavy chains and the defined second heavy chain subunit polypeptides. This has the effect of forcing selection of bispecific tetravalent antibodies with two different antigen specificities, one variable and one pre-defined, that employ the same light chain.

In certain embodiments, the complexity of heavy and/or light chain polynucleotide libraries is reduced by prior selection of polynucleotides which encode monospecific bivalent antibodies that bind to a surface antigen of the target cell of interest, followed by incorporation of the variable regions of these isolated polynucleotides into sublibraries comprising polynucleotides that encode CH2 domain-deleted heavy chain constant regions with, optionally, heterodimerization domains for selection of bispecific tetravalent antibodies.

In one embodiment, if a library of polynucleotides encoding diverse light chain subunit polypeptides is included, then at the same time, prior to, or subsequent to identification of the polynucleotide(s) encoding one or more first heavy chain subunit polypeptides as above, polynucleotides encoding one or more light chain subunit polypeptides are identified which in combination with the first heavy chain subunit polypeptides and the defined heavy and light chain subunit polypeptides comprise bispecific tetravalent antibodies with a defined binding specificity or function. In subsequent steps, additional polynucleotides encoding immunoglobulin heavy and/or light chain polypeptides may be identified which when substituted for one of the initially selected heavy and/or light chain-encoding polynucleotides further enhance the ability to recognize specific antigens and/or perform a specific function.

The polynucleotides encoding heavy chain subunit polypeptides may be constructed for expression as either bispecific tetravalent membrane receptors or as secreted bispecific tetravalent antibody molecules which are identified through interaction with antigen or target cells for the desired specificity described elsewhere (US 20020123057A1, published Sep. 5, 2002).

In another embodiment, bispecific tetravalent antibodies are identified with two variable specificities. In this embodiment, two libraries of polynucleotides encoding diverse immunoglobulin CH2 domain-deleted heavy chains, optionally comprising complementary heavy chain heterodimerization domains, are introduced into host cells together with a single library of polynucleotides encoding diverse immunoglobulin light chains. As noted above, in certain embodiments the complexity of one or more of these libraries is reduced by first identifying monospecific bivalent antibodies that bind to a surface epitope of the target cell of interest and incorporating polynucleotides encoding the variable regions of these isolated polynucleotides into one light chain- and two heavy chain-encoding polynucleotide sublibraries, wherein the encoded heavy chains comprise CH2 domain-deleted constant regions and optionally complementary heavy chain heterodimerization domains for identification of bispecific tetravalent antibodies.

In certain other embodiments, bispecfic tetravalent antibodies may be assembled extracellularly. Polynucleotides are identified which encode two monospecific bivalent antibodies, one monospecific bivalent antibody and one bispecific bivalent antibody, or two bispecific bivalent antibodies as described herein and in WO 00/028016. Bispecific tetravalent antibodies are then assembled with two monospecific bivalent antibodies, one monospecific bivalent antibody and one bispecific bivalent antibody, or two bispecific bivalent antibodies with variable specificities or with one fixed and one variable specificity. The two monospecific bivalent antibodies, one monospecific bivalent antibody and one bispecific bivalent antibody, or two bispecific bivalent antibodies can be crosslinked to each other by various means for tetramerization described herein, and screened for antigen-binding and/or induction of a physiological response.

Various monospecific bivalent antibodies, monospecific bivalent antibodies and bispecific bivalent antibodies, or bispecific bivalent antibodies can be crosslinked, either to each other or to antibodies of known specificity, through any method known for crosslinking antibodies, including, but not limited to, physical and/or chemical crosslinking. For example, as described in WO 00/44788, a thiol-containing residue can be introduced into the constant region of the antibodies to permit formation of disulfide bonds between two bivalent antibodies. Preferably, the thiol-containing residue is incorporated at a site on the outside loop of a domain so as to minimize potential for intrachain disulfide bonds. Exemplary amino acid residues for replacing with thiol-containing residues on IgG heavy chains include, but are not limited to 416, 420 and 421.

Chemical cross-linking can be performed, for instance by a number of reagents including: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-α-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, formaldehyde and succinimidyl 4-[N-maleimidomethyl]cyclohexane-1-carboxylate.

In another embodiment, an immunoglobulin subunit polypeptide contains an amino acid sequence which is a recognition site for a modifying enzyme. Modifying enzymes include BirA, various glycosylases, farnesyl protein transferase, and protein kinases. The group introduced by the modifying enzyme, e.g. biotin, sugar, phosphate, farnesyl, etc. provides a complementary binding pair member, or a unique site for further modification, such as chemical cross-linking, biotinylation, etc. that will provide a complementary binding pair member.

The recognition site for the modifying enzyme may be naturally occurring, or may be introduced through genetic engineering. The site will be a specific binding pair member or one that is modified to provide a specific binding pair member, where the complementary pair has a multiplicity of specific binding sites. Binding to the complementary binding member can be a chemical reaction, epitope-receptor binding or hapten-receptor binding where a hapten is linked to the subunit chain.

In one embodiment, a bivalent, monospecific or bispecific antibody or fragment thereof can be linked to other antibodies via avidin either directly or indirectly. An immunoglobulin subunit polypeptide, for example, the constant region of the heavy chain, may be engineered to contain a site for biotinylation, for example a BirA-dependent site and multiple such antibodies may be linked by binding to avidin. Alternatively, direct linkage is accomplished by making an antibody-avidin fusion protein of one or more antibodies with fixed or variable specificity through genetic engineering as described in, for example, Shin, S.-U. et al., J. Immunol. 158:4797-4804 (1997); and Penichet et al., J. Immunol. 163:4421-4426 (1999). Combining the antibody avidin fusion protein with other biotinylated antibodies will result in assembly of higher order multivalent, multispecific complexes.

Alternatively, the immunoglobulin subunit polypeptides can be genetically modified by including sequences encoding amino acid residues with chemically reactive side chains such as Cys or His. Suitable side chains can be used to chemically link two or more monospecific bivalent antibodies, one or more monospecific bivalent antibodies and one or more bispecific bivalent antibodies, or two or more bispecific bivalent antibodies to a suitable dendrimer particle. Dendrimers are synthetic chemical polymers that can have any one of a number of different functional groups on their surface (D. Tomalia, Aldrichimica Acta 26:91:101 (1993)). Exemplary dendrimers for use in accordance with the present invention include e.g., E9 starburst polyamine dendrimer and E9 combburst polyamine dendrimer, which can link cysteine residues. The antibody molecules are modified to introduce a cysteine residue at the carboxyl terminus. Cysteine modified antibodies will react with the maleimide groups on the various peptide backbones with either two, three, or four modified lysine residues for formation of antibody dimers, trimers, and tetramers.

Alternatively, one or more monospecific bivalent antibodies, or one or more bispecific bivalent antibodies may be cross-linked to each other or to an antibody of known specificity through a third antibody. In one embodiment, the antibody of known specificity contains a constant region of one immunoglobulin class or subclass (e.g. IgG1), and the one or more monospecific bivalent antibodies, or one or more bispecific bivalent antibodies identified according to the present invention contain a constant region of another immunoglobulin class or subclass (e.g. IgG2, IgG3, IgG4 or IgA). The antibodies are cross-linked through a third antibody which is bispecific for the two classes or subclasses of antibody constant regions. In another embodiment, the antibody of known specificity contains a rodent constant region, and the one or more monospecific bivalent antibodies, or one or more bispecific bivalent antibodies identified according to the present invention contain a human constant region. The antibodies are cross-linked through a third antibody which is bispecific for human and rodent antibody constant regions.

In other embodiments, antigen binding domains, or subunits thereof, can be formed as fusion proteins. For example, a library of polynucleotides encoding diverse immunoglobulin heavy chains may be engineered to encode a fusion protein, where the heterologous region of the fusion protein comprises a fixed immunoglobulin heavy chain variable region, that when paired with a defined light chain, provided either exogenously or as part of the same fusion protein, creates an antigen binding domain with a known, desired specificity. Under this “means for tetramerization,” the library of polynucleotides encoding diverse heavy chains is introduced into host cells together with polynucleotides encoding either fixed or variable light chains much like a monospecific library to detect an antibody with a desired specificity. Identification of this specificity is carried out through detection of bispecific binding, to the known epitope by the fixed antigen binding domain, coupled with binding to a second unknown epitope, where the bispecific binding results in a detectable signal. Where the desired epitopes are on a target cell, a detectable signal would be, for example, cellular proliferation, functional activation, apoptosis, or differentiation.

FIG. 1C shows a bispecific tetravalent antibody comprising a single fixed light chain which confers a desired specificity when associated with a pre-selected, fixed heavy chain and which can also associate with a second randomized heavy chain to confer a different specificity in another arm of the tetravalent antibody. In host cells into which polynucleotides are introduced that encode one heavy chain of a library of diverse first immunoglobulin heavy chains with a CH2 domain deletion, along with a polynucleotide that encodes a fixed heavy chain with a CH2 domain deletion, and a polynucleotide that encodes a fixed light chain, individual CH2 domain-deleted IgG molecules spontaneously form non-covalently associated tetravalent complexes. In this system, 87.5% of the tetravalent antibodies are expected, on average, to be a productive combination in the sense of having at least one antigen combining site with a pre-determined specificity determined by the fixed heavy chain and one antigen combining site with a variable specificity determined by the randomized heavy chain.

FIG. 1D shows a bispecific tetravalent antibody comprised of a single fixed light chain which confers a desired specificity when associated with a pre-selected, fixed heavy chain; and a second randomized heavy chain which can associate with either the fixed light chain or a randomized light chain in another arm of the antibody. In host cells into which polynucleotides are introduced that encode one heavy chain of a library of diverse heavy chains with a CH2 domain deletion, along with a polynucleotide encoding one fixed heavy chain with a CH2 domain deletion, a polynucleotide encoding one fixed light chain, and polynucleotides encoding one second light chain from a library of diverse light chains, individual CH2 domain-deleted IgG molecules spontaneously form non-covalently associated tetravalent complexes. In this system, 43% of the antibodies are expected, on average, to be a productive combination in the sense of having at least one antigen combining site with a pre-determined specificity comprised of the fixed heavy and light chains and one antigen combining site with a variable specificity comprised of the randomized heavy and light chains.

Where immunoglobulin antigen binding domains are composed of one polypeptide, i.e., a single-chain fragment or a fragment comprising a V_{H}H domain, and therefore are encoded by one polynucleotide, preferred methods comprise a one-step screening and/or selection process for monospecific antibodies. Polynucleotides encoding a single-chain fragment, comprising a heavy chain variable region and a light chain variable region, or comprising a V_{H}H region, are identified from a library by introducing the library into host cells such as eukaryotic cells and recovering polynucleotides of said library from those host cells which encode immunoglobulin fragments which contribute to a desired specificity. Alternatively, bispecific antibodies comprised of two single-chain fragments or V_{H}H domains can be formed by introducing heterodimerization domains or recognition sites for a modifying enzyme as described above.

The multispecific antibodies of the invention may be used to cross-link heteromeric receptor complexes, e.g., on a target cell, either known or unknown, to promote a physiological response. Physiological responses include, but are not limited to apoptosis, cell proliferation, cell differentiation, or secretion of cytokines. Known heteromeric complexes which activate a physiological response include for example, heteromeric BMP complexes, LIFRα/gp130 complexes, and GFRα1/Ret complexes as described herein.

In addition, multispecific antibodies which contain binding sites for a antigenic determinant of pathogen e.g., a viral protein expressed on the surface of an infected cell, and at least one specificity for the HLA class II invariant chain (Ii) can be used to induce clearance of the pathogen. In addition to pathogens, clearance of therapeutic or diagnostic agents, autoantibodies, anti-graft antibodies, and other undesirable compounds may be induced using the multispecific antibodies, as described in U.S. Pat. No. 6,458,933.

Multispecific antibodies can also be used to deliver a therapeutic agent to a target cell. These types of mutlispecific antibodies have an antigen binding site for a therapeutic agent, and an antigen binding site for a surface marker of a target cell. The therapeutic agent can be a drug, toxin, enzyme, DNA, radionuclide, etc. The target cell can be an infected cell, cancerous cell, etc.

Where the immunoglobulin molecules are bound to the host cell surface, the first identification step comprises introducing into a population of host cells capable of expressing the immunoglobulin molecule a one or more libraries of polynucleotides encoding a plurality of first heavy chain subunit polypeptides comprising a transmembrane domain, through operable association with a transcriptional control region, or optionally, one library of heavy chain-encoding polynucleotides and a polynucleotide encoding a previously identified single fixed membrane bound immunoglobulin heavy chain, introducing into the same host cells a library of polynucleotides encoding, through operable association with a transcriptional control region, a plurality of light chain subunit polypeptides, or optionally, a polynucleotide encoding a previously identified, single fixed immunoglobulin light chain, permitting expression of immunoglobulin molecules, or antigen-binding fragments thereof, on the membrane surface of the host cells, contacting the host cells with an antigen or antigens, e.g., expressed on a target cell, and recovering polynucleotides derived from the first heavy chain library and optionally from the light chain library, from those host cells which bind to two non-identical epitopes of the antigen or antigens or which trigger a desired physiological effect in the interacting target cells.

Where the immunoglobulin molecules are fully secreted into the cell medium, the first identification step comprises introducing into a population of host cells capable of expressing the immunoglobulin molecule one or more libraries of polynucleotides encoding a plurality of heavy chain subunit polypeptides which are fully secreted, through operable association with a transcriptional control region, or optionally, one library of heavy chain polynucleotides and a polynucleotide encoding a previously identified single fixed secreted immunoglobulin heavy chain, introducing into the same host cells a library of polynucleotides encoding, through operable association with a transcriptional control region, a plurality of light chain subunit polypeptides, or optionally, a polynucleotide encoding a previously identified, single fixed immunoglobulin light chain, permitting expression and secretion of immunoglobulin molecules, or antigen-binding fragments thereof into the cell medium, assaying aliquots of conditioned medium for desired antigen-related antibody functions, e.g., on a target cell upon binding of the antibody to at least two non-identical epitopes, and recovering polynucleotides derived from the heavy chain library and optionally from the light chain library, from those host cell pools grown in conditioned medium in which the desired response in the target cell was observed.

In other embodiments, monospecific immunoglobulin molecules are first identified. The first identification step comprises introducing into a population of host cells capable of expressing the immunoglobulin molecule a first library of polynucleotides encoding a plurality of first immunoglobulin subunit polypeptides through operable association with a transcriptional control region, introducing into the same host cells a second library of polynucleotides encoding, through operable association with a transcriptional control region, a plurality of second immunoglobulin subunit polypeptides, permitting expression and secretion of immunoglobulin molecules, or fragments thereof, into the cell medium, cross-linking the immunoglobulin molecules or antigen-specific fragments thereof with an antibody of known specificity, assaying aliquots of conditioned medium for desired antigen-related antibody functions upon binding of the antibody to two non-identical epitopes, and recovering polynucleotides derived from the first library and from the second library from those host cell pools grown in conditioned medium in which the desired function was observed.

As used herein, a “library” is a representative genus of polynucleotides, i.e., a group of polynucleotides related through, for example, their origin from a single animal species, tissue type, organ, or cell type, where the library collectively comprises at least two different species within a given genus of polynucleotides. A library of polynucleotides preferably comprises at least 10, at least 100, at least 10³, at least 10⁴, at least 10⁵, at least 10⁶, at least 10⁷, at least 10⁸, or at least 10⁹ different species within a given genus of polynucleotides. More specifically, a library of the present invention encodes a plurality of a certain immunoglobulin subunit polypeptide, i.e., either a heavy chain subunit polypeptide or a light chain subunit polypeptide. In this context, a “library” of the present invention comprises polynucleotides of a common genus, the genus being polynucleotides encoding an immunoglobulin subunit polypeptide of a certain type and class e.g., a library might encode a human μ, γ-1, γ-2, γ-3, γ-4, α-1, α-2, ε, or δ heavy chain, or a human kappa or lambda light chain, where the various immunoglobulin subunit polypeptides may be modified, for example to contain a heterodimerization domain or to be a CH2-deleted heavy chain. Although each member of any one library of the present invention will encode the same heavy or light chain constant region (with the same modifications, if any), the library will collectively comprise at least two, at least 10, at least 100, at least 10³, at least 10⁴, at least 10⁵, at least 10⁶, at least 10⁷, at least 10⁸, or at least 10⁹ different variable regions i.e., a “plurality” of variable regions associated with the common constant region. For convenience, it may also be said that a library of polynucleotides encodes a corresponding library of immunoglobulin subunit polypeptides or fragments thereof, where such polypeptide libraries have the same number and categories of members as the polynucleotide library.

In other embodiments, the library encodes a plurality of immunoglobulin single-chain fragments which comprise a variable region, such as a light chain variable region or a heavy chain variable region, and may comprise both a light chain variable region and a heavy chain variable region. Optionally, such a library comprises polynucleotides encoding an immunoglobulin subunit polypeptide of a certain type and class, or domains thereof.

In one aspect, the present invention encompasses methods to produce libraries of polynucleotides encoding immunoglobulin subunit polypeptides suitable for inclusion in multispecific antibodies. Furthermore, the present invention encompasses libraries of immunoglobulin subunit polypeptides constructed in eukaryotic expression vectors according to the methods described herein. Such libraries may be produced in eukaryotic virus vectors, for example, a poxvirus vector such as vaccinia virus. Such methods and libraries are described herein.

By “recipient cell” or “host cell” is meant a cell or population of cells into which polynucleotide libraries of the present invention are introduced. In certain embodiments, a host cell of the present invention is a eukaryotic cell or cell line, for example, a plant, animal, vertebrate, mammalian, rodent, mouse, primate, or human cell or cell line. By “a population of host cells” is meant a group of cultured cells into which a “library” of the present invention can be introduced and expressed. Any host cells which will support expression from a given library constructed in a given vector is intended. Suitable host cells are disclosed herein. Furthermore, certain particular host cells for use with specific vectors and with specific selection and/or screening schemes are disclosed herein. Although a population of host cells is typically a monoculture, i.e., where each cell in the population is of the same cell type, mixed cultures of cells are also contemplated. Host cells of the present invention may be adherent, i.e., host cells which grow attached to a solid substrate, or, alternatively, the host cells may be in suspension. Host cells may be cells derived from primary tumors, cells derived from metastatic tumors, primary cells, cells which have lost contact inhibition, transformed primary cells, immortalized primary cells, cells which may undergo apoptosis, and cell lines derived therefrom.

As noted above, methods to identify immunoglobulin molecules comprise the introduction of one or more libraries of polynucleotides into a population of host cells, e.g., one or more libraries of polynucleotides encoding immunoglobulin heavy chains, and/or one or more libraries of polynucleotides encoding immunoglobulin light chains. Where two heavy chain libraries are employed, they are complementary, for example, they will encode heavy chains with complementary heterodimerization domains, thereby allowing assembly of multispecific antibodies, or antigen-binding fragments thereof, in the population of host cells. Also, as noted above, another method to identify antibodies or antibody fragments comprises introduction of a one or more libraries of polynucleotides encoding single-chain fragments into a population of host cells. The libraries may be constructed in any suitable vectors, and all libraries may, but need not be, constructed in the same vector. Suitable vectors for libraries of the present invention are disclosed infra.

Polynucleotides contained in libraries of the present invention encode immunoglobulin subunit polypeptides through “operable association with a transcriptional control region.” One or more nucleic acid molecules in a given polynucleotide are “operably associated” when they are placed into a functional relationship. This relationship can be between a coding region for a polypeptide and a regulatory sequence(s) which are connected in such a way as to permit expression of the coding region when the appropriate molecules (e.g., transcriptional activator proteins, polymerases, etc.) are bound to the regulatory sequences(s). “Transcriptional control regions” include, but are not limited to promoters, enhancers, operators, and transcription termination signals, and are included with the polynucleotide to direct its transcription. For example, a promoter would be operably associated with a nucleic acid molecule encoding an immunoglobulin subunit polypeptide if the promoter was capable of effecting transcription of that nucleic acid molecule. Generally, “operably associated” means that the DNA sequences are contiguous or closely connected in a polynucleotide. However, some transcription control regions, e.g., enhancers, do not have to be contiguous.

By “control sequences” or “control regions” is meant DNA sequences necessary for the expression of an operably associated coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A variety of transcriptional control regions are known to those skilled in the art. Preferred transcriptional control regions include those which function in vertebrate cells, such as, but not limited to, promoter and enhancer sequences from poxviruses, adenoviruses, herpesviruses, e.g., human cytomegalovirus (for example, the intermediate early promoter, preferably in conjunction with intron-A), alphaviruses, simian virus 40 (for example, the early promoter), retroviruses (such as Rous sarcoma virus), and picornaviruses (particularly an internal ribosome entry site, or IRES, enhancer region, also referred to herein as a CITE sequence). Other transcriptional control regions include those derived from mammalian genes such as actin, heat shock protein, and bovine growth hormone, as well as other sequences capable of controlling gene expression in eukaryotic cells. Additional suitable transcription control regions include tissue-specific promoters and enhancers as well as inducible promoters (e.g., promoters inducible by tetracycline, and temperature sensitive promoters). As will be discussed in more detail below, particular embodiments include promoters capable of functioning in the cytoplasm of poxvirus-infected cells.

In certain embodiments, each “immunoglobulin subunit polypeptide,” i.e., either a “first heavy chain subunit polypeptide,” a “second heavy chain subunit polypeptide,” or a “light chain subunit polypeptide” comprises (i) a first immunoglobulin constant region, either a heavy chain constant region, either a membrane bound form of a heavy chain constant region or a fully secreted form of a heavy chain constant region or a light chain constant region, (ii) an immunoglobulin variable region corresponding to the first constant region, i.e., if the immunoglobulin constant region is a heavy chain constant region, the immunoglobulin variable region preferably comprises a VH region, and if the immunoglobulin constant region is a light chain constant region, the immunoglobulin variable region preferably comprises a VL region, which may be either a V-kappa or a V-lambda region, and (iii) a signal peptide capable of directing transport of the immunoglobulin subunit polypeptide through the endoplasmic reticulum and through the host cell plasma membrane, either as a membrane-bound or fully secreted heavy chain, or a light chain associated with a heavy chain. Additional modifications to immunoglobulin subunit polypeptides are contemplated, e.g., the inclusion of a heterdimerization domain or a tetramerization domain contained in a heavy chain constant region. Through the association of two or more heavy chains and two or more light chains, either a surface immunoglobulin molecule or a fully secreted immunoglobulin molecule is formed. In addition, one or more immunoglobulin subunit polypeptides of any type discussed herein may be fused together such that two or more different variable regions contributing to two or more non-identical antigen binding domains may be included on a single subunit polypeptide.

Also in certain embodiments in the context of an immunoglobulin fragment, a single-chain fragment comprises an immunoglobulin variable region selected from the group consisting of a heavy chain variable region and a light chain variable region, and preferably comprises both variable regions. If the immunoglobulin fragment comprises both a heavy chain variable region and a light chain variable region, they may be directly joined (i.e., they have no peptide or other linker), or they may be joined by another means. If they are joined by other means, they may be joined directly or by a disulfide bond formed during expression or by a peptide linker, as discussed below. Accordingly, through the association of the heavy chain variable region and the light chain variable region, an antigen binding domain is formed.

The heavy chain variable region and light chain variable region of one single-chain fragment may associate with one another or the heavy chain variable region of one single-chain fragment may associate with a light chain variable region of another single-chain fragment, and vice versa, depending on the length of linker between heavy and light chain variable regions on each fragment. In one embodiment, the single-chain fragment also comprises a constant region selected from the group consisting of a heavy chain constant region, or a domain thereof, and a light chain constant region, or a domain thereof. Two single-chain fragments may associate with one another via their constant regions.

As mentioned above, in certain embodiments, the polynucleotide encoding the light chain variable region and heavy chain variable region of the single-chain fragment encode a linker. The single-chain fragment may comprise a single polypeptide with the sequence VH-linker-VL or VL-linker-VH. In some embodiments, the linker is chosen to permit the heavy chain and light chain of a single polypeptide to bind together in their proper conformational orientation. See for example, Huston, J. S., et al., Methods in Enzym. 203:46-121 (1991). Thus, in these embodiments, the linker should be able to span the 3.5 nm distance between its points of fusion to the variable domains without distortion of the native Fv conformation. In these embodiments, the amino acid residues constituting the linker are such that it can span this distance and should be 5 amino acids or longer. Single-chain fragments with a linker of 5 amino acids are found in monomer and predominantly dimer form. Preferably, the linker should be at least about 10 or at least about 15 residues in length. In other embodiments, the linker length is chosen to promote the formation of scFv tetramers (tetrabodies), and is 1 amino acid in length. In some embodiments, the variable regions are directly linked (i.e., the single-chain fragment contains no peptide linker) to promote the formation of scFv trimers (triabodies). These variations are well known in the art. (See, for example, Chames and Baty, FEMS Microbiol. Letts. 189:1-8 (2000)). The linker should not be so long it causes steric interference with the combining site. Thus, it preferably should be about 25 residues or less in length.

The amino acids of the peptide linker are preferably selected so that the linker is hydrophilic so it does not get buried into the antibody. The linker (Gly-Gly-Gly-Gly-Ser)₃ (SEQ ID NO:3) is a preferred linker that is widely applicable to many antibodies as it provides sufficient flexibility. Other linkers include Glu Ser Gly Arg Ser Gly Gly Gly Gly Ser Gly Gly Gly Gly Ser (SEQ ID NO:4), Glu Gly Lys Ser Ser Gly Ser Gly Ser Glu Ser Lys Ser Thr (SEQ ID NO:5), Glu Gly Lys Ser Ser Gly Ser Gly Ser Glu Ser Lys Ser Thr Gln (SEQ ID NO:6), Glu Gly Lys Ser Ser Gly Ser Gly Ser Glu Ser Lys Val Asp (SEQ ID NO:7), Gly Ser Thr Ser Gly Ser Gly Lys Ser Ser Glu Gly Lys Gly (SEQ ID NO:8), Lys Glu Ser Gly Ser Val Ser Ser Glu Gln Leu Ala Gln Phe Arg Ser Leu Asp (SEQ ID NO:9), and Glu Ser Gly Ser Val Ser Ser Glu Glu Leu Ala Phe Arg Ser Leu Asp (SEQ ID NO:10). Alternatively, a linker such as SEQ ID NO:3, although any sequence can be used, is mutagenized or the amino acids in the linker are randomized, and using phage display vectors or the methods of the invention, antibodies with different linkers are screened or selected for the highest affinity or greatest effect on phenotype. Examples of shorter linkers include fragments of the above linkers, and examples of longer linkers include combinations of the linkers above, combinations of fragments of the linkers above, and combinations of the linkers above with fragments of the linkers above.

Also preferred are immunoglobulin subunit polypeptides which are variants or fragments of the above-described immunoglobulin subunit polypeptides. Any variants or fragments which result in an antigen binding fragment of an immunoglobulin molecule are contemplated. Such variants may be attached to the host cell surface, e.g., through association with a naturally-occurring transmembrane domain, through a receptor-ligand interaction, or as a fusion with a heterologous transmembrane domain, or may be secreted into the cell medium. Examples of antigen binding fragments of immunoglobulin molecules are described herein.

In those embodiments where the immunoglobulin subunit polypeptide comprises a heavy chain polypeptide, any immunoglobulin heavy chain, from any animal species, is intended. Suitable immunoglobulin heavy chains are described herein. Immunoglobulin heavy chains from vertebrates such as birds, especially chickens, fish, and mammals are included. Examples of mammalian immunoglobulin heavy chains include human, mouse, dog, cat, horse, goat, rat, sheep, cow, pig, guinea pig, camel, llama, and hamster immunoglobulin heavy chains. Also contemplated are hybrid immunoglobulin heavy chains comprising portions of heavy chains from one or more species, such as mouse/human hybrid immunoglobulin heavy chains, or “camelized” human immunoglobulin heavy chains. Of the human immunoglobulin heavy chains, an immunoglobulin heavy chain of the present invention is selected from the group consisting of a μ heavy chain, i.e., the heavy chain of an IgM immunoglobulin, a γ-1 heavy chain, i.e., the heavy chain of an IgG1 immunoglobulin, a γ-2 heavy chain, i.e., the heavy chain of an IgG2 immunoglobulin, a γ-3 heavy chain, i.e., the heavy chain of an IgG3 immunoglobulin, a γ-4 heavy chain, i.e., the heavy chain of an IgG4 immunoglobulin, an α-1 heavy chain, i.e., the heavy chain of an IgA1 immunoglobulin, an α-2 heavy chain, i.e., the heavy chain of an IgA2 immunoglobulin, and ε heavy chain, i.e., the heavy chain of an IgE immunoglobulin, and a δ heavy chain, i.e., the heavy chain of an IgD immunoglobulin. Any of the above heavy chains may be modified so as to readily form bivalent or bispecific, tetravalent antibodies, e.g., to have a heterodimerization domain or a means for tetramerization, or so as to readily form multispecific tetravalent antibodies, e.g., a CH2-deleted constant domain.

Membrane bound forms of immunoglobulins are typically anchored to the surface of cells by a transmembrane domain which is made part of the heavy chain polypeptide through alternative transcription termination and splicing of the heavy chain messenger RNA. See, e.g., Roitt at page 9.10. By “transmembrane domain” “membrane spanning region,” or related terms, which are used interchangeably herein, is meant the portion of heavy chain polypeptide which is anchored into a cell membrane. Typical transmembrane domains comprise hydrophobic amino acids as discussed in more detail below. By “intracellular domain,” “cytoplasmic domain,” “cytosolic region,” or related terms, which are used interchangeably herein, is meant the portion of the polypeptide which is inside the cell, as opposed to those portions which are either anchored into the cell membrane or exposed on the surface of the cell. Membrane-bound forms of immunoglobulin heavy chain polypeptides typically comprise very short cytoplasmic domains of about three amino acids. A membrane-bound form of an immunoglobulin heavy chain polypeptide of the present invention preferably comprises the transmembrane and intracellular domains normally associated with that immunoglobulin heavy chain, e.g., the transmembrane and intracellular domains associated with μ and δ heavy chains in pre-B cells, or the transmembrane and intracellular domains associated with any of the immunoglobulin heavy chains in B-memory cells. However, it is also contemplated that heterologous transmembrane and intracellular domains could be associated with a given immunoglobulin heavy chain polypeptide, for example, the transmembrane and intracellular domains of a μ heavy chain could be associated with the extracellular portion of a γ heavy chain. Alternatively, transmembrane and/or cytoplasmic domains of an entirely heterologous polypeptide could be used, for example, the transmembrane and cytoplasmic domains of a major histocompatibility molecule, a cell surface receptor, a virus surface protein, chimeric domains, or synthetic domains.

In those embodiments where the immunoglobulin subunit polypeptide comprises a light chain polypeptide, any immunoglobulin light chain, from any animal species, is intended. Suitable immunoglobulin light chains are described herein. Immunoglobulin light chains from vertebrates such as birds, especially chickens, fish, and mammals are included. Examples of mammalian immunoglobulin light chains include human, mouse, dog, cat, horse, goat, rat, sheep, cow, pig, guinea pig, and hamster immunoglobulin light chains. Typically, light chains are either kappa light chains or lambda light chains. Also contemplated are hybrid immunoglobulin light chains comprising portions of light chains from one or more species, such as mouse/human hybrid immunoglobulin light chains or light chains comprising a kappa constant region and a lambda variable region, or vice versa. Two or more identical or non-identical light chains may associate with two or more identical or non-identical heavy chains to produce a multispecific antibodies (or antibody) as described herein.

According to one aspect of the invention, each member of a library of polynucleotides as described herein comprises (a) a first nucleic acid molecule encoding an immunoglobulin constant region common to all members of the library, and (b) a second nucleic acid molecule encoding an immunoglobulin variable region, where the second nucleic acid molecule is directly upstream of and in-frame with the first nucleic acid molecule. Accordingly, an immunoglobulin subunit polypeptide encoded by a member of a library of polynucleotides of the present invention, i.e., an immunoglobulin light chain or an immunoglobulin heavy chain encoded by such a polynucleotide, comprises an immunoglobulin constant region associated with an immunoglobulin variable region.

The constant region of a light chain comprises about half of the subunit polypeptide and is situated C-terminal, i.e., in the latter half of the light chain polypeptide. A light chain constant region, referred to herein as a C_{L} constant region, or, more specifically a C-kappa constant region or a C-lambda constant region, comprises about 110 amino acids held together in a “loop” by an intrachain disulfide bond.

The constant region of a heavy chain comprises about one-half to three quarters or more of the subunit polypeptide, depending, e.g., on modifications, and is situated in the C-terminal, i.e., in the latter portion of the heavy chain polypeptide. The heavy chain constant region, referred herein as a C_{H} constant region, comprises one or more, for example, one, two, three or four peptide loops or “domains” of about 110 amino acids each stabilized by intrachain disulfide bonds. More specifically, the heavy chain constant regions in human immunoglobulins include at least a portion of a Cμ constant region, a Cδ constant region, a Cγ constant region, a Cα constant region, or a Cε constant region. Full-size Cγ, Cα, and Cδ heavy chains each contain three constant region domains, referred to generally as C_{H}1, C_{H}2, and C_{H}3, while Cμ and Cε heavy chains contain four constant region domains, referred to generally as C_{H}1, C_{H}2, C_{H}3, and C_{H}4. In certain constant regions of the present invention, some or all of the C_{H}2 domain is deleted. In certain other constant regions of the present invention, the C_{H}3 domain, or other domain, is modified to comprise a heterodimerization domain.

Nucleic acid molecules encoding human immunoglobulin constant regions are readily obtained from cDNA libraries derived from, for example, human B cells or their precursors by methods such as PCR, which are well known to those of ordinary skill in the art and further, are disclosed in the Examples, infra. As discussed herein, the constant region of the heavy chain may be altered so as to preferentially form bispecific bivalent antibodies or bispecific tetravalent antibodies.

Immunoglobulin subunit polypeptides of the present invention each comprise an immunoglobulin variable region. Within a library of polynucleotides, each polynucleotide will comprise the same constant region, but the library will contain a plurality, i.e., at least two, at least 10, at least 100, at least 10³, at least 10⁴, at least 10⁵, at least 10⁶, at least 10⁷, at least 10⁸, or at least 10⁹ different variable regions. As is well known by those of ordinary skill in the art, a light chain variable region is encoded by rearranged nucleic acid molecules, each comprising a light chain VL region, specifically a V-Kappa region or a V-Lambda region, and a light chain J region, specifically a J-Kappa region or a J-Lambda region. Similarly, a heavy chain variable region is encoded by rearranged nucleic acid molecules, each comprising a heavy chain VH region, a D region and J region. These rearrangements take place at the DNA level upon cellular differentiation. Nucleic acid molecules encoding heavy and light chain variable regions may be derived, for example, by PCR from mature B cells and plasma cells which have terminally differentiated to express an antibody with specificity for a particular epitope. Furthermore, if antibodies to a specific antigen are desired, variable regions may be isolated from mature B cells and plasma cells of an animal that has been immunized with that antigen, and has thereby produced an expanded repertoire of antibody variable regions which interact with the antigen. Alternatively, if a more diverse library is desired, variable regions may be isolated from precursor cells, e.g., pre-B cells and immature B cells, which have undergone rearrangement of the immunoglobulin genes, but have not been exposed to antigen, either self or non-self. For example, variable regions might be isolated by PCR from normal human bone marrow pooled from multiple donors. In another embodiment, randomly diversified variable regions may be derived by PCR from centroblasts or centrocytes which have undergone somatic mutation in a germinal center. See, e.g., U.S. patent application Ser. No. 10/465,808, to Zauderer et al., filed Jun. 20, 2003, which is incorporated herein by reference in its entirety. Alternatively, variable regions may be synthetic, for example, made in the laboratory through generation of synthetic oligonucleotides, or may be derived through in vitro manipulations of germ line DNA resulting in rearrangements of the immunoglobulin genes.

In addition to nucleic acid molecules encoding immunoglobulin constant regions and variable regions, respectively, each member of a library of polynucleotides of the present invention as described above may further comprise a third nucleic acid molecule encoding a signal peptide directly upstream of and in frame with the nucleic acid molecule encoding the variable region.

By “signal peptide” is meant a polypeptide sequence which, for example, directs transport of nascent immunoglobulin polypeptide subunit to the membranes or exterior of the host cells. Signal peptides are also referred to in the art as “signal sequences,” “leader sequences,” “secretory signal peptides,” or “secretory signal sequences.” Signal peptides are normally expressed as part of a complete or “immature” polypeptide, and are normally situated at the N-terminus. The common structure of signal peptides from various proteins is commonly described as a positively charged n-region, followed by a hydrophobic h-region and a neutral but polar c-region. In many instances the amino acids comprising the signal peptide are cleaved off the protein once its final destination has been reached, to produce a “mature” form of the polypeptide. The cleavage is catalyzed by enzymes known as signal peptidases. The (−3,−1)-rule states that the residues at positions −3 and −1 (relative to the cleavage site) must be small and neutral for cleavage to occur correctly. See, e.g., McGeoch, Virus Res. 3:271-286 (1985), and von Heinje, Nucleic Acids Res. 14:4683-4690 (1986).

All cells, including host cells of the present invention, possess a constitutive secretory pathway, where proteins, including secreted immunoglobulin subunit polypeptides destined for export, are secreted from the cell. These proteins pass through the ER-Golgi processing pathway where modifications may occur. If no further signals are detected on the protein it is directed to the cells surface for secretion. Alternatively, immunoglobulin subunit polypeptides can end up as integral membrane components expressed on the surface of the host cells. Membrane-bound forms of immunoglobulin subunit polypeptides initially follow the same pathway as the secreted forms, passing through to the ER lumen, except that they are retained in the ER membrane by the presence of stop-transfer signals, or “transmembrane domains.” Transmembrane domains are hydrophobic stretches of about 20 amino acid residues that adopt an alpha-helical conformation as they transverse the membrane. Membrane embedded proteins are anchored in the phospholipid bilayer of the plasma membrane. As with secreted proteins, the N-terminal region of transmembrane proteins have a signal peptide that passes through the membrane and is cleaved upon exiting into the lumen of the ER. Transmembrane forms of immunoglobulin heavy chain polypeptides utilize the same signal peptide as the secreted forms.

A signal peptide of the present invention may be either a naturally-occurring immunoglobulin signal peptide, i.e., encoded by a sequence which is part of a naturally occurring heavy or light chain transcript, or a functional derivative of that sequence that retains the ability to direct the secretion of the immunoglobulin subunit polypeptide that is operably associated with it. Alternatively, a heterologous signal peptide, or a functional derivative thereof, may be used. For example, a naturally-occurring immunoglobulin subunit polypeptide signal peptide may be substituted with the signal peptide of human tissue plasminogen activator or mouse glucuronidase.

Signal sequences, transmembrane domains, and cytosolic domains are known for a wide variety of membrane bound proteins. These sequences may be used accordingly, either together as pairs (e.g., signal sequence and transmembrane domain, or signal sequence and cytosolic domain, or transmembrane domain and cytosolic domain) or threesomes from a particular protein, or with each component being taken from a different protein, or alternatively, the sequences may be synthetic, and derived entirely from consensus sequences as artificial delivery domains, as mentioned above.

Signal sequences and transmembrane domains include, but are not limited to, those derived from CD8, ICAM-2, IL-8R, CD4 and LFA-1. Additional useful sequences include sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al., Eur. J. Biochem. 174:671 (1988)) and insulin receptor beta-chain (residues 1-27 are the signal, 957-959, are the transmembrane domain and 960-1382 are the cytoplasmic domain; see Hatakeyama supra, and Ebina et al., Cell 40:747 (1985)); 2) class II integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). In this alternative, CD8 and ICAM-2 are particularly preferred. For example, the signal sequences from CD8 and ICAM-2 lie at the extreme 5′ end of the transcript. These consist of the amino acids 1-32 in the case of CD8 (Nakauchi et al., Proc. Natl. Acad. Sci. USA 82:5126 (1985)) and 1-21 in the case of ICAM-2 (Staunton et al., Nature (London) 339:61 (1989)). The transmembrane domains are encompassed by amino acids 145-195 from CD8 (Nakauchi, supra) and 224-256 from ICAM-2 (Staunton, supra).

Alternatively, membrane anchoring domains include the GPI anchor, which results in a covalent bond between the molecule and the lipid bilayer via a glycosyl-phosphatidylinositol bond for example in DAF (see Homans et al., Nature 333(6170):269-72 (1988), and Moran et al., J. Biol. Chem. 266:1250 (1991)). In order to do this, the GPI sequence from Thy-1 can be cassetted 3′ of the immunoglobulin or immunoglobulin fragment in place of a transmembrane sequence.

Similarly, myristylation sequences can serve as membrane anchoring domains. It is known that the myristylation of c-src recruits it to the plasma membrane. This is a simple and effective method of membrane localization, given that the first 14 amino acids of the protein are solely responsible for this function (see Cross et al., Mol. Cell. Biol. 4(9):1834 (1984); Spencer et al., Science 262:1019 1024 (1993)). This motif has already been shown to be effective in the localization of reporter genes and can be used to anchor the zeta chain of the TCR. This motif is placed 5′ of the immunoglobulin or immunoglobulin fragment in order to localize the construct to the plasma membrane. Other modifications such as palmitoylation can be used to anchor constructs in the plasma membrane; for example, palmitoylation sequences from the G protein-coupled receptor kinase GRK6 sequence (Stoffel et al, J. Biol. Chem 269:27791 (1994)); from rhodopsin (Barnstable et al., J. Mol. Neurosci. 5(3):207 (1994)); and the p21H-ras 1 protein (Capon et al., Nature 302:33 (1983)).

In addition to nucleic acid molecules encoding immunoglobulin constant regions and variable regions, respectively, each member of a library of polynucleotides of the present invention as described above may further comprise additional nucleic acid molecule encoding heterologous polypeptides. Such additional polynucleotides may be in addition to or as an alternative of the third nucleic acid molecule encoding a signal peptide. Such additional nucleic acid molecules encoding heterologous polypeptides may be upstream of or downstream from the nucleic acid molecules encoding the variable chain region or the heavy chain region. In certain embodiments, a heterologous polypeptide is an additional immunoglobulin subunit polypeptide.

A heterologous polypeptide encoded by an additional nucleic acid molecule may be a rescue sequence. A rescue sequence is a sequence which may be used to purify or isolate either the immunoglobulin or fragment thereof or the polynucleotide encoding it. Thus, for example, peptide rescue sequences include purification sequences such as the 6-His tag for use with Ni affinity columns and epitope tags for detection, immunoprecipitation, or FACS (fluorescence-activated cell sorting). Suitable epitope tags include myc (for use with commercially available 9E10 antibody), the BSP biotinylation target sequence of the bacterial enzyme BirA, flu tags, LacZ, and GST. The additional nucleic acid molecule may also encode a peptide linker.

In one embodiment, combinations of heterologous polypeptides are used. Thus, for example, any number of combinations of signal sequences, rescue sequences, and stability sequences may be used, with or without linker sequences. One can cassette in various fusion polynucleotides encoding heterologous polypeptides 5′ and 3 of the immunoglobulin or fragment thereof-encoding polynucleotide. As will be appreciated by those in the art, these modules of sequences can be used in a large number of combinations and variations.

The polynucleotides comprised in immunoglobulin subunit polypeptide libraries of the present invention are introduced into suitable host cells. Suitable host cells are characterized by being capable of expressing immunoglobulin molecules. Polynucleotides may be introduced into host cells by methods which are well known to those of ordinary skill in the art. Suitable introduction methods are disclosed herein.

As is easily appreciated, introduction methods vary depending on the nature of the vector in which the polynucleotide libraries are constructed. For example, DNA plasmid vectors may be introduced into host cells, for example, by lipofection (such as with anionic liposomes (see, e.g., Felgner et al., 1987 Proc. Natl. Acad. Sci. U.S. 84:7413 or cationic liposomes (see, e.g., Brigham, K. L. et al. Am. J. Med Sci. 298(4):278-2821(1989); U.S. Pat. No. 4,897,355 (Eppstein, et al.)), by electroporation, by calcium phosphate precipitation (see generally, Sambrook et al., Molecular Cloning: A Laboratory Manual, 2nd Ed., Cold Spring Harbor Laboratory, Cold Spring Harbor, N.Y., 1989), by protoplast fusion, by spheroplast fusion, or by the DEAE dextran method (Sussman et al., Cell. Biol. 4:1641-1643 (1984)). The above references are incorporated herein by reference in their entireties.

When the selected method is lipofection, the nucleic acid can be complexed with a cationic liposome, such as DOTMA:DOPE, DOTMA, DOPE, DC-cholesterol, DOTAP, Transfectam® (Promega), Tfx® (Promega), LipoTAXI™ (Stratagene), PerFect Lipid™ (Invitrogen), SuperFect™ (Qiagen). When the nucleic acid is transfected via an anionic liposome, the anionic liposome can encapsulate the nucleic acid. Preferably, DNA is introduced by liposome-mediated transfection using the manufacturer's protocol (such as for Lipofectamine; Life Technologies Incorporated).

Where the vector is a virus vector, introduction into host cells is most conveniently carried out by standard infection. However, in many cases viral nucleic acids may be introduced into cells by any of the methods described above, and the viral nucleic acid is “infectious,” i.e., introduction of the viral nucleic acid into the cell, without more, is sufficient to allow the cell to produce viable progeny virus particles. It is noted, however, that certain virus nucleic acids, for example, poxvirus nucleic acids, are not infectious, and therefore must be introduced with additional elements provided, for example, by a virus particle enclosing the viral nucleic acid, by a cell which has been engineered to produce required viral elements, or by a helper virus.

The libraries of polynucleotides encoding various immunoglobulin subunit polypeptides as described herein may be introduced into host cells in any order, or simultaneously, as may any polynucleotides encoding previously identified fixed immunoglobulin subunit polypeptides. For example, if one or more libraries of polynucleotides encoding heavy chains and/or light chains are constructed in virus vectors, whether infectious or inactivated, the vectors may be introduced by simultaneous infection as a mixture, or may be introduced in consecutive infections. If certain libraries are constructed in virus vectors, and others are constructed in plasmid vectors, introduction might be carried out most conveniently by introduction of one library before the other, preferably, by infection with the viral vector followed by transfection with the plasmid vector.

Following introduction into the host cells of the libraries of polynucleotides and optionally polynucleotides encoding previously identified immunoglobulin subunit polypeptides, expression of multispecific antibodies, or antigen-binding fragments thereof, is permitted to occur either on the membrane surface of the host cells, or through secretion into the cell medium. By “permitting expression” is meant allowing the vectors which have been introduced into the host cells to undergo transcription and translation of the immunoglobulin subunit polypeptides, preferably allowing the host cells to transport fully assembled immunoglobulin molecules, or antigen-binding fragments thereof, to the membrane surface or into the cell medium. Typically, permitting expression requires incubating the host cells into which the polynucleotides have been introduced under suitable conditions to allow expression. Those conditions, and the time required to allow expression will vary based on the choice of host cell and the choice of vectors, as is well known by those of ordinary skill in the art.

In certain embodiments, when the host cells secrete monospecific immunoglobulin molecules, the monospecific immunoglobulin molecules are cross-linked to antibodies of known specificity to form multispecific antibodies.

In certain embodiments, host cells which have been allowed to express immunoglobulin molecules on their surface, or soluble immunoglobulin molecules secreted into the cell medium are then contacted with an antigen or antigens. As used herein, an “antigen” includes one antigen or two or more antigens, and is any molecule that can specifically bind to an antibody, immunoglobulin molecule, or antigen-binding fragment thereof. By “specifically bind” is meant that the antigen binds to the CDR or “antigen binding domains” of the antibody. Thus, an “antigen-binding fragment” of an immunoglobulin molecule, typically comprising a heavy chain variable region and a light chain variable region, contains CDRs capable of specifically interacting with antigen. The portion of an antigen which specifically interacts with the CDR is an “epitope,” or an “antigenic determinant.” An antigen may comprise a single epitope, but typically, an antigen comprises at least two epitopes, and can include any number of epitopes, depending on the size, conformation, and type of antigen. A bispecific antibody of the present invention binds to two non-identical epitopes. The non-identical epitopes may be situated on a single antigen, or alternatively, may be situated on two separate antigens.

Antigens are typically peptides or polypeptides, but can be any molecule or compound. For example, an organic compound, e.g., dinitrophenol or DNP, a nucleic acid, a carbohydrate, or a mixture of any of these compounds either with or without a peptide or polypeptide can be a suitable antigen. The minimum size of a peptide or polypeptide epitope is thought to be about four to five amino acids. Peptide or polypeptide epitopes preferably contain at least seven, more preferably at least nine and most preferably between at least about 10 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitopes contain a sequence of at least 4, at least 5, at least 6, at least 7, at least 8, at least 9, at least 10, at least 15, at least 20, at least 25, or between about 15 to about 30 amino acids. Peptides or polypeptides comprising, or alternatively consisting of, epitopes are at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acid residues in length. The antigen may be in any form and may be free, for example dissolved in a solution, or may be attached to any substrate. Suitable substrates are disclosed herein. In certain embodiments, one or more antigens having one or more epitopes which are bound by a bispecific antibody of the present invention may be part of an antigen-expressing presenting cell or “target cell” as described in more detail below.

It is to be understood that immunoglobulin molecules specific for any antigen may be produced according to the methods of the present invention. In certain cases, antigens are “self” antigens, i.e., antigens derived from the same species as the immunoglobulin molecules produced. As an example, it might be desired to produce human antibodies directed to human tumor antigens such as, but not limited to, a CEA antigen, a GM2 antigen, a Tn antigen, an sTn antigen, a Thompson-Friedenreich antigen (TF), a Globo H antigen, an Le(y) antigen, a MUC1 antigen, a MUC2 antigen, a MUC3 antigen, a MUC4 antigen, a MUC5AC antigen, a MUC5B antigen, a MUC7 antigen, a carcinoembryonic antigen, a beta chain of human chorionic gonadotropin (hCG beta) antigen, a HER2/neu antigen, a PSMA antigen, a EGFRvIII antigen, a KSA antigen, a PSA antigen, a PSCA antigen, a GP100 antigen, a MAGE 1 antigen, a MAGE 2 antigen, a TRP 1 antigen, a TRP 2 antigen, and a tyrosinase antigen. Other desired “self” antigens include, but are not limited to, cytokine receptors, chemokine receptors, growth factor receptors, hormone receptors, and, more generally, surface membrane expressed proteins and glycoproteins that are singly or in concert with other surface membrane expressed molecules involved in mediating physiological responses of the cell. Physiological responses of interest include, but are not limited to, apoptosis, growth, and differentiation.

It is also contemplated to produce antibodies directed to antigens on infectious agents. Examples of such antigens include, but are not limited to, bacterial antigens, viral antigens, parasite antigens, and fungal antigens. Such antigens include antigens of infectious agents that are expressed or presented on the surface of an infected cell.

Examples of viral antigens include, but are not limited to, adenovirus antigens, alphavirus antigens, calicivirus antigens, e.g., a calicivirus capsid antigen, coronavirus antigens, distemper virus antigens, Ebola virus antigens, enterovirus antigens, flavivirus antigens, hepatitis virus (A-E) antigens, e.g., a hepatitis B core or surface antigen, herpesvirus antigens, e.g., a herpes simplex virus or varicella zoster virus glycoprotein antigen, immunodeficiency virus antigens, e.g., a human immunodeficiency virus envelope or protease antigen, infectious peritonitis virus antigens, influenza virus antigens, e.g., an influenza A hemagglutinin or neuramimidase antigen, leukemia virus antigens, Marburg virus antigens, oncogenic virus antigens, orthomyxovirus antigens, papilloma virus antigens, parainfluenza virus antigens, e.g., hemagglutinin/neuramimidase antigens, paramyxovirus antigens, parvovirus antigens, pestivirus antigens, picoma virus antigens, e.g., a poliovirus capsid antigen, rabies virus antigens, e.g., a rabies virus glycoprotein G antigen, reovirus antigens, retrovirus antigens, rotavirus antigens, SARS coronavirus antigens, as well as other cancer-causing or cancer-related virus antigens.

Examples of bacterial antigens include, but are not limited to, Actinomyces, antigens Bacillus antigens, Bacteroides antigens, Bordetella antigens, Bartonella antigens, Borrelia antigens, e.g., a B. bergdorferi OspA antigen, Brucella antigens, Campylobacter antigens, Capnocytophaga antigens, Chlamydia antigens, Clostridium antigens, Corynebacterium antigens, Coxiella antigens, Dermatophilus antigens, Enterococcus antigens, Ehrlichia antigens, Escherichia antigens, Francisella antigens, Fusobacterium antigens, Haemobartonella antigens, Haemophilus antigens, e.g., H. influenzae type b outer membrane protein antigens, Helicobacter antigens, Klebsiella antigens, L-form bacteria antigens, Leptospira antigens, Listeria antigens, Mycobacteria antigens, Mycoplasma antigens, Neisseria antigens, Neorickettsia antigens, Nocardia antigens, Pasteurella antigens, Peptococcus antigens, Peptostreptococcus antigens, Pneumococcus antigens, Proteus antigens, Pseudomonas antigens, Rickettsia antigens, Rochalimaea antigens, Salmonella antigens, Shigella antigens, Staphylococcus antigens, Streptococcus antigens, e.g., S. pyogenes M protein antigens, Treponema antigens, and Yersinia antigens, e.g., Y pestis F1 and V antigens.

Examples of fungal antigens include, but are not limited to, Absidia antigens, Acremonium antigens, Alternaria antigens, Aspergillus antigens, Basidiobolus antigens, Bipolaris antigens, Blastomyces antigens, Candida antigens, Coccidioides antigens, Conidiobolus antigens, Cryptococcus antigens, Curvalaria antigens, Epidermophyton antigens, Exophiala antigens, Geotrichum antigens, Histoplasma antigens, Madurella antigens, Malassezia antigens, Microsporum antigens, Moniliella antigens, Mortierella antigens, Mucor antigens, Paecilomyces antigens, Penicillium antigens, Phialemonium antigens, Phialophora antigens, Prototheca antigens, Pseudallescheria antigens, Pseudomicrodochium antigens, Pythium antigens, Rhinosporidium antigens, Rhizopus antigens, Scolecobasidium antigens, Sporothrix antigens, Stemphylium antigens, Trichophyton antigens, Trichosporon antigens, and Xylohypha antigens.

Examples of protozoan parasite antigens include, but are not limited to, Babesia antigens, Balantidium antigens, Besnoitia antigens, Cryptosporidium antigens, Eimeri antigens a antigens, Encephalitozoon antigens, Entamoeba antigens, Giardia antigens, Hammondia antigens, Hepatozoon antigens, Isospora antigens, Leishmania antigens, Microsporidia antigens, Neospora antigens, Nosema antigens, Pentatrichomonas antigens, Plasmodium antigens, e.g., P. falciparum circumsporozoite (PfCSP), sporozoite surface protein 2 (PfSSP2), carboxyl terminus of liver state antigen 1 (PfLSA-1 c-term), and exported protein 1 (PfExp-1) antigens, Pneumocystis antigens, Sarcocystis antigens, Schistosoma antigens, Theileria antigens, Toxoplasma antigens, and Trypanosoma antigens. Examples of helminth parasite antigens include, but are not limited to, Acanthocheilonema antigens, Aelurostrongylus antigens, Ancylostoma antigens, Angiostrongylus antigens, Ascaris antigens, Brugia antigens, Bunostomum antigens, Capillaria antigens, Chabertia antigens, Cooperia antigens, Crenosoma antigens, Dictyocaulus antigens, Dioctophyme antigens, Dipetalonema antigens, Diphyllobothrium antigens, Diplydium antigens, Dirofilaria antigens, Dracunculus antigens, Enterobius antigens, Filaroides, antigens Haemonchus antigens, Lagochilascaris antigens, Loa antigens, Mansonella antigens, Muellerius antigens, Nanophyetus antigens, Necator antigens, Nematodirus antigens, Oesophagostomum antigens, Onchocerca antigens, Opisthorchis antigens, Ostertagia antigens, Parafilaria antigens, Paragonimus antigens, Parascaris antigens, Physaloptera antigens, Protostrongylus antigens, Setaria antigens, Spirocerca, antigens Spirometra antigens, Stephanofilaria antigens, Strongyloides antigens, Strongylus antigens, Thelazia antigens, Toxascaris antigens, Toxocara antigens, Trichinella antigens, Trichostrongylus antigens, Trichuris antigens. Uncinaria antigens, and Wuchereria antigens.

Bispecific antibodies of the present invention may be identified and isolated by their ability to bind to bispecific receptors and activate receptor-mediated signalling in a target cell expressing the receptor. Many therapeutic uses for such bispecific antibodies are contemplated, including without limitation bispecific antibodies which bind to BMP receptors which may be used, e.g., to promote bone healing, bispecific antibodies which bind the LIFα/gp130 receptor complex for LIF which may be used, e.g., to facilitate endometrial implantation of embryos, and bispecific antibodies which bind to the GFRα/Ret receptor complex of GDNF which may be used, e.g., to facilitate regeneration of sensory axons after spinal cord injury.

While bispecific antibodies can be used to mimic ligand and therefore activate innate signaling, there are additional therapeutic uses that require the identification of bispecific antibodies which bind at least one unknown epitope. Such antibodies might be useful, for example, to facilitate a desired cellular activation through as yet undefined cell surface membrane components that may not be part of a natural ligand induced complex. Thus, the methods of identifying bispecific antibodies as described herein may be used to treat cellular disorders involving physiological activation mediated by cell surface membrane components, even where a specific ligand is not known.

For receptor tyrosine kinases whose targets are defined by proximity, for example, one need only create a bispecific antibody that produces a stable interaction between receptor components. Where the proximity is normally achieved through ligand binding to the receptor complex, a bispecific antibody may be able to use motifs outside of the ligand binding domain to achieve the same effect. Bispecific antibodies are a powerful tool to generate novel combinations of host cell proteins that trigger a desired response such as growth, functional activation, differentiation or apoptosis.

Polynucleotides encoding the various heavy and light chains which comprise a bispecific antibody are conveniently identified sequentially by screening various libraries of polynucleotides encoding various immunoglobulin subunit polypeptides sequentially in one or several “identification steps.” According to one sequential method, a library of polynucleotides encoding either immunoglobulin heavy or light chains is screened by starting with one or more heavy and/or light chains of a known, fixed specificity, and then screening the library for polynucleotides encoding immunoglobulin subunit polypeptides which combine with the subunit polypeptides of fixed specificity to form a desired bispecific antibody. Alternatively, two or more libraries of polynucleotides may be utilized in the first identification step, where one of the libraries is in a form in which the polynucleotides which encode one family of immunoglobulin subunit polypeptides (e.g., heavy chains) are readily recoverable, while another one of the libraries (e.g., a library which encodes light chains) is in a form where the immunoglobulin subunit polypeptides are expressed to allow assembly of bispecific antibodies, but the polynucleotides encoding those polypeptides are not readily recoverable.

As an alternative to sequential identification, two or more libraries of polynucleotides encoding different immunoglobulin subunit polypeptides may be identified at the same time by having the polynucleotides in both libraries be in recoverable form. One of ordinary skill in the art will readily understand how one or more of the “sequential” identification steps described herein can be performed simultaneously.

In a typical “first identification step,” bispecific antibodies of the present invention are expressed such that they are fully secreted from host cells, and are screened for by their ability to bind one or more antigens of interest. As discussed in more detail elsewhere herein, pools of host cells comprising libraries of polynucleotides which encode various subunit polypeptides of bispecific antibodies of the present invention are cultured under conditions permitting expression of bispecific antibody libraries. The medium in which the host cells are cultured, i.e., “conditioned medium,” is “contacted” with antigen by a method which will allow detection of the antigen-antibody interaction. For example, bispecific antibodies of interest in conditioned medium pools may be identified by their ability to bind to bispecific receptors on a target or indicator cell, thereby eliciting a detectable signal, e.g., apoptosis, proliferation, production of a cytokine, or differentiation. Other standard detection methods include, but are not limited to, immunoblots, ELISA assays, RIA assays, RAST assays, and immunofluorescence assays.

Other assays include, but are not limited to, virus neutralization assays (for antibodies directed to specific viruses), bacterial opsonization/phagocytosis assays (for antibodies directed to specific bacteria), antibody-dependent cellular cytotoxicity (ADCC) assays, assays to detect inhibition or facilitation of certain cellular functions, assays to detect IgE-mediated histamine release from mast cells, assays to detect apoptosis, assays to detect proliferation, assays to detect differentiation, hemagglutination assays, and hemagglutination inhibition assays. Such assays will allow detection of bispecific antibodies with desired functional characteristics or which elicit a desired response.

After the identification of conditioned medium pools containing bispecific antibodies which specifically bind antigens of interest, polynucleotides encoding bispecific antibodies are recovered from those pools, and further rounds of enrichment are carried out on the subset of polynucleotides expressed in host cells that produced the antibodies of interest until conditioned medium pools can be identified that are produced by host cells that express a less diverse and, therefore, better defined subset of polynucleotides.

By “recovery” is meant a crude separation of a desired component from those components which are not desired. For example, a subpool of host cells in which a desired antigen binding is detected are “recovered” based on their separation from other host cell pools, and polynucleotides of the library or libraries being screened are “recovered” from those cells by crude separation from other cellular components.

It is to be noted that the term “recovery” does not imply any sort of purification or isolation away from viral and other components. Recovery of polynucleotides may be accomplished by any standard method known to those of ordinary skill in the art. In a preferred aspect, the polynucleotides are recovered by harvesting infectious virus particles from a pool of conditioned medium in which a signal was detected, for example, particles of a vaccinia virus vector into which a given library has been constructed, which were contained in the pool of host cells in which antigen binding was detected.

As will be readily appreciated by those of ordinary skill in the art, identification of polynucleotides encoding immunoglobulin subunit polypeptides of a bispecific antibody in any given “identification step” will likely require two or more rounds of identification as described herein. Screening assays described herein identify pools containing the reactive host cells, and/or immunoglobulin molecules, but such pools will also contain non-reactive species. A single round of screening or selection may not necessarily result in recovery of a pure set of polynucleotides encoding the desired immunoglobulin subunit polypeptides; the mixture obtained after a first round may be enriched for the desired polynucleotides but may also be contaminated with non-desired insert sequences. Therefore, the reactive pools are further fractionated and subjected to further rounds of screening. Thus, in a first or subsequent identification step, identification of polynucleotides encoding the various immunoglobulin subunit polypeptides capable of forming a desired bispecific antibody, or antigen-binding fragment thereof, may require or benefit from several rounds of selection and/or screening, which thus increases the proportion of cells containing the desired polynucleotides. Accordingly, the polynucleotides recovered after the first round be introduced into a second population of cells and be subjected to a second round, a third round, a fourth round, or more rounds of enrichment, i.e., selection or screening.

Accordingly, the first identification step, as described, may be repeated one or more times, thereby enriching for the polynucleotides encoding the desired immunoglobulin subunit polypeptides. In order to repeat the first identification step, those polynucleotides, or pools of polynucleotides recovered as described above are introduced into a population of host cells capable of expressing bispecific antibodies encoded by the polynucleotides in the library. The host cells may be of the same type used in the first round of identification, or may be a different host cell, as long as they are capable of expressing bispecific antibodies. Polynucleotides encoding any additional libraries of immunoglobulin subunit polypeptides or polynucleotides encoding single fixed immunoglobulin subunit polypeptides are also introduced into these host cells as needed, and expression of bispecific antibodies, or antigen-binding fragments thereof is permitted. The cells or conditioned media are similarly contacted with antigen, and polynucleotides encoding the immunoglobulin subunit polypeptides being identified are again recovered from those cells or pools of host cells which express a bispecific antibody that specifically binds two non-identical epitopes of an antigen. These steps may be repeated one or more times, resulting in enrichment for polynucleotides derived from one or more libraries which encode various immunoglobulin subunit polypeptides which, as part of an bispecific antibody, or antigen-binding fragment thereof, specifically binds two non-identical epitopes of the antigen.

Following one or more rounds of enrichment, the diversity of the recovered subset of polynucleotides encoding the subunit polypeptide of the bispecific immunoglobulin being identified should approach or be equal to one, and resultant polynucleotides are then “isolated.”

Following suitable enrichment for the desired polynucleotides from the various libraries as described above, those polynucleotides which have been recovered are “isolated,” i.e., they are substantially removed from their native environment and are largely separated from polynucleotides in the library which do not encode desired immunoglobulin subunit polypeptides. For example, cloned polynucleotides contained in a vector are considered isolated for the purposes of the present invention. It is understood that two or more different immunoglobulin subunit polypeptides, e.g., two or more of the same immunoglobulin subunit polypeptide with different CDRs but binding to the same epitope, or a pair of immunoglobulin subunit polypeptides, e.g., a heavy chain and a light chain, which together comprise an antigen binding domain of interest, which specifically bind the same antigen can be recovered by the methods described herein. Accordingly, a mixture of polynucleotides which encode polypeptides binding to the same antigen is also considered to be “isolated.” Further examples of isolated polynucleotides include those maintained in heterologous host cells or purified (partially or substantially) DNA molecules in solution. However, a polynucleotide contained in a clone that is a member of a mixed library and that has not been isolated from other clones of the library, e.g., by virtue of encoding an antigen-specific immunoglobulin subunit polypeptide, is not “isolated” for the purposes of this invention. For example, a polynucleotide contained in a virus vector is “isolated” after it has been recovered, and plaque purified, and a polynucleotide contained in a plasmid vector is isolated after it has been expanded from a single bacterial colony.

Given that an antigen may comprise two or more epitopes, and several different immunoglobulin molecules may bind to any given epitope, it is contemplated that several suitable polynucleotides, e.g., two, three, four, five, ten, 100 or more polynucleotides, may be recovered from the first identification step of this embodiment, all of which may encode an immunoglobulin subunit polypeptide which, when combined with other suitable immunoglobulin subunit polypeptides, will form a bispecific antibody, or antigen binding fragment thereof, capable of specifically binding two non-identical epitopes of an antigen or antigens of interest. It is contemplated that each different polynucleotide recovered from any given library would be separately isolated. However, these polynucleotides may be isolated as a group of polynucleotides which encode polypeptides with the same antigen specificity, and these polynucleotides may be “isolated” together. Such mixtures of polynucleotides, whether separately isolated or collectively isolated, may be introduced into host cells in a second identification step, as explained below, either individually, or with two, three, four, five, ten, one hundred or more of the polynucleotides pooled together.

According to the sequential methods, once one or more suitable polynucleotides are recovered and isolated from the library or libraries screened in the first identification step, in the second or subsequent identification steps of this embodiment, one or more polynucleotides are identified in additional libraries which encode immunoglobulin subunit polypeptides which are capable of associating with the immunoglobulin subunit polypeptide(s) encoded by the polynucleotides isolated in the first identification step, and any fixed immunoglobulin subunit polypeptides, to form a bispecific antibody, or antigen-binding fragment thereof, which specifically binds at least two non-identical epitopes on an antigen or antigens of interest, or has a desired functional characteristic. Since a bispecific antibody of the present invention comprises at least two different heavy chains and/or at least two different light chains, it may be necessary to sequentially screen two, three, four, or more libraries of polynucleotides encoding immunoglobulin subunit polypeptides according to this method

Accordingly, the second identification step or subsequent identification steps comprise introducing into a population of host cells capable of expressing a bispecific antibody additional libraries of polynucleotides encoding immunoglobulin subunit polypeptides which combine with the immunoglobulin subunit polypeptides encoded by the polynucleotides isolated in the first identification step, introducing into the same population of host cells at least one of the polynucleotides isolated as described above, as well as any desired polynucleotides encoding fixed immunoglobulin subunit polypeptides of a known specificity, permitting expression of bispecific antibodies, or antigen-binding fragments thereof, contacting the expressed antibodies with the specific antigen or antigens of interest, and recovering polynucleotides of the second or subsequent libraries encoding immunoglobulin subunit polypeptides, which, as part of a bispecific antibody, bind at least two non-identical epitopes of the antigen(s) of interest. The second and subsequent identification steps are thus carried out very similarly to the first identification step, except that the immunoglobulin subunit polypeptides encoded by the polynucleotides of the second or subsequent libraries are combined in the host cells with those polynucleotides isolated in the first identification step, and any polynucleotides encoding fixed immunoglobulin subunit polypeptides of known specificity not contained in the isolated polynucleotides, and not contained in the library being screened.

As mentioned above, a single cloned polynucleotide isolated in the first identification step, or alternatively a pool of several polynucleotides isolated in the first identification step may be introduced simultaneously in the second or subsequent steps. As with the first identification step described above, one or more rounds of enrichment are carried out, i.e., either selection or screening of successively smaller pools of polynucleotides, thereby enriching for polynucleotides of the second or subsequent libraries which encode immunoglobulin subunit polypeptides which, as part of an bispecific antibody, or antigen-binding fragment thereof, specifically bind at least two non-identical epitopes of the antigen(s) of interest, or exhibits a desired functional characteristic. Also as with the first identification step, following one or rounds of enrichment, one or more desired polynucleotides from the additional libraries are then isolated. If a pool of isolated polynucleotides from the first identification step is used in the earlier rounds of enrichment during the second identification step, subsequent enrichment rounds may utilize smaller pools of polynucleotides isolated in the first identification step, or even individual cloned polynucleotides isolated in the first identification step. For any individual polynucleotide isolated in the first identification step which is then used in the second or subsequent identification steps for polynucleotides of the additional libraries, it is possible that several, i.e. two, three, four, five, ten, one hundred, or more polynucleotides may be isolated from the additional libraries which encode immunoglobulin subunit polypeptides capable of associating with an immunoglobulin subunit polypeptide encoded by a polynucleotide isolated in the first identification step, and any fixed immunoglobulin subunit polypeptides, to form a bispecific antibody, or antigen binding fragment thereof, which specifically binds two non-identical epitopes on the the antigen(s) of interest.

In certain identification schemes in which bispecific antibodies are expressed on the surface of host cells, the host cells themselves are “contacted” with antigen by a method which will allow an antigen, which specifically is recognized by a CDR of an immunoglobulin molecule expressed on the surface of the host cell, to bind to the CDR, thereby allowing the host cells which specifically bind the antigen to be distinguished from those host cells which do not bind the antigen. Any method which allows host cells expressing a bispecific antibody to interact with two non-identical epitopes of the antigen is included. For example, if the host cells are in suspension, and the antigen is attached to a solid substrate, cells which specifically bind to the antigen will be trapped on the solid substrate, allowing those cells which do not bind the antigen to be washed away, and the bound cells to be subsequently recovered. Of course, screening and/or selection schemes for host cells expressing bispecific immunoglobulin molecules are designed to detect binding of the antibody to two non-identical epitopes of the antigen. Alternatively, if the host cells are attached to a solid substrate, and by specifically binding at least two non-identical epitopes of the antigen cells are caused to be released from the substrate (e.g., by cell death), they can be recovered from the cell supernatant. Preferred methods by which to allow host cells of the invention to contact antigen, especially using libraries constructed in vaccinia virus vectors by trimolecular recombination, are disclosed herein.

In a preferred screening method for the detection of multispecific antibodies expressed on the surface of host cells, the host cells of the present invention are incubated with a selecting antigen that has been labeled directly with fluorescein-5-isothiocyanate (FITC) or indirectly with biotin then detected with FITC-labeled streptavidin. Other fluorescent probes can be employed which will be familiar to those practiced in the art. During the incubation period, the labeled selecting antigen binds the antigen-specific immunoglobulin molecules. Cells expressing an antibody receptor for a specific fluorescence tagged antigen can be selected by fluorescence activated cell sorting, thereby permitting the host cells which specifically bind the antigen to be distinguished from those host cells which do not bind the antigen. With the advent of cell sorters capable of sorting more than 1×10⁸ cells per hour, it is feasible to screen large numbers of cells infected with recombinant vaccinia libraries of immunoglobulin genes to select the subset of cells that express specific antibody receptors to the selecting antigen.

Vectors. In constructing bispecific antibody libraries in eukaryotic cells, whether for expression as secreted antibodies or on the surface of host cells, any standard vector which allows expression in eukaryotic cells may be used. For example, the library could be constructed in a virus, plasmid, phage, or phagemid vector as long as the particular vector chosen comprises transcription and translation regulatory regions capable of functioning in eukaryotic cells. In certain embodiments, antibody libraries as described above are constructed in eukaryotic virus vectors.

Eukaryotic virus vectors may be of any type, e.g., animal virus vectors or plant virus vectors. The naturally-occurring genome of the virus vector may be RNA, either positive strand, negative strand, or double stranded, or DNA, and the naturally-occurring genomes may be either circular or linear. Of the animal virus vectors, those that infect either invertebrates, e.g., insects, protozoans, or helminth parasites; or vertebrates, e.g., mammals, birds, fish, reptiles, and amphibians are included. The choice of virus vector is limited only by the maximum insert size, and the level of protein expression achieved. Suitable virus vectors are those that infect yeast and other fungal cells, insect cells, protozoan cells, plant cells, bird cells, fish cells, reptilian cells, amphibian cells, or mammalian cells, with mammalian virus vectors being particularly preferred. Any standard virus vector could be used in the present invention, including, but not limited to poxvirus vectors (e.g., vaccinia virus), herpesvirus vectors (e.g., herpes simplex virus), adenovirus vectors, baculovirus vectors, retrovirus vectors, picorna virus vectors (e.g., poliovirus), alphavirus vectors (e.g., sindbis virus), and enterovirus vectors (e.g., mengovirus). DNA virus vectors, e.g., poxvirus, herpes virus, baculovirus, and adenovirus used generally. As described in more detail below, the poxviruses, particularly orthopoxviruses, and especially vaccinia virus, are utilized in certain specific embodiments. In certain embodiments, host cells are utilized which are permissive for the production of infectious viral particles of whichever virus vector is chosen. Many standard virus vectors, such as vaccinia virus, have a very broad host range, thereby allowing the use of a large variety of host cells.

As mentioned supra, the various libraries of the invention may be constructed in the same vector, or may be constructed in different vectors. However, in preferred embodiments, the libraries are prepared such that polynucleotides of one of the libraries can be conveniently recovered, e.g., separated, from the polynucleotides of the other libraries in the first or subsequent identification steps. For example, in the first identification step, if the library being screened is constructed in a virus vector, and any additional libraries are constructed in a plasmid vector, the polynucleotides of the library being screened are easily recovered as infectious virus particles, while the polynucleotides of the other libraries are left behind with cellular debris. Similarly, in the second or subsequent identification steps, if the library being screened in that step is constructed in a virus vector, while the polynucleotides isolated in the first identification step are introduced in a plasmid vector, infectious virus particles containing polynucleotides of the library being screened are easily recovered.

When polynucleotides are introduced into host cells in a plasmid vector, the immunoglobulin subunit polypeptides encoded by polynucleotides comprised in such plasmid vectors may be operably associated with transcriptional regulatory regions which are driven by proteins encoded by virus vector which contains the library being screened. For example, if the library being screened is constructed in a poxvirus vector, and any additional libraries are constructed in a plasmid vectors, the polynucleotides encoding immunoglobulin subunit polypeptides constructed in the plasmid library may be operably associated with a transcriptional control region, preferably a promoter, which functions in the cytoplasm of poxvirus-infected cells. Similarly in the second identification step, if it is desired to insert the polynucleotides isolated in the first identificdation step into a plasmid vector, and the library being screened is constructed in a poxvirus vector, the polynucleotides isolated from the first library and inserted into plasmids may be operably associated with a transcriptional regulatory region, preferably a promoter, which functions in the cytoplasm of poxvirus-infected cells. Suitable examples of such transcriptional control regions are disclosed herein. In this way, the polynucleotides contained in plasmid vectors are only expressed in those cells which have also been infected by a poxvirus.

However, it is convenient to be able to maintain all libraries, as well as those polynucleotides isolated from the one or more libraries, in just a virus vector rather than having to maintain the libraries in two different vector systems. Accordingly, for the purpose of differential recovery of recombinants from one library relative to another, the present invention provides that samples of the libraries, maintained in a virus vector, are inactivated such that the virus vector infects cells and the genome of virus vector is transcribed and the proteins contained therein are expressed, but the vector is not replicated, i.e., when the virus vector is introduced into cells, gene products carried on the virus genome, e.g., immunoglobulin subunit polypeptides, are expressed, but infectious virus particles are not produced.

Inactivation of libraries constructed in eukaryotic virus vectors may be carried out by treating a sample of the library constructed in a virus vector with 4′-aminomethyl-trioxsalen (psoralen) and then exposing the virus vector to ultraviolet (UV) light. Psoralen and UV inactivation of viruses is well known to those of ordinary skill in the art. See, e.g., Tsung, K., et al., J. Virol. 70:165-171 (1996), which is incorporated herein by reference in its entirety.

Psoralen treatment typically comprises incubating a cell-free sample of the virus vector with a concentration of psoralen ranging from about 0.1 μg/ml to about 20 μg/ml, preferably about 1 μg/ml to about 17.5 μg/ml, about 2.5 μg/ml to about 15 μg/ml, about 5 μg/ml to about 12.5 μg/ml, about 7.5 μg/ml to about 12.5 μg/ml, or about 9 μg/ml to about 11 μg/ml. Accordingly, the concentration of psoralen may be about 0.1 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml. Typically, the concentration of psoralen is about 10 μg/ml. As used herein, the term “about” takes into account that measurements of time, chemical concentration, temperature, pH, and other factors typically measured in a laboratory or production facility are never exact, and may vary by a given amount based on the type of measurement and the instrumentation used to make the measurement.

The incubation with psoralen is typically carried out for a period of time prior to UV exposure. This time period preferably ranges from about one minute to about 20 minutes prior to the UV exposure. Preferably, the time period ranges from about 2 minutes to about 19 minutes, from about 3 minutes to about 18 minutes, from about 4 minutes to about 17 minutes, from about 5 minutes to about 16 minutes, from about 6 minutes to about 15 minutes, from about 7 minutes to about 14 minutes, from about 8 minutes to about 13 minutes, or from about 9 minutes to about 12 minutes. Accordingly, the incubation time may be about 1 minute, about 2 minutes, about three minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In certain embodiments, the incubation is carried out for 10 minutes prior to the UV exposure.

The psoralen-treated viruses are then exposed to UV light. The UV may be of any wavelength, but is preferably long-wave UV light, e.g., about 365 nm. Exposure to UV is carried out for a time period ranging from about 0.1 minute to about 20 minutes. For example, the time period ranges from about 0.2 minute to about 19 minutes, from about 0.3 minute to about 18 minutes, from about 0.4 minute to about 17 minutes, from about 0.5 minute to about 16 minutes, from about 0.6 minute to about 15 minutes, from about 0.7 minute to about 14 minutes, from about 0.8 minute to about 13 minutes, from about 0.9 minute to about 12 minutes from about 1 minute to about 11 minutes, from about 2 minutes to about 10 minutes, from about 2.5 minutes to about 9 minutes, from about 3 minutes to about 8 minutes, from about 4 minutes to about 7 minutes, or from about 4.5 minutes to about 6 minutes. Accordingly, the incubation time may be about 0.1 minute, about 0.5 minute, about 1 minute, about 2 minutes, about three minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In certain embodiments, the virus vector is exposed to UV light for a period of about 5 minutes.

The ability to assemble and express bispecific antibodies or antigen-binding fragments thereof in eukaryotic cells from two or more libraries of polynucleotides encoding immunoglobulin subunit polypeptides provides a significant improvement over the methods of producing single-chain antibodies in bacterial systems, in that the two-step selection process can be the basis for selection of bispecific antibodies or antigen-binding fragments thereof with a variety of specificities.

Examples of specific embodiments which further illustrate, but do not limit this embodiment, are provided in the Examples below. As described in detail, supra, identification of specific immunoglobulin subunit polypeptides, e.g., immunoglobulin heavy and light chains, is typcially accomplished in at least two phases, however, identification may be carried out in one step. First, two or more libraries of polynucleotides encoding diverse heavy chains from immunoglobulin producing cells of either naïve or immunized donors is constructed in a eukaryotic virus vector, for example, a poxvirus vector, and a similarly diverse library of polynucleotides encoding immunoglobulin light chains is constructed either in a plasmid vector, in which expression of the recombinant gene is regulated by a virus promoter, or in a eukaryotic virus vector which has been inactivated, e.g., through psoralen and UV treatment. Heavy chain libraries are constructed so as to promote formation of bivalent, or bispecific tetravalent antibodies, as described herein. Host cells capable of expressing immunoglobulin molecules, or antigen-binding fragments thereof, are infected with virus vector encoding the heavy chain library at a multiplicity of infection of about 1 (MOI=1). Two or more libraries of diverse heavy chains having complementary heterdimerization domains may be screened as well. In addition, complexity may be reduced by using one or more immunoglobulin subunit polypeptides, e.g., a heavy or light chain, with a known specificity. “Multiplicity of infection” refers to the average number of virus particles available to infect each host cell. For example, if an MOI of 1, i.e., an infection where, on average, each cell is infected by one virus particle, is desired, the number of infectious virus particles to be used in the infection is adjusted to be equal to the number of cells to be infected.

According to this strategy, host cells are either transfected with the light chain plasmid library, or infected with the inactivated light chain virus library under conditions which allow, on average, 1, 2, 3, 4, 5, 6, 7, 8, 9, 10 or more separate polynucleotides encoding light chain polypeptides to be taken up and expressed in each cell. Under these conditions, a single host cell can express multiple immunoglobulin molecules, or fragments thereof, with different light chains associated with the same heavy chains in characteristic H₂L₂ or H₄L₄ structures in each host cell.

It will be appreciated by those of ordinary skill in the art that controlling the number of plasmids taken up by a cell is difficult, because successful transfection depends on inducing a competent state in cells which may not be uniform and could lead to taking up variable amounts of DNA. Accordingly, in those embodiments where it is desired to carefully control the number of polynucleotides from the second library which are introduced into each infected host cell, the use of an inactivated virus vector is indicated, because the multiplicity of infection of viruses is more easily controlled.

The expression of multiple light chains in a single host cell, associated with a single heavy chain, has the effect of reducing the avidity of specific antigen immunoglobulin, but may be beneficial for selection of relatively high affinity binding sites. As used herein, the term “affinity” refers to a measure of the strength of the binding of an individual epitope with the CDR of an immunoglobulin molecule. See, e.g., Harlow at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. Where an increased avidity of antibody binding is desired, a preferred embodiment of the invention is to employ libraries of polynucleotides that encode bispecific tetravalent antibodies that may comprise multiple binding sites for a single epitope.

As will be appreciated by those of ordinary skill in the art, if a host cell expresses immunoglobulin molecules, each comprising a given heavy chain, but where different immunoglobulin molecules comprise different light chains, the “avidity” of those antibodies for a given antigen will be reduced. However, the possibility of recovering a group of immunoglobulin molecules which are related in that they comprise a common heavy chain, but which, through association with different light chains, react with a particular antigen with a spectrum of affinities, is increased. Accordingly, by adjusting the number of different light chains, or fragments thereof, which are allowed to associate with a certain number of heavy chains, or fragments thereof in a given host cell, the present invention provides a method to select for and enrich for immunoglobulin molecules, or antigen-binding fragments thereof, with varied affinity levels.

In utilizing this strategy in the first identification step of the method for selecting bispecific antibodies, or antigen-binding fragments thereof as described herein, the library being screened is typically constructed in a eukaryotic virus vector, and the host cells are infected with the library at an MOI ranging from about 1 to about 10, preferably about 1, 2, 3, or 4, while any additional libraries are introduced under conditions which allow up to 20 polynucleotides of those libraries to be taken up by each infected host cell. For example, if an additional library is constructed in an inactivated virus vector, the host cells are infected with that library at an MOI ranging from about 1 to about 20, although MOIs higher than this range may be desirable depending on the virus vector used and the characteristics of the immunoglobulin molecules desired. If the additional libraries are constructed in a plasmid vector, transfection conditions are adjusted to allow anywhere from 0 plasmids to about 20 plasmids to enter each host cell. Selection for lower or higher affinity responses to antigen is controlled by increasing or decreasing the average number of polynucleotides of the additional libraries allowed to enter each infected cell.

In certain embodiments described herein, one or more fixed polynucleotides encoding immunoglobulin subunit polypeptides which contribute to antigen binding domains recognizing an epitope of known specificity are utilized, in the first or subsequent identification steps. In this situation, the polynucleotides encoding fixed immunoglobulin subunit polypeptides are introduced into host cells under conditions which allow at least about 1 polynucleotide per host cell. Where a library of first heavy chain subunit polypeptides is being screened using a fixed second heavy chain subunit polypeptide, the ratio of fixed heavy chains to variable heavy chains may affect the formation of “productive” bispecific antibodies. Generally, where a library of polynucleotides encoding first heavy chain subunit polypeptides is introduced as an infectious virus vector at a certain MOI, non-infectious vectors carrying polynucleotides encoding the fixed second heavy chain subunit polypeptide are introduced to allow about the same number second heavy chain-encoding polynucleotides to enter a cell. In certain embodiments, it is desireable for the ratio of variable first heavy chain-encoding polynucleotides to fixed second heavy chain encoding polynucleotides to be about 1/4, 1/3, 1/2, 1, 2, 3, or 4. However, in those instances where the only heavy chain or light chain subunit polypeptide used is fixed, since all the polynucleotides encoding a given fixed immunoglobulin subunit polypeptide will be the same, i.e., copies of a cloned polynucleotide, the number of polynucleotides introduced into any given host cell is less important. For example, if a cloned polynucleotide encoding a fixed immunoglobulin subunit polypeptide is contained in an inactivated virus vector, that vector would be introduced at an MOI of about 1, but an MOI greater than 1 would be acceptable. Similarly, if a cloned polynucleotide encoding a fixed immunoglobulin subunit polypeptide is introduced in a plasmid vector, the number of plasmids which are introduced into any given host cell is of little importance, rather, transfection conditions should be adjusted to insure that at least one polynucleotide is introduced into each host cell.

An alternative embodiment may be utilized if, for example, several different polynucleotides encoding two or more fixed immunoglobulin subunit polypeptides are used. In this embodiment, pools of two or more different polynucleotides encoding fixed immunoglobulin subunit polypeptides may be advantageously introduced into host cells infected with the first or subsequent libraries of polynucleotides. In this situation, if the polynucleotides encoding fixed immunoglobulin subunit polypeptides are contained in an inactivated virus vector, an MOI of inactivated virus particles of greater than about 1, e.g., about 2, about 3, about 4, about 5, or more may be beneficial, or if the polynucleotides encoding fixed immunoglobulin subunit polypeptides are contained in a plasmid vector, conditions which allow at least about 2, 3, 4, 5, or more polynucleotides to enter each cell, may be used.

Where the library being screened is constructed in a virus vector, host cells are infected with that library at an MOI ranging from about 1-9, about 1-8, about 1-7, about 1-6, about 1-5, about 1-4, or about 1-2. In other words, host cells are infected with the library being screened at an MOI of about 10, about 9, about 8, about 7, about 6, about 5, about 4, about 3, about 2, or about 1. In certain embodiments, host cells are infected with the library being screened at an MOI of about 1.

Where the second and subsequent libraries are constructed in a plasmid vector, the plasmid vector is introduced into host cells under conditions which allow up to about 19, about 18, about 17, about 16, about 15, about 14, about 13, about 12, about 10, about 9, about 8, about 7, about 6, about 5, about 4, about 3 about 2, or about 1 polynucleotide(s) of the second library to be taken up by each infected host cell. Generally, where the second library is constructed in a plasmid vector, the plasmid vector is introduced into host cells under conditions which allow up to about 10 polynucleotides of the second library to be taken up by each infected host cell.

Similarly, where the second and subsequent libraries are constructed in an inactivated virus vector, it is introduced into host cells at an MOI ranging from about 1-19, about 2-18, about 3-17, about 4-16, about 5-15, about 6-14, about 7-13, about 8-12, or about 9-11. In other words, host cells are infected with the second and subsequent libraries at an MOI of about 20, about 19, about 18, about 17, about 16, about 15, about 14, about 13, about 12, about 11, about 10, about 9, about 8, about 7, about 6, about 5, about 4, about 3, about 2, or about 1. For example, host cells are infected with the second and subsequent libraries at an MOI of about 10. As will be understood by those of ordinary skill in the art, the titer, and thus the “MOI” of an inactivated virus cannot be directly measured, however, the titer may be inferred from the titer of the starting infectious virus stock which was subsequently inactivated.

In one embodiment, the first library is constructed in a virus vector and the second library is constructed in a virus vector which has been inactivated, the host cells are infected with said first library at an MOI of about 1, and the host cells are infected with the second library at an MOI of about 10 or less.

In the present invention, an exemplary virus vector is derived from a poxvirus, e.g., vaccinia virus. If a first library encoding a plurality of immunoglobulin subunit polypeptides is constructed in a poxvirus vector and the expression of additional immunoglobulin subunit polypeptides, encoded by the second or subsequent libraries constructed either in a plasmid vector or an inactivated virus vector, are regulated by a poxvirus promoter, high levels of the immunoglobulin subunit polypeptides encoded by the second or subsequent libraries are expressed in the cytoplasm of the poxvirus infected cells without a requirement for nuclear integration.

In the second or subsequent identification step of the immunoglobulin identification as described above, the second or subsequent libraries are conveniently constructed in an infectious eukaryotic virus vector, and the host cells are infected with the second library at an MOI ranging from about 1 to about 10. For example, where the second or subsequent libraries are constructed in a virus vector, host cells are infected with the second library at an MOI ranging from about 1-9, about 1-8, about 1-7, about 1-6, about 1-5, about 1-4, or about 1-2. In other words, host cells are infected with the second or subsequent libraries at an MOI of about 10, about 9, about 8, about 7, about 6, about 5, about 4, about 3, about 2, or about 1. Typically, host cells are infected with the second or subsequent libraries at an MOI of about 1.

In the second identification step, polynucleotides identified in the first identification step, e.g., polynucleotides from the first library and any subsequent libraries which were screened at the same time, have been isolated. In certain embodiments, a single first library polynucleotide or a small number of first library polynucleotides, i.e., clones, are introduced into the host cells used to identify polynucleotides from the second or subsequent libraries. In this situation, the polynucleotides isolated from the first library are introduced into host cells under conditions which allow at least about 1 of each different polynucleotide per host cell. However, since all the polynucleotides being introduced from the first library will be either the same or comprise a small number of different polynucleotides, i.e., copies of cloned polynucleotides, the number of polynucleotides introduced into any given host cell is less important. For example, if a cloned polynucleotide isolated from the first library is contained in an inactivated virus vector, that vector would be introduced at an MOI of about 1, but an MOI greater than 1 would be acceptable. Similarly, if a cloned polynucleotide isolated from the first library is introduced in a plasmid vector, the number of plasmids which are introduced into any given host cell is of little importance, rather, transfection conditions should be adjusted to insure that at least one polynucleotide is introduced into each host cell. An alternative embodiment may be utilized if, for example, several different polynucleotides were isolated from the first library. In this embodiment, pools of two or more different polynucleotides isolated from the first library may be advantageously introduced into host cells infected with the second library of polynucleotides. In this situation, if the polynucleotides isolated from the first library are contained in an inactivated virus vector, an MOI of inactivated virus particles of greater than about 1, e.g., about 2, about 3, about 4, about 5, or more may be utilized, or if the polynucleotides isolated from the first library are contained in a plasmid vector, conditions which allow at least about 2, 3, 4, 5, or more polynucleotides to enter each cell, may be utilized. Similarly in a third or subsequent identification step, polynucleotides identified in the first identification step and any polynucleotides identified in the second identification step, have been isolated, and the same guidelines as described above are followed.

Poxvirus Vectors. As noted above, a preferred virus vector for use in the present invention is a poxvirus vector. “Poxvirus” includes any member of the family Poxyiridae, including the subfamililes Chordopoxyiridae (vertebrate poxviruses) and Entomopoxyiridae (insect poxviruses). See, for example, B. Moss in: Virology, 2d Edition, B. N. Fields, D. M. Knipe et al., Eds., Raven Press, p. 2080 (1990). The chordopoxviruses comprise, inter alia, the following genera: Orthopoxvirus (e.g., vaccinia, variola virus, raccoon poxvirus); Avipoxvirus (e.g., fowlpox); Capripoxvirus (e.g, sheeppox) Leporipoxvirus (e.g., rabbit (Shope) fibroma, and myxoma); and Suipoxvirus (e.g., swinepox). The entomopoxviruses comprise three genera: A, B and C. In the present invention, orthopoxviruses are preferred. Vaccinia virus is the prototype orthopoxvirus, and has been developed and is well-characterized as a vector for the expression of heterologous proteins. In the present invention, vaccinia virus vectors, particularly those that have been developed to perform trimolecular recombination, are preferred. However, other orthopoxviruses, in particular, raccoon poxvirus have also been developed as vectors and in some applications, have superior qualities.

Poxviruses are distinguished by their large size and complexity, and contain similarly large and complex genomes. Notably, poxvirus replication takes place entirely within the cytoplasm of a host cell. The central portions of poxvirus genomes are similar, while the terminal portions of the virus genomes are characterized by more variability. Accordingly, it is thought that the central portion of poxvirus genomes carry genes responsible for essential functions common to all poxviruses, such as replication. By contrast, the terminal portions of poxvirus genomes appear responsible for characteristics such as pathogenicity and host range, which vary among the different poxviruses, and may be more likely to be non-essential for virus replication in tissue culture. It follows that if a poxvirus genome is to be modified by the rearrangement or removal of DNA fragments or the introduction of exogenous DNA fragments, the portion of the naturally-occurring DNA which is rearranged, removed, or disrupted by the introduction of exogenous DNA is preferably in the more distal regions thought to be non-essential for replication of the virus and production if infectious virions in tissue culture.

The naturally-occurring vaccinia virus genome is a cross-linked, double stranded linear DNA molecule, of about 186,000 base pairs (bp), which is characterized by inverted terminal repeats. The genome of vaccinia virus has been completely sequenced, but the functions of most gene products remain unknown. Goebel, S. J., et al., Virology 179:247-266, 517-563 (1990); Johnson, G. P., et al., Virology 196:381-401. A variety of non-essential regions have been identified in the vaccinia virus genome. See, e.g., Perkus, M. E., et al., Virology 152:285-97 (1986); and Kotwal, G. J. and Moss B., Virology 167:524-37.

Specific examples of additional non-essential regions in the vaccinia virus genome for insertion of foreign polynucleotides include, but are not limited to: the vaccinia virus F7L open reading frame in the Hind III F fragment (Coupar, B E et al., J Gen Virol. 81:431-439 (2000)); the vaccinia virus I4L locus (ribonucleotide reductase-encoding gene) (Howley, P M et al. Gene 172:223-227 (1996)); the vaccinia virus D6, D7, D8, D9, D10, D11, D12, D13 and A1 genes (Binns, M M et al, J Gen Virol 71:2873-2881 (1990)); a 14.5-kbp region located at the left end of the standard vaccinia virus genome, extending from within the inverted terminal repetition (ITR) of the HindIII C fragment to the end of the HindIII N fragment containing 17 contiguous open reading frames (ORFs) (Kotwal, G J, and Moss, B Virology 167:534-527 (1988)); the vaccinia virus SalF5R gene (Duncan, S A and Smith G L J Gen Virol. 73:1235-1242 (1992)); the vaccinia virus hemagglutinin gene (Shida, H Virology 150:451-462 (1986)); and the vaccinia virus K1L locus (Wild, T F, et al., J Gen Virol. 73:359-367 (1992)). As will be understood by one of ordinary skill in the art, many, if not most of these nonessential regions have homologs in related poxviruses.

In those embodiments where poxvirus vectors, in particular vaccinia virus vectors, are used to express immunglobulin subunit polypeptides, any suitable poxvirus vector may be used. It is preferred that the libraries of immunoglobulin subunit polypeptides be carried in a region of the vector which is non-essential for growth and replication of the vector so that infectious viruses are produced. Although a variety of non-essential regions of the vaccinia virus genome have been characterized as listed above, the most widely used locus for insertion of foreign genes is the thymidine kinase locus, located in the HindIII J fragment in the genome. In certain vaccinia virus vectors, the tk locus has been engineered to contain one or two unique restriction enzyme sites, allowing for convenient use of the trimolecular recombination method of library generation. See infra, and also Zauderer, PCT Publication No. WO 00/028016.

Libraries of polynucleotides encoding immunoglobulin subunit polypeptides are inserted into poxvirus vectors, particularly vaccinia virus vectors, under operable association with a transcriptional control region which functions in the cytoplasm of a poxvirus-infected cell.

Poxvirus transcriptional control regions comprise a promoter and a transcription termination signal. Gene expression in poxviruses is temporally regulated, and promoters for early, intermediate, and late genes possess varying structures. Certain poxvirus genes are expressed constitutively, and promoters for these “early-late” genes bear hybrid structures. Synthetic early-late promoters have also been developed. See Hammond J. M., et al., J. Virol. Methods 66:135-8 (1997); Chakrabarti S., et al., Biotechniques 23:1094-7 (1997). In the present invention, any poxvirus promoter may be used, but use of early, late, or constitutive promoters may be desirable based on the host cell and/or selection scheme chosen. Typically, the use of constitutive promoters is preferred.

Examples of early promoters include the 7.5-kD promoter (also a late promoter), the DNA pol promoter, the tk promoter, the RNA pol promoter, the 19-kD promoter, the 22-kD promoter, the 42-kD promoter, the 37-kD promoter, the 87-kD promoter, the H3′ promoter, the H6 promoter, the D1 promoter, the D4 promoter, the D5 promoter, the D9 promoter, the D12 promoter, the 13 promoter, the M1 promoter, and the N2 promoter. See, e.g., Moss, B., “Poxyiridae and their Replication” IN Virology, 2d Edition, B. N. Fields, D. M. Knipe et al., Eds., Raven Press, p. 2088 (1990). Early genes transcribed in vaccinia virus and other poxviruses recognize the transcription termination signal TTTTTNT, where N can be any nucleotide. Transcription normally terminates approximately 50 bp upstream of this signal. Accordingly, if heterologous genes are to be expressed from poxvirus early promoters, care must be taken to eliminate occurrences of this signal in the coding regions for those genes. See, e.g., Earl, P. L., et al., J. Virol. 64:2448-51 (1990).

Examples of late promoters include the 7.5-kD promoter, the MIL promoter, the 37-kD promoter, the 11-kD promotor, the 11L promoter, the 12L promoter, the 13L promoter, the 15L promoter, the 17L promoter, the 28-kD promoter, the H1L promoter, the H3L promoter, the H5L promoter, the H6L promoter, the H8L promoter, the D11L promoter, the D12L promotor, the D13L promoter, the A1L promoter, the A2L promoter, the A3L promoter, and the P4b promoter. See, e.g., Moss, B., “Poxyiridae and their Replication” IN Virology, 2d Edition, B. N. Fields, D. M. Knipe et al., Eds., Raven Press, p. 2090 (1990). The late promoters apparently do not recognize the transcription termination signal recognized by early promoters.

Constitutive promoters for use in the present invention include the synthetic early-late promoters described by Hammond and Chakrabarti, the H-5 early-late promoter, and the 7.5-kD or “p7.5” promoter. Examples utilizing these promoters are disclosed herein.

Antibody secretion by host cells infected with vaccinia virus is limited by the cytopathic effect (CPE) caused by virus infection. In addition, as will be discussed in more detail below, certain selection and screening methods based on host cell death require that the mechanisms leading to cell death occur prior to any cytopathic effect (CPE) caused by virus infection. The kinetics of the onset of CPE in virus-infected cells is dependent on the virus used, the multiplicity of infection, and the type of host cell. For example, in many tissue culture lines infected with vaccinia virus at an MOI of about 1, CPE is not significant until well after 48 to 72 hours post-infection. This allows a 2 to 3 day time frame for high level expression of immunoglobulin molecules, and antigen-based selection independent of CPE caused by the vector. However, this time frame may not be sufficient for certain selection methods, especially where higher MOIs are used, and further, the time before the onset of CPE may be shorter in a desired cell line. There is, therefore, a need for virus vectors, particularly poxvirus vectors such as vaccinia virus, with attenuated cytopathic effects so that, wherever necessary, the time frame of selection can be extended.

For example, certain attenuations are achieved through genetic mutation. These may be fully defective mutants, i.e., the production of infectious virus particles requires helper virus, or they may be conditional mutants, e.g., temperature sensitive mutants. Conditional mutants are particularly preferred, in that the virus-infected host cells can be maintained in a non-permissive environment, e.g., at a non-permissive temperature, during the period where host gene expression is required, and then shifted to a permissive environment, e.g., a permissive temperature, to allow virus particles to be produced. Alternatively, a fully infectious virus may be “attenuated” by chemical inhibitors which reversibly block virus replication at defined points in the infection cycle. Chemical inhibitors include, but are not limited to hydroxyurea and 5-fluorodeoxyuridine. Virus-infected host cells are maintained in the chemical inhibitor during the period where host gene expression is required, and then the chemical inhibitor is removed to allow virus particles to be produced.

A number of attenuated poxviruses, in particular vaccinia viruses, have been developed. For example, modified vaccinia Ankara (MVA) is a highly attenuated strain of vaccinia virus that was derived during over 570 passages in primary chick embryo fibroblasts (Mayr, A. et al., Infection 3:6-14 (1975)). The recovered virus deleted approximately 15% of the wild type vaccinia DNA which profoundly affects the host range restriction of the virus. MVA cannot replicate or replicates very inefficiently in most mammalian cell lines. A unique feature of the host range restriction is that the block in non-permissive cells occurs at a relatively late stage of the replication cycle. Expression of viral late genes is relatively unimpaired but virion morphogenesis is interrupted (Suter, G. and Moss, B., Proc Natl Acad Sci USA 89:10847-51 (1992); Carroll, M. W. and Moss, B., Virology 238:198-211 (1997)). The high levels of viral protein synthesis even in non-permissive host cells make MVA an especially safe and efficient expression vector. However, because MVA cannot complete the infectious cycle in most mammalian cells, in order to recover infectious virus for multiple cycles of selection it will be necessary to complement the MVA deficiency by coinfection or superinfection with a helper virus that is itself deficient and that can be subsequently separated from infectious MVA recombinants by differential expansion at low MOI in MVA permissive host cells.

Poxvirus infection can have a dramatic inhibitory effect on host cell protein and RNA synthesis. These effects on host gene expression could, under some conditions, interfere with the selection of specific poxvirus recombinants that have a defined physiological effect on the host cell. Some strains of vaccinia virus that are deficient in an essential early gene have been shown to have greatly reduced inhibitory effects on host cell protein synthesis. Attenuated poxviruses which lack defined essential early genes have also been described. See, e.g., U.S. Pat. Nos. 5,766,882, and 5,770,212, by Falkner, et al. Examples of essential early genes which may be rendered defective include, but are not limited to the vaccinia virus 17L, F18R, D13L, D6R, A8L, J1R, E7L, F11L, E4L, I1L, J3R, J4R, H7R, and A6R genes. A preferred essential early gene to render defective is the D4R gene, which encodes a uracil DNA glycosylase enzyme. Vaccinia viruses defective in defined essential genes are easily propagated in complementing cell lines which provides the essential gene product.

As used herein, the term “complementation” refers to a restoration of a lost function in trans by another source, such as a host cell, a host cell transfected with a gene mutated in the virus, or helper virus. The loss of function is caused by loss by the defective virus of the gene product responsible for the function. Thus, a defective poxvirus is a non-viable form of a parental poxvirus, and is a form that can become viable in the presence of complementation. The host cell, transfected host cell, or helper virus contains the sequence encoding the lost gene product, or “complementation element.” The complementation element should be expressible and stably integrated in the host cell, transfected host cell or helper virus, and preferably would be subject to little or no risk for recombination with the genome of the defective poxvirus.

Viruses produced in the complementing cell line are capable of infecting non-complementing cells, and further are capable of high-level expression of early gene products. However, in the absence of the essential gene product, host shut-off, DNA replication, packaging, and production of infectious virus particles does not take place.

In certain embodiments described herein, selection of desired target gene products expressed in a complex library constructed in vaccinia virus is accomplished through coupling induction of expression of the complementation element to expression of the desired target gene product. Since the complementation element is only expressed in those host cells expressing the desired gene product, only those host cells will produce infectious virus which is easily recovered.

The embodiments relating to vaccinia virus may be modified in ways apparent to one of ordinary skill in the art for use with any poxvirus vector. In the direct selection method, vectors other than poxvirus or vaccinia virus may be used.

The Tri-Molecular Recombination Method. Traditionally, poxvirus vectors such as vaccinia virus have not been used to identify previously unknown genes of interest from a complex libraries because a high efficiency, high titer-producing method of constructing and screening libraries did not exist for vaccinia. The standard methods of heterologous protein expression in vaccinia virus involve in vivo homologous recombination and in vitro direct ligation. Using homologous recombination, the efficiency of recombinant virus production is in the range of approximately 0.1% or less. Although efficiency of recombinant virus production using direct ligation is higher, the resulting titer is relatively low. Thus, the use of vaccinia virus vector has been limited to the cloning of previously isolated DNA for the purposes of protein expression and vaccine development.

Tri-molecular recombination, as disclosed in Zauderer, PCT Publication No. WO 00/028016, is a novel, high efficiency, high titer-producing method for cloning in vaccinia virus. Using the tri-molecular recombination method, the present inventors have achieved generation of recombinant viruses at efficiencies of at least 90%, and titers at least at least 2 orders of magnitude higher than those obtained by direct ligation.

Thus, in this embodiment, libraries of polynucleotides capable of expressing immunoglobulin subunit polypeptides are constructed in poxvirus vectors, preferably vaccinia virus vectors, by tri-molecular recombination.

By “tri-molecular recombination” or a “tri-molecular recombination method” is meant a method of producing a virus genome, e.g., a poxvirus genome, e.g., a vaccinia virus genome comprising a heterologous insert DNA, by introducing two nonhomologous fragments of a virus genome and a transfer vector or transfer DNA containing insert DNA into a recipient cell, and allowing the three DNA molecules to recombine in vivo. As a result of the recombination, a viable virus genome molecule is produced which comprises each of the two genome fragments and the insert DNA. Thus, the tri-molecular recombination method as applied to the present invention comprises: (a) cleaving an isolated virus genome, preferably a DNA virus genome, e.g., a linear DNA virus genome, and such as poxvirus or vaccinia virus genome, to produce a first viral fragment and a second viral fragment, where the first viral fragment is nonhomologous with the second viral fragment; (b) providing a population of transfer plasmids comprising polynucleotides which encode immunoglobulin subunit polypeptides, e.g., immunoglobulin light chains, first or second immunoglobulin heavy chains, or antigen-binding fragments of either, through operable association with a transcription control region, flanked by a 5′ flanking region and a 3′ flanking region, wherein the 5′ flanking region is homologous to said the viral fragment described in (a), and the 3′ flanking region is homologous to said second viral fragment described in (a); and where the transfer plasmids are capable of homologous recombination with the first and second viral fragments such that a viable virus genome is formed; (c) introducing the transfer plasmids described in (b) and the first and second viral fragments described in (a) into a host cell under conditions where a transfer plasmid and the two viral fragments undergo in vivo homologous recombination, i.e., trimolecular recombination, thereby producing a viable modified virus genome comprising a polynucleotide which encodes an immunoglobulin subunit polypeptide; and (d) recovering modified virus genomes produced by this technique. Preferably, the recovered modified virus genome is packaged in an infectious viral particle.

By “recombination efficiency” or “efficiency of recombinant virus production” is meant the ratio of recombinant virus to total virus produced during the generation of virus libraries of the present invention. As shown in Example 5, the efficiency may be calculated by dividing the titer of recombinant virus by the titer of total virus and multiplying by 100%. For example, the titer is determined by plaque assay of crude virus stock on appropriate cells either with selection (e.g., for recombinant virus) or without selection (e.g., for recombinant virus plus wild type virus). Methods of selection, particularly if heterologous polynucleotides are inserted into the viral thymidine kinase (tk) locus, are well-known in the art and include resistance to bromdeoxyuridine (BDUR) or other nucleotide analogs due to disruption of the tk gene. Examples of selection methods are described herein.

By “high efficiency recombination” is meant a recombination efficiency of at least about 1%, about 2%, about 2.5%, about 3%, about 3.5%, about 4%, about 5%, about 10%, about 20%, about 30%, about 40%, about 50%, about 60%, about 70%, about 75%, about 80%, about 85%, about 90%, about 95%, or about 99%.

A number of selection systems may be used, including but not limited to the thymidine kinase such as herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes which can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Proc. Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Together, the first and second viral fragments or “arms” of the virus genome, as described above, preferably contain all the genes necessary for viral replication and for production of infectious viral particles. Examples of suitable arms and methods for their production using vaccinia virus vectors are disclosed herein. See also Falkner et al., U.S. Pat. No. 5,770,212 for guidance concerning essential regions for vaccinia replication.

However, naked poxvirus genomic DNAs such as vaccinia virus genomes cannot produce infectious progeny without virus-encoded protein protein(s)/function(s) associated with the incoming viral particle. The required virus-encoded functions, include an RNA polymerase that recognizes the transfected vaccinia DNA as a template, initiates transcription and, ultimately, replication of the transfected DNA. See Dorner, et al. U.S. Pat. No. 5,445,953.

Thus, to produce infectious progeny virus by trimolecular recombination using a poxvirus such as vaccinia virus, the recipient cell preferably contains packaging function. The packaging function may be provided by helper virus, i.e., a virus that, together with the transfected naked genomic DNA, provides appropriate proteins and factors necessary for replication and assembly of progeny virus.

The helper virus may be a closely related virus, for instance, a poxvirus of the same poxvirus subfamily as vaccinia, whether from the same or a different genus. In such a case it is advantageous to select a helper virus which provides an RNA polymerase that recognizes the transfected DNA as a template and thereby serves to initiate transcription and, ultimately, replication of the transfected DNA. If a closely related virus is used as a helper virus, it is advantageous that it be attenuated such that formation of infectious virus will be impaired. For example, a temperature sensitive helper virus may be used at the non-permissive temperature. Preferably, a heterologous helper virus is used. Examples include, but are not limited to an avipox virus such as fowlpox virus, or an ectromelia virus (mouse pox) virus. In particular, avipoxviruses are preferred, in that they provide the necessary helper functions, but do not replicate, or produce infectious virions in mammalian cells (Scheiflinger, et al., Proc. Natl. Acad. Sci. USA 89:9977-9981 (1992)). Use of heterologous viruses minimizes recombination events between the helper virus genome and the transfected genome which take place when homologous sequences of closely related viruses are present in one cell. See Fenner & Comben, Virology 5:530 (1958); Fenner, Virology 8:499 (1959).

Alternatively, the necessary helper functions in the recipient cell is supplied by a genetic element other than a helper virus. For example, a host cell can be transformed to produce the helper functions constitutively, or the host cell can be transiently transfected with a plasmid expressing the helper functions, infected with a retrovirus expressing the helper functions, or provided with any other expression vector suitable for expressing the required helper virus function. See Dorner, et al. U.S. Pat. No. 5,445,953.

According to the trimolecular recombination method, the first and second viral genomic fragments are unable to ligate or recombine with each other in vivo, i.e., they do not contain compatible cohesive ends or homologous regions. In a preferred embodiment, a virus genome comprises a first recognition site for a first restriction endonuclease and a second recognition site for a second restriction endonuclease, and the first and second viral fragments are produced by digesting the viral genome with the appropriate restriction endonucleases to produce the viral “arms,” and the first and second viral fragments are isolated by standard methods. Ideally, the first and second restriction endonuclease recognition sites are unique in the viral genome, or alternatively, cleavage with the two restriction endonucleases results in viral “arms” which include the genes for all essential functions, i.e., where the first and second recognition sites are physically arranged in the viral genome such that the region extending between the first and second viral fragments is not essential for virus infectivity.

Where a vaccinia virus vector is used in the trimolecular recombination method, a vaccinia virus vector comprising a virus genome with two unique restriction sites within the tk gene is used. In certain vaccinia virus genomes, the first restriction enzyme is NotI, having the recognition site GCGGCCGC in the tk gene, and the second restriction enzyme is ApaI, having the recognition site GGGCCC in the tk gene. Examples are vaccinia virus vectors comprising a vH5/tk virus genome, a v7.5/tk virus genome or a vEL/tk virus genome.

According to this embodiment, a transfer plasmid with flanking regions capable of homologous recombination with the region of the vaccinia virus genome containing the thymidine kinase gene is used. A fragment of the vaccinia virus genome comprising the HindIII-J fragment, which contains the tk gene, is conveniently used.

Where the virus vector is a poxvirus, the insert polynucleotides are operably associated with poxvirus expression control sequences, for example, strong constitutive poxvirus promoters such as pH5, p7.5, or a synthetic early/late promoter.

Accordingly, a transfer plasmid of the present invention comprises a polynucleotide encoding an immunoglobulin subunit polypeptide, e.g., an heavy chain, and immunoglobulin light chain, or an antigen-binding fragment of a heavy chain or a light chain, through operable association with a vaccinia virus pH5 promoter, a p7.5 promoter, or a synthetic early/late promoter.

A preferred transfer plasmid of the present invention which comprises a polynucleotide encoding an immunoglobulin heavy chain polypeptide through operable association with a vaccinia virus p7.5 promoter is pVHE, which comprises the sequence designated herein as SEQ ID NO:11. PCR-amplified heavy chain variable regions may be inserted in-frame into unique BssHII (at nucleotides 96-101 of SEQ ID NO:11), and BstEII (nucleotides 106-112 of SEQ ID NO:11) sites.

Furthermore, pVHE may be used in those embodiments where it is desired to transfer polynucleotides isolated from the first library into a plasmid vector for subsequent selection of polynucleotides of the second library as described above. Another transfer plasmid of the present invention which comprises a polynucleotide encoding an immunoglobulin kappa light chain polypeptide through operable association with a vaccinia virus p7.5 promoter is pVKE, which comprises the sequence designated herein as SEQ ID NO:12. PCR-amplified kappa light chain variable regions may be inserted in-frame into unique ApaLI (nucleotides 95-100 of SEQ ID NO:12), and XhoI (nucleotides 105-110 of SEQ ID NO:12) sites.

Furthermore, pVKE may be used in those embodiments where it is desired to have polynucleotides of the second library in a plasmid vector during the selection of polynucleotides of the first library as described above.

Another transfer plasmid of the present invention which comprises a polynucleotide encoding an immunoglobulin kappa light chain constant region polypeptide through operable association with a vaccinia virus p7.5 promoter is pVLE, which is designed to accept a lambda light chain variable region upstream of the kappa constant region. pVLE comprises the sequence designated herein as SEQ ID NO:13. PCR-amplified lambda light chain variable regions may be inserted in-frame into unique ApaLI (nucleotides 95-100 of SEQ ID NO:13) and HindIII (nucleotides 111-116 of SEQ ID NO:13) sites.

Furthermore, pVLE may be used in those embodiments where it is desired to have polynucleotides of the second library in a plasmid vector during the selection of polynucleotides of the first library as described above.

By “insert DNA” is meant one or more heterologous DNA segments to be expressed in the recombinant virus vector. According to the present invention, “insert DNAs” are polynucleotides which encode immunoglobulin subunit polypeptides. A DNA segment may be naturally occurring, non naturally occurring, synthetic, or a combination thereof. Methods of producing insert DNAs of the present invention are disclosed herein.

By “transfer plasmid” is meant a plasmid vector containing an insert DNA positioned between a 5′ flanking region and a 3′ flanking region as described above. The 5′ flanking region shares homology with the first viral fragment, and the 3′ flanking region shares homology with the second viral fragment. Preferably, the transfer plasmid contains a suitable promoter, such as a strong, constitutive vaccinia promoter where the virus vector is a poxvirus, upstream of the insert DNA. The term “vector” means a polynucleotide construct containing a heterologous polynucleotide segment, which is capable of effecting transfer of that polynucleotide segment into a suitable host cell. The polynucleotide contained in the vector is operably linked to a suitable control sequence capable of effecting expression of the polynucleotide in a suitable host. Such control sequences include a promoter to effect transcription, an optional operator sequence to control such transcription, a sequence encoding suitable mRNA ribosome binding sites, and sequences which control the termination of transcription and translation. As used herein, a vector may be a plasmid, a phage particle, a virus, a messenger RNA, or simply a potential genomic insert. Once transformed into a suitable host, the vector may replicate and function independently of the host genome, or may in some instances, integrate into the genome itself. Typical plasmid expression vectors for mammalian cell culture expression, for example, are based on pRK5 (EP 307,247), pSV16B (WO 91/08291) and pVL1392 (Pharmingen).

However, “a transfer plasmid,” as used herein, is not limited to a specific plasmid or vector. Any DNA segment in circular or linear or other suitable form may act as a vehicle for transferring the DNA insert into a host cell along with the first and second viral “arms” in the tri-molecular recombination method. Other suitable vectors include lambda phage, mRNA, DNA fragments, etc., as described herein or otherwise known in the art. A plurality of plasmids may be a “primary library” such as those described herein for lambda.

Modifications of Trimolecular Recombination. Trimolecular recombination can be used to construct cDNA libraries in vaccinia virus with titers of the order of about 10⁷ pfu. There are several factors that limit the complexity of these cDNA libraries or other libraries. These include: the size of the primary cDNA library or other library, such as a library of polynucleotides encoding immunoglobulin subunit polypeptides, that can be constructed in a plasmid vector, and the labor involved in the purification of large quantities (hundreds of micrograms) of virus “arms,” preferably vaccinia virus “arms” or other poxvirus “arms.” Modifications of trimolecular recombination that would allow for vaccinia or other virus DNA recombination with primary cDNA libraries or other libraries, such as polynucleotides encoding immunoglobulin subunit polypeptides, constructed in bacteriophage lambda or DNA or phagemids derived therefrom, or that would allow separate virus DNA arms to be generated in vivo following infection with a modified viral vector could greatly increase the quality and titer of the eukaryotic virus cDNA libraries or other libraries that are constructed using these methods.

Transfer of cDNA inserts from a Bacteriophage Lambda Library to Vaccinia Virus. Lambda phage vectors have several advantages over plasmid vectors for construction of cDNA libraries or other libraries, such as polynucleotides encoding immunoglobulin subunit polypeptides. Plasmid cDNA (or other DNA insert) libraries or linear DNA libraries are introduced into bacteria cells by chemical/heat shock transformation, or by electroporation. Bacteria cells are preferentially transformed by smaller plasmids, resulting in a potential loss of representation of longer cDNAs or other insert DNA, such as polynucleotides encoding immunoglobulin subunit polypeptides, in a library. In addition, transformation is a relatively inefficient process for introducing foreign DNA or other DNA into a cell requiring the use of expensive commercially prepared competent bacteria in order to construct a cDNA library or other library, such as polynucleotides encoding immunoglobulin subunit polypeptides. In contrast, lambda phage vectors can tolerate cDNA inserts of 12 kilobases or more without any size bias. Lambda vectors are packaged into virions in vitro using high efficiency commercially available packaging extracts so that the recombinant lambda genomes can be introduced into bacterial cells by infection. This results in primary libraries with higher titers and better representation of large cDNAs or other insert DNA, such as polynucleotides encoding immunoglobulin subunit polypeptides, than is commonly obtained in plasmid libraries.

To enable transfer of cDNA inserts or other insert DNA, such as polynucleotides encoding immunoglobulin subunit polypeptides, from a library constructed in a lambda vector to a eukaryotic virus vector such as vaccinia virus, the lambda vector must be modified to include vaccinia virus DNA sequences that allow for homologous recombination with the vaccinia virus DNA. The following example uses vaccinia virus homologous sequences, but other viruses may be similarly used. For example, the vaccinia virus HindIII J fragment (comprising the vaccinia tk gene) contained in plasmid p7.5/ATG0/tk (as described in Example 5, infra) can be excised using HindIII and SnaBI (3 kb of vaccinia DNA sequence), and subcloned into the HindIII/SnaBI sites of pT7Blue3 (Novagen cat no. 70025-3) creating pT7B3.Vtk. The vaccinia tk gene can be excised from this vector with SacI and SnaBI and inserted into the SacI/SmaI sites of Lambda Zap Express (Stratagene) to create lambda.Vtk. The lambda.Vtk vector will contain unique NotI, BamHI, SmaI, and SalI sites for insertion of cDNA downstream of the vaccinia 7.5k promoter. cDNA libraries can be constructed in lambda.Vtk employing methods that are well known in the art.

DNA from a cDNA library or other library, such as polynucleotides encoding immunoglobulin subunit polypeptides, constructed in lambda.Vtk, or any similar bacteriophage that includes cDNA inserts or other insert DNA with flanking vaccinia DNA sequences to promote homologous recombination, can be employed to generate cDNA or other insert DNA recombinant vaccinia virus. Methods are well known in the art for excising a plasmid from the lambda genome by coinfection with a helper phage (ExAssist phage, Stratagene cat no. 211203). Mass excision from a lambda based library creates an equivalent cDNA library or other library in a plasmid vector. Plasmids excised from, for example, the lambda.Vtk cDNA library will contain the vaccinia tk sequences flanking the cDNA inserts or other insert DNAs, such as polynucleotides encoding immunoglobulin subunit polypeptides. This plasmid DNA can then be used to construct vaccinia recombinants by trimolecular recombination. Another embodiment of this method is to purify the lambda DNA directly from the initial lambda.Vtk library, and to transfect this recombinant viral (lambda) DNA or fragments thereof together with the two large vaccinia virus DNA fragments for trimolecular recombination.

Generation of vaccinia arms in vivo. Purification and transfection of vaccinia DNA or other virus DNA “arms” or fragments is a limiting factor in the construction of polynucleotide libraries by trimolecular recombination. Modifications to the method to allow for the requisite generation of virus arms, in particular vaccinia virus arms, in vivo would allow for more efficient construction of libraries in eukaryotic viruses.

Host cells can be modified to express a restriction endonuclease that recognizes a unique site introduced into a virus vector genome. For example, when a vaccinia virus infects these host cells, the restriction endonuclease will digest the vaccinia DNA, generating “arms” that can only be repaired, i.e., rejoined, by trimolecular recombination. Examples of restriction endonucleases include the bacterial enzymes NotI and ApaI, the Yeast endonuclease VDE (R. Hirata, Y. Ohsumi, A. Nakano, H. Kawasaki, K. Suzuki, Y. Anraku. 1990 J. Biological Chemistry 265: 6726-6733), the Chlamydomonas eugametos endonuclease I-CeuI and others well-known in the art. For example, a vaccinia strain containing unique NotI and ApaI sites in the tk gene has already been constructed, and a strain containing unique VDE and/or I-CeuI sites in the tk gene could be readily constructed by methods known in the art.

Constitutive expression of a restriction endonuclease would be lethal to a cell, due to the fragmentation of the chromosomal DNA by that enzyme. To avoid this complication, in one embodiment host cells are modified to express the gene(s) for the restriction endonuclease(s) under the control of an inducible promoter.

One method for inducible expression utilizes the Tet-On Gene Expression System (Clontech). In this system expression of the gene encoding the endonuclease is silent in the absence of an inducer (tetracycline). This makes it possible to isolate a stably transfected cell line that can be induced to express a toxic gene, i.e., the endonuclease (Gossen, M. et al., Science 268: 1766-1769 (1995)). The addition of the tetracycline derivative doxycycline induces expression of the endonuclease. In a preferred embodiment, BSC1 host cells will be stably transfected with the Tet-On vector controlling expression of the NotI gene. Confluent monolayers of these cells will be induced with doxycycline and then infected with v7.5/tk (unique NotI site in tk gene), and transfected with cDNA or insert DNA recombinant transfer plasmids or transfer DNA or lambda phage or phagemid DNA. Digestion of exposed vaccinia DNA at the unique NotI site, for example, in the tk gene or other sequence by the NotI endonuclease encoded in the host cells produces two large vaccinia DNA fragments which can give rise to full-length viral DNA only by undergoing trimolecular recombination with the transfer plasmid or phage DNA. Digestion of host cell chromosomal DNA by NotI is not expected to prevent production of modified infectious viruses because the host cells are not required to proliferate during viral replication and virion assembly.

In another embodiment of this method to generate virus arms such as vaccinia arms in vivo, a modified vaccinia strain is constructed that contains a unique endonuclease site in the tk gene or other non-essential gene, and also contains a heterologous polynucleotide encoding the endonuclease under the control of the T7 bacteriophage promoter at another non-essential site in the vaccinia genome. Infection of cells that express the T7 RNA polymerase would result in expression of the endonuclease, and subsequent digestion of the vaccinia DNA by this enzyme. In a preferred embodiment, the v7.5/tk strain of vaccinia is modified by insertion of a cassette containing the cDNA encoding NotI with expression controlled by the T7 promoter into the HindIII C or F region (Coupar, E. H. B. et al., Gene 68: 1-10 (1988); Flexner, C. et al., Nature 330: 259-262 (1987)), generating v7.5/tk/T7NotI. A cell line is stably transfected with the cDNA encoding the T7 RNA polymerase under the control of a mammalian promoter as described (O. Elroy-Stein, B. Moss. 1990 Proc. Natl. Acad. Sci. USA 87: 6743-6747). Infection of this packaging cell line with v7.5/tk/T7NotI will result in T7 RNA polymerase dependent expression of NotI, and subsequent digestion of the vaccinia DNA into arms. Infectious full-length viral DNA can only be reconstituted and packaged from the digested vaccinia DNA arms following trimolecular recombination with a transfer plasmid or phage DNA. In yet another embodiment of this method, the T7 RNA polymerase can be provided by co-infection with a T7 RNA polymerase recombinant helper virus, such as fowlpox virus (P. Britton, P. Green, S. Kottier, K. L. Mawditt, Z. Penzes, D. Cavanagh, M. A. Skinner. 1996 J. General Virology 77: 963-967).

A unique feature of trimolecular recombination employing these various strategies for generation of large virus DNA fragments, e.g., vaccinia DNA fragments in vivo is that digestion of the vaccinia DNA may, but does not need to precede recombination. It suffices that only recombinant virus escapes destruction by digestion. This contrasts with trimolecular recombination employing transfection of vaccinia DNA digested in vitro where, of necessity, vaccinia DNA fragments are created prior to recombination. It is possible that the opportunity for bimolecular recombination prior to digestion will yield a greater frequency of recombinants than can be obtained through trimolecular recombination following digestion.

Selection and Screening Strategies for Isolation of Recombinant Bispecific Antibodies Using Virus Vectors, Especially Poxviruses. In certain embodiments of the present invention, the trimolecular recombination method is used in the production of libraries of polynucleotides expressing immunoglobulin subunit polypeptides. In this embodiment, libraries comprising full-length immunoglobulin subunit polypeptides, or fragments thereof, are prepared by first inserting cassettes encoding immunoglobulin constant regions and signal peptides into a transfer plasmid which contains 5′ and 3′ regions homologous to vaccinia virus. In certain embodiments, the immunoglobulin subunit polypeptides have been modified to preferentially form bispecific bivalent antibodies; or they have been modified to form bispecific tetravalent antibodies; or they contain a recognition site for a modifying enzyme. Rearranged immunoglobulin variable regions are isolated by PCR from pre-B cells from unimmunized animals, from B cells or plasma cells from immunized animals, or from centroblasts or centrocytes derived from immune stimulated germinal centers of an immunized animal. These PCR fragments are cloned between, and in frame with the immunoglobulin signal peptide and constant region, to produce a coding region for an immunoglobulin subunit polypeptide. These transfer plasmids are introduced into host cells with poxvirus “arms,” and the tri-molecular recombination method is used to produce the libraries.

The present invention provides a variety of methods for identifying, i.e., selecting or screening for bispecific antibodies with a desired specificity, where the bispecific antibodies are produced in vitro in eukaryotic cells. These include screening the medium in which pools of host cells are grown for the presence of soluble bispecific antibodies with a desired antigenic specificity or a desired functional characteristic, or selecting for host cell effects such as antigen-induced cell death and antigen-induced signaling or screening pools of host cells for antigen-specific binding. It is to be understood that the identification techniques disclosed herein are directed to identifying bispecific antibodies which bind to at least two non-identical epitopes on one or more antigens. Thus, the identification techniques are designed to enrich for bispecific binding of the antibody which is, in all cases, confirmed by testing individually the specificity and crossreaction of antibodies produced by cells that express each pair of the immunoglobulin heavy and light chains isolated from cells producing the bispecific antibodies or, if monovalency is thought to be required, by testing antibodies produced by cells that express one pair of the isolated immunoglobulin heavy and light chains together with a second heavy chain, light chain or heavy and light chain combination that have arbitrarily selected specificities unrelated to the antigens of interest.

A screening method is provided to recover polynucleotides encoding bispecific antibodies, or antigen-binding fragments thereof, based on the antibody-antigen interaction resulting in detectable response. According to this method, pools of host cells are prepared which express fully-soluble bispecific antibodies as described herein. Expression is permitted, and the resulting cell medium is tested in various assays, the output of which require bispecific binding to two non-identical eiptopes. According to this method, the “function” being tested may be a standard effector function carried out by an immunoglobulin molecule, e.g., virus neutralization, opsonization, ADCC, antagonist/agonist activity, histamine release, hemagglutination, or hemagglutination inhibition. Alternatively, the “function” may simply refer to binding an antigen. In one embodiment described in more detail herein, the function is induction of a physiological response in a target cell, for example, apoptosis, proliferation, cytokine production, differentiation. This is especially advantageous when the one or more portions of a heterodimer receptor responsible for induction of the desired physiological response is unknown.

Referring to the first identification step as described above, a typical method to identify bispecific bivalent antibodies which induce an antigen-specific function may be carried out as follows. At least two different libraries of polynucleotides encoding diverse heavy chains from antibody producing cells of either naïve or immunized donors is constructed in a poxvirus vector such as a vaccinia virus vector, and a similarly diverse library of polynucleotides encoding immunoglobulin light chains is similarly constructed in vaccinia vector. In embodiments to identify bispecific bivalent antibodies, the two libraries of polynucleotides encoding immunoglobulin heavy chains, encode constant regions which comprise complementary heterodimerization domains. In embodiments to identify bispecific tetravalent antibodies, the heavy chain constant regions may have the CH2 region deleted.

The first library of polynucleotides encoding fully secreted immunoglobulin subunit polypeptides, e.g., either first heavy chain or light chain subunit polypeptides, is divided into a plurality of pools, as described above, each pool containing about 10, about 100, about 10³, about 10⁴, about 10⁵, about 10⁶, about 10⁷, about 10⁸, or about 10⁹ different polynucleotides encoding fully-secreted immunoglobulin subunit polypeptides with different variable regions. Preferred pools initially contain about 10³ polynucleotides each. Each pool is expanded and a replicate aliquot is set aside for later recovery. Where the pools of polynucleotides are constructed in virus vectors, e.g., poxvirus vectors such as vaccinia virus vectors, the pools are prepared, e.g., by diluting a high-titer stock of the virus library and using the portions to infect microcultures of tissue culture cells at a low MOI, e.g., MOI<0.1. Typically a greater than 1,000 fold expansion in the viral titer is obtained after 48 hrs infection. Expanding viral titers in multiple individual pools mitigates the risk that a subset of recombinants will be lost due to relatively rapid growth of a competing subset.

The virus pools are then used to infect pools of host cells equal to the number of virus pools prepared. The number of host cells infected with each pool depends on the number of polynucleotides contained in the pool, and the MOI desired. Virtually any host cell which is permissive for infection with the virus vector and which is capable of expressing fully-secreted immunoglobulin molecules may be used in this method. Such host cells include immunoglobulin-negative plasmacytoma cells, e.g., NS1 cells, Sp2/0 cells, or P3 cells, and early B-cell lymphoma cells as well as any of a large number of non-lymphoid cell lines, including fibroblastoid or epithelial cell lines such as HeLa cells or BSC1 cells that are permissive for infection by vaccinia virus. The choice of cell line will often be governed by any information available regarding the target antigens. Cell lines that do not express a target antigen or receptor for that antigen are preferred. The cells may be cultured in suspension or attached to a solid surface. Additional polynucleotides encoding immunoglobulin subunit polypeptides are also introduced into the host cell pools, for example, a library encoding second heavy chain subunit polypeptides, a library encoding first heavy chain subunit polypeptides or light chain subunit polypeptides depending on the type of polypeptide encoded by the first library, or one or more polynucleotides encoding known, fixed immunoglobulin subunit polypeptides which can combine with other known, fixed immunoglobulin subunit polypeptides to produce an antigen binding domain of a known specificity.

Expression of fully secreted bispecific (either bivalent or tetravalent) antibodies or fragments thereof is permitted. In certain embodiments, where the libraries encode monospecific immunoglobulins, the immunoglobulins are cross-linked in culture with an antibody or heavy and light chain subunits thereof, of known specificity.

The conditioned medium in which the host cell pools were cultured is then recovered and tested in a standardized assay for effector function in response to a specific target antigen, antigen binding, or physiological response in a target cell.

Any suitable assays may be used in this method. For example, the harvested cell supernatants may be tested for the ability to block or facilitate, i.e., act as an antagonist or an agonist of a target cellular function, for example, apoptosis, differentiation, functional activation or proliferation. Exemplary suitable assays are described in the Examples, infra. As used herein, an “assay” also includes simple detection of binding to a heterodimeric antigen, for example, through use of an ELISA assay, which is well known to those of ordinary skill in the art.

Where the conditioned medium in which a given host cell pool was grown elicits a desired signal in the assay of choice, the polynucleotides of the first library contained in host cells of that pool, as well as polynucleotides of any additional libraries being screened at the same time, are recovered from an aliquot of that pool previously set aside following initial expansion of that pool of polynucleotide.

To further enrich for polynucleotides of the libraries being screened which encode antigen-specific immunoglobulin subunit polypeptides, the polynucleotides recovered above are divided into a plurality of sub-pools. The sub-pools are set to contain fewer different members than the pools utilized above. For example, if each of the first pools contained 10³ different polynucleotides, the sub-pools are set up so as to contain, on average, about 10 or 100 different polynucleotides each. The sub-pools are introduced into host cells with additional libraries or fixed polynucleotides as above, and expression of fully secreted immunoglobulin molecules, or fragments thereof, is permitted. The conditioned medium in which the host cell pools are cultured is is recovered and tested in a standardized assay as described above, conditioned media samples which elicit the desired signal are identified, and the polynucleotides of the first library contained in host cells of that pool, as well as polynucleotides of any additional libraries being screened at the same time, are recovered from the aliquot previously set aside as described above. It will be appreciated by those of ordinary skill in the art that this process may be repeated one or more additional times in order to adequately enrich for polynucleotides encoding antigen-specific immunoglobulin subunit polypeptides.

Upon further enrichment steps for polynucleotides being screened for in the first identification step, and isolation of those polynucleotides, a similar process is carried out to recover polynucleotides of any subsequent libraries encoding immunoglobulin subunit polypeptides which, as part of an fully secreted immunoglobulin molecule, or fragment thereof, exhibits the desired antigen-specific function.

Pools of conditioned media may also be screened simply by assaying for antigen binding. Antigen binding may be detected by a variety of methods which are amenable to detection of bispecific antibody binding to two non-identical epitopes. For example, if two non-identical epitopes are bound to an enzyme and a substrate for that enzyme, respectively, binding of the bispecific antibody to both epitopes will bring the enzyme in proximity with the substrate, and enzyme reaction products are detected at concentrations of enzyme and substrate that would otherwise be suboptimal. Alternatively, one epitope may be bound to a substrate and the second epitope may be bound to a fluorescent tag such that only bispecific antibodies will both bind to the antigen coupled substrate and also bind the fluorescent molecule so as to evince a fluorescent signal.

The invention also provides methods to identify bispecific antibodies, or antigen-binding fragments thereof expressed in eukaryotic cells on the basis of either antigen-induced cell death, antigen-induced signaling, antigen binding, or other antigen-related functions. These methods are carried out essentially as described in U.S. Patent Application Publication No. 2002/0155447 A1, published Oct. 24, 2002 (U.S. Ser. No. 09/824,787, filed Apr. 4, 2001) which is incorporated herein by reference in its entirety.

In one embodiment, a selection method is provided to select polynucleotides encoding immunoglobulin molecules, or antigen-binding fragments thereof, based on direct antigen-induced apoptosis of the host cells. This method is carried out essentially as described in U.S. Patent Application Publication No. 2002/0155447 A1, published Oct. 24, 2002 (U.S. Ser. No. 09/824,787, filed Apr. 4, 2001) which is incorporated herein by reference in its entirety. Following infection and/or transfection with the various polynucleotide libraries and fixed polynucleotides as described above, synthesis and assembly of antibody molecules is allowed to proceed for a time period ranging from about 5 hours to about 48 hours, preferably for about 6 hours, about 10 hours, about 12 hours, about 16 hours about 20 hours, about 24 hours about 30 hours, about 36 hours, about 40 hours, or about 48 hours, even more preferably for about 12 hours or for about 24 hours; at which time the host cells are contacted with an antigen or antigens comprising at least two epitopes both of which must be bound, in order to cross-link any specific bispecific immunoglobulin receptors (i.e., membrane-bound immunoglobulin molecules, or antigen-binding fragments thereof) and induce apoptosis in those immunoglobulin expressing host cells which directly respond to cross-linking of antigen-specific immunoglobulin by induction of growth inhibition and apoptotic cell death. Host cells which have undergone apoptosis, or their contents, including the polynucleotides encoding an immunoglobulin subunit polypeptide which are contained therein, are recovered, thereby enriching for polynucleotides of the first library which encode a first immunoglobulin subunit polypeptide which, as part of an immunoglobulin molecule, or antigen-binding fragment thereof, specifically binds the antigen of interest. Further selection and enrichment steps are carried out, and additional identification steps are carried out if needed.

According to this method, host cells which express antigen-specific immunoglobulins on their surface are selected upon undergoing apoptosis. For example, if the host cells are attached to a solid substrate, those cells which undergo apoptosis are released from the substrate and the cells are recovered by harvesting the liquid medium in which the host cells are cultured. Alternatively, the host cells are attached to a solid substrate, and those cells which undergo apoptosis undergo a lytic event, thereby releasing their cytoplasmic contents into the liquid medium in which the host cells are cultured. Virus particles released from these cells can then be harvested in the liquid medium.

In utilizing this method, any host cell which is capable of expressing immunoglobulin molecules, or antigen-binding fragments thereof, on its surface may be used. Suitable host cells include immunoglobulin-negative plasmacytoma cell lines. Examples of such cell lines include, but are not limited to, an NS1 cell line, an Sp2/0 cell line, and a P3 cell line. Other suitable cell lines of either lymphoid or non-lymphoid origin, e.g. HeLa cells or BSC1 cells, that can be infected with and support expression of recombinant genes in vaccinia virus will be apparent to those of ordinary skill in the art.

In another embodiment, a screening method is provided to recover polynucleotides encoding bispecific antibodies, or antigen-binding fragments thereof, based on antigen-induced cell signaling. This method is carried out essentially as described in U.S. Patent Application Publication No. 2002/0155447 A1, published Oct. 24, 2002 (U.S. Ser. No. 09/824,787, filed Apr. 4, 2001) which is incorporated herein by reference in its entirety. According to this method, host cells are transfected with an easily detected reporter construct, for example luciferase, operably associated with a transcriptional regulatory region which is upregulated as a result of surface immunoglobulin crosslinking. Pools of host cells expressing bispecific antibodies or fragments thereof on their surface are contacted with antigen, and upon cross linking, the signal is detected in that pool.

The virus pools are used to infect pools of host cells equal to the number of virus pools prepared. These host cells have been engineered to express a reporter molecule as a result of surface immunoglobulin crosslinking. Necessary additional polynucleotides, either in library form or fixed, are added as well, and bispecific antibody expression is permitted.

The host cell pools are then contacted with a desired antigen under conditions wherein host cells expressing bispecific antibodies on their surface express the detectable reporter molecule upon cross-linking of said antibodies by at least two non-identical epitopes of the antigen, and the various pools of host cells are screened for expression of the reporter molecule. Those pools of host cells in which reporter expression is detected are harvested, and the polynucleotides of the one or more libraries contained therein are recovered from the aliquot previously set aside following initial expansion of that pool of polynucleotides. Enrichment and further identification steps are carried out in a similar manner.

In yet another embodiment, a selection or screening method is provided to select polynucleotides encoding bispecific antibodies, or antigen-binding fragments thereof, based on antigen-specific binding. According to this method, host cells which express antigen-specific immunoglobulin molecules, or fragments thereof on their surface are recovered based solely on the detection of antigen binding. In this embodiment, antigen binding is utilized as a selection method, i.e., where host cells expressing antigen-specific immunoglobulin molecules are directly selected by virtue of binding antigen, by methods similar to those described for selection based on cell death as described above. For example, if an antigen is bound to a solid substrate, host cells in suspension which bind the antigen may be recovered by binding, through the antigen, to the solid substrate. Binding of these same cells to a second soluble antigen with a fluorescent tag could then be detected by evincing a fluorescent signal. This method is carried out essentially as described in U.S. Patent Application Publication No. 2002/0155447 A1, published Oct. 24, 2002 (U.S. Ser. No. 09/824,787, filed Apr. 4, 2001) which is incorporated herein by reference in its entirety.

Alternatively, antigen binding may be used as a screening process, i.e., where pools of host cells are screened for detectable antigen binding by methods similar to that described above for antigen-induced cell signaling. For example, pools of host cells expressing immunoglobulins or fragments thereof on their surface are contacted with antigen, and antigen binding in a given pool is detected through an immunoassay, for example, through detection of an an enzyme-antigen conjugate or, indirectly, through detection of an enzyme-antibody conjugate which binds to the antigen. In a preferred embodiment, binding to two different antigens is detected by employing two different enzyme conjugates, or one enzyme conjugate and one antigen with a fluorescent tag, or two antigens with two distinguishable fluorescent tags.

Referring to the first step in the immunoglobulin identification methods as described above, selection via the antigen-specific binding method may be carried out as follows. A host cell is selected for infection and/or transfection that is capable of high level expression of immunoglobulin molecules on its surface. Preferably, the host cell grows in suspension. Following infection with the first and any subsequent polynucleotide libraries as described above as well as any fixed polynucleotides required, synthesis and assembly of antibody molecules is allowed to proceed. The host cells are then transferred into microtiter wells which have antigen bound to their surface. Host cells which bind antigen thereby become attached to the surface of the well, and those cells that remain unbound are removed by gentle washing. Alternatively, host cells which bind antigen may be recovered, for example, by fluorescence-activated cell sorting (FACS). FACS, also called flow cytometry, is used to sort individual cells on the basis of optical properties, including fluorescence. It is useful for screening large populations of cells in a relatively short period of time. Finally the host cells which bound to the antigen are recovered, thereby enriching for polynucleotides of the first library which encode one or more immunoglobulin subunit polypeptides which, as part of a bispecific antibody, or antigen-binding fragment thereof, specifically binds the antigen of interest. Further enrichment and additional identification steps are carried out as necessary employing a second antigen with a fluorescent tag. If the fluorescent tags evince distinguishable signals, then they may employed simultaneously. Otherwise, the same fluorescent tag on two different antigens may be employed sequentially to identify cells that express bispecific antibodies.

Screening via the antigen-specific binding method may be carried out as follows. The libraries of polynucleotides to be screened, constructed in a virus vector encoding immunoglobulin subunit polypeptides, are divided into a plurality of pools by the method described above. The virus pools are then used to infect pools of host cells equal to the number of virus pools prepared. In this screening method, it is preferred that the host cells are adherent to a solid substrate. Any additional libraries or fixed polynucleotides are also introduced into the host cell pools, and expression of bispecific antibodies or fragments thereof on the surface of the host cells is permitted. Detection of binding of a bispecific antibody to at least two non-identical epitopes is carried out as described above. Further enrichment and additional identification steps are carried out as necessary.

An antigen of interest may be contacted with bispecific antibodies by any convenient method, including various methods described herein. For example, in certain embodiments, antigens are attached to a solid substrate. As used herein, a “solid support” or a “solid substrate” is any support capable of binding a cell or antigen, which may be in any of various forms, as is known in the art. Well-known supports include tissue culture plastic, glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration as long as the coupled molecule is capable of binding to a cell. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. The support configuration may include a tube, bead, microbead, well, plate, tissue culture plate, petri plate, microplate, microtiter plate, flask, stick, strip, vial, paddle, etc., etc. A solid support may be magnetic or non-magnetic. Those skilled in the art will know many other suitable carriers for binding cells or antigens, or will be able to readily ascertain the same.

Alternatively, an antigen is expressed on the surface of an antigen-expressing presenting cell or “target cell.” As used herein an “target cell” refers to a cell which expresses an “antigen of interest” on its surface (i.e., an antigen or one or more antigens together comprising at least two non-identical epitopes) in a manner such that the antigen may interact with bispecific antibodies of the present invention. Certain target cells are engineered such that they express the antigen of interest as a recombinant protein, but the antigen may be a native antigen of that cell. Recombinant target cells may be constructed by any suitable method using molecular biology and protein expression techniques well-known to those of ordinary skill in the art. Typically, a plasmid vector which encodes the antigen of interest is transfected into a suitable cell, and the cell is screened for expression of the desired polypeptide antigen. Preferred recombinant target cells stably express the antigen of interest. A cell of the same type as the target cell except that it has not been engineered to express the antigen of interest is referred to herein as an “null-target cell.” Any suitable cell line may be used to prepare target cells. Examples of cell lines include, but are not limited to: monkey kidney CVI line transformed by SV40 (COS-7, ATCC CRL 1651); human embryonic kidney line (293, Graham et al. J. Gen Virol. 36:59 (1977)); baby hamster kidney cells (BHK, ATCC CCL 10); chinese hamster ovary-cells-DHFR(CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TR1 cells (Mather et al., Annals N.Y. Acad. Sci 383:44-68 (1982)); NIH/3T3 cells (ATCC CRL-1658); and mouse L cells (ATCC CCL-1). Additional cell lines will become apparent to those of ordinary skill in the art. A wide variety of cell lines are available from the American Type Culture Collection, 10801 University Boulevard, Manassas, Va. 20110-2209.

As will be appreciated by those of ordinary skill in the art, antigen-expressing target cells will comprise many naturally-occurring antigenic determinants on their surface in addition to the antigen of interest. Since host cells of the present invention express a broad spectrum of different immunoglobulin molecules, or antigen-specific fragments thereof it is to be expected that some of these antibodies will bind to these additional antigenic determinants. This background antibody binding is, however, rendered irrelevant by focusing on functional effects of the bispecific antibody, such as induction of proliferation, functional activation, apoptosis or differentiation of the target cells. In some embodiments, where one or more target molecules are known, it is possible to compare the functional effect of bispecific antibodies on untreated cells and cells in which expression of that target molecule has been suppressed by inhibitory RNA or, alternatively, on antigen negative cells and cells transfected with the gene or cDNA encoding the antigen of interest.

Kits. The present invention further provides a kit for the identification of antigen-specific recombinant bispecific antibodies expressed in a eukaryotic host cell. The kit comprises one or more containers filled with one or more of the ingredients required to carry out the methods described herein. A typical kit for the identification of bispecific, bivalent antibodies comprises three libraries constructed in eukaryotic virus vectors, e.g., vaccinia virus vectors: a first library of polynucleotides encoding, through operable association with a transcriptional control region, a plurality of either first heavy chain subunit polypeptides (where the constant region comprises a first heterodimerization domain) or light chain subunit polypeptides, a second library of polynucleotides encoding, through operable association with a transcriptional control region, a plurality of second heavy chain subunit polypeptides (where the constant region comprises a second heterodimerization domain which interacts with the first heterodimerization domain), and a third library of polynucleotides encoding, through operable association with a transcriptional control region, a plurality of immunoglobulin first heavy chain subunit polypeptides if the immunoglobulin subunit polypeptides encoded by the first library are light chains, or encoding a plurality of light chain subunit polyptides if the immunoglobulin subunit polypeptides encoded by the first library are first heavy chains.

A typical kit for the identification of bispecific, tetravalent antibodies comprises three libraries constructed in eukaryotic virus vectors, e.g., vaccinia virus vectors: a first library of polynucleotides encoding, through operable association with a transcriptional control region, a plurality of either CH2-deleted first heavy chain subunit polypeptides (where the constant region optionally comprises a first heterodimerization domain) or light chain subunit polypeptides, a second library of polynucleotides encoding, through operable association with a transcriptional control region, a plurality of CH2-deleted second heavy chain subunit polypeptides (where the constant region optionally comprises a second heterodimerization domain which interacts with the first heterodimerization domain), and a third library of polynucleotides encoding, through operable association with a transcriptional control region, a plurality of immunoglobulin first heavy chain subunit polypeptides if the immunoglobulin subunit polypeptides encoded by the first library are light chains, or encoding a plurality of light chain subunit polyptides if the immunoglobulin subunit polypeptides encoded by the first library are first heavy chains. Such a kit may also include an extracellular “means for tetramerization,” e.g., a third antibody which joins the first and second heavy chain subunit polypeptides.

A kit of the present invention may also include a population of host cells capable of expressing bispecific antibodies or fragments thereof. In certain embodiments a kit will further include control antigens (e.g., on a target cell) and reagents to standardize and validate the identification of particular antigens of interest.

Specific kits to identify bispecific antibodies directed to a known target antigen typically include one or more additional polynucleotides encoding fixed immunoglobulin subunit polypeptides, e.g., heavy or light chains, which contribute to a known antigen binding domain. Such a known antigen binding domain would be specific for one of two epitopes to be bound by a bispecific antibody to be identified. An alternative kit includes a plurality of immunoglobulin subunit polypeptides with a defined specificity, which generally form antigen binding domains which bind a known antigen, but not necessarily the exact same epitope. Such additional polynucleotides may be provided in eukaryotic virus vectors or any other suitable vector.

In these kits, the various libraries may be provided both as infectious virus particles and as inactivated virus particles, where the inactivated virus particles are capable of infecting the host cells and allowing expression of the polynucleotides contained therein, but the inactivated viruses do not undergo virus replication.

Use of a kit of the present invention is in accordance to the methods described herein.

Isolated antibodies, host cells and polynucleotides. The present invention further provides an isolated antigen-specific bispecific antibody, e.g., a bispecific bivalent antibody or a bispecific tetravalent antibody as described herein, or antigen binding fragment thereof, produced by any of the methods disclosed herein. Such isolated bispecific antibodies may be useful as diagnostic or therapeutic reagents. Further provided is a composition comprising an isolated bispecific antibody of the present invention, and a pharmaceutically acceptable carrier.

Further provided are methods of producing polynucleotides encoding a multispecific, e.g., bispecific antibody of the present invention, which method includes combining polynucleotides identified and isolated according to the methods of the present invention. In addition, methods of producing a host cell which expresses a bispecific bivalent antibody or a bispecific tetravalent antibody are provided, such methods including introducing the polynucleotides produced as above into a host cell. The invention further includes polynucleotides produced as above, and host cells produced as above. The invention also provides a method of producing a multispecific, e.g., bispecific antibody of the present invention by culturing the host cell produced as above, and recovering the antibody.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984).

### EXAMPLES

### Example 1

### Construction of Human Bispecific Antibody Libraries of Diverse Specificity

Libraries of polynucleotides encoding diverse immunoglobulin subunit polypeptides are produced as follows. Genes for human VH (variable region of heavy chain), V-Kappa (variable region of kappa light chain) and V-Lambda (variable region of lambda light chains) are amplified by PCR. For each of the three variable gene families, both a recombinant plasmid library and a vaccinia virus library is constructed. The variable region genes are inserted into a pH5/tk or p7.5/tk-based transfer/expression plasmid between immunoglobulin leader and constant region sequences (suitably modified to comprise a heterodimerization domain or a means for tetramerization) of the corresponding heavy chain or light chain. This plasmid is employed to generate the corresponding vaccinia virus recombinants by trimolecular recombination and can also be used directly for high level expression of immunoglobulin chains following transfection into vaccinia virus infected cells. Cells are first infected with a vaccinia heavy chain library, followed by transient transfection with a plasmid light chain library. The co-expression of IgM or IgG1 heavy chain and light chain results in the assembly and expression of antibody molecules. This example describes the construction o libraries suitable for identification of standard monospecific bivalent antibodies. These methods are illustrative of methods which are used to construct bispecific antibodies.

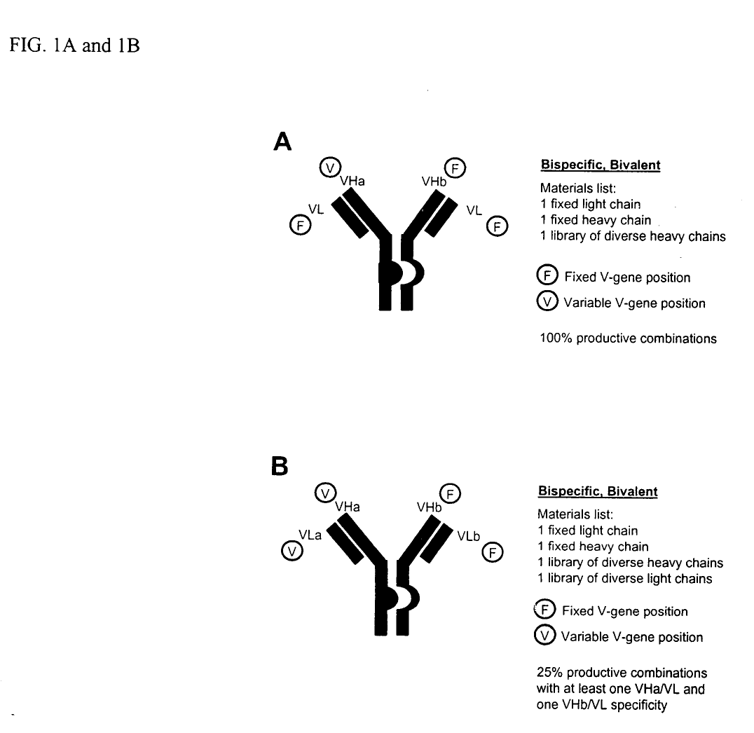

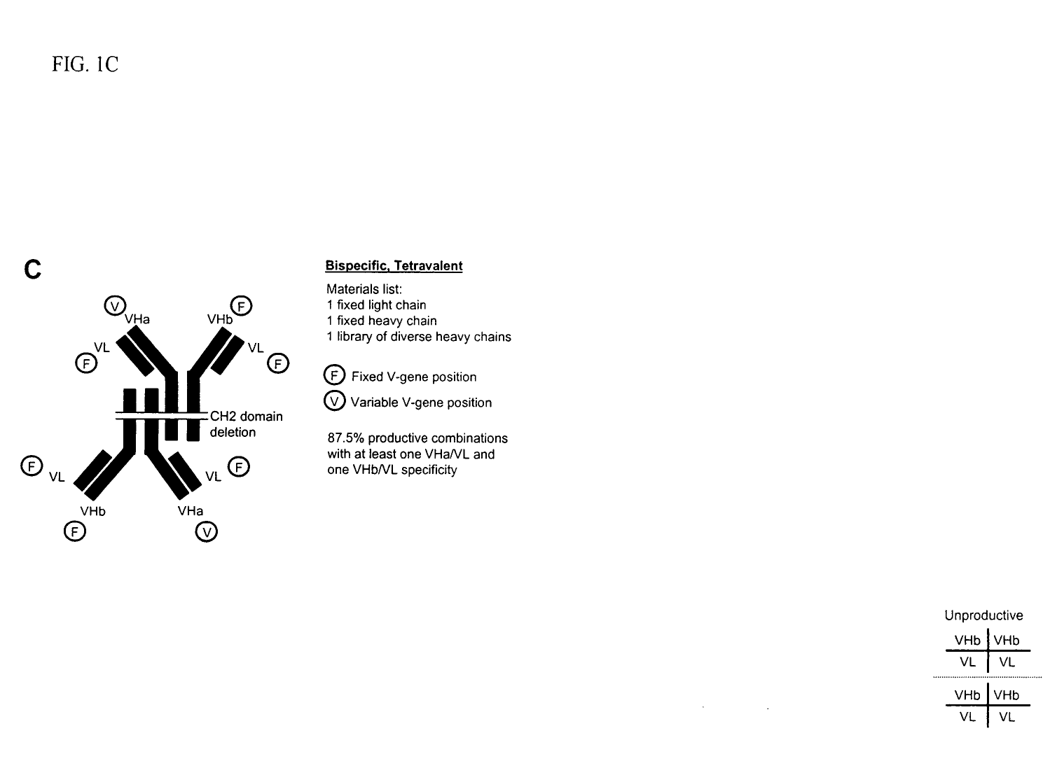

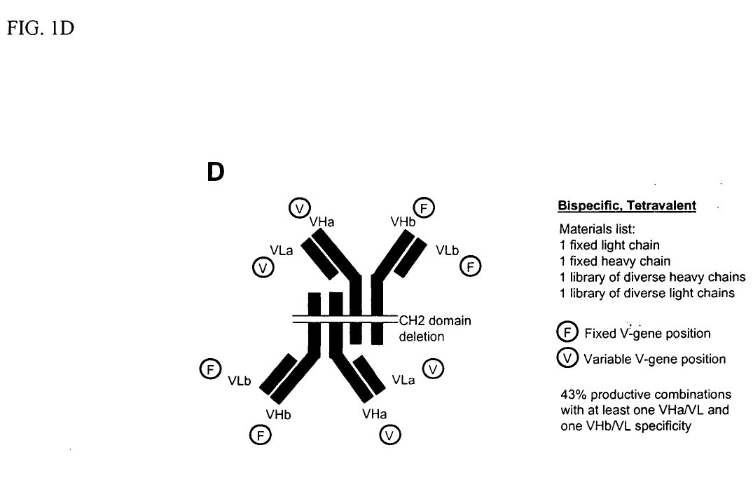

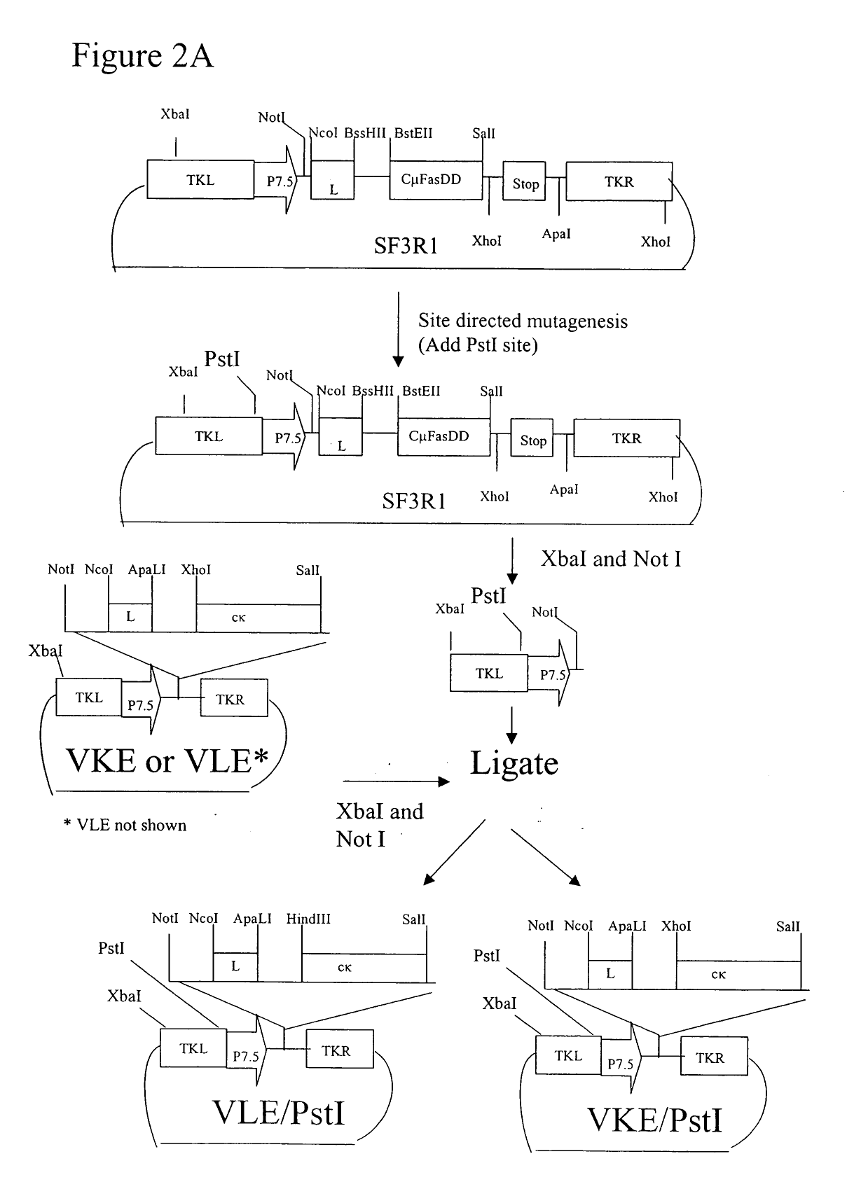

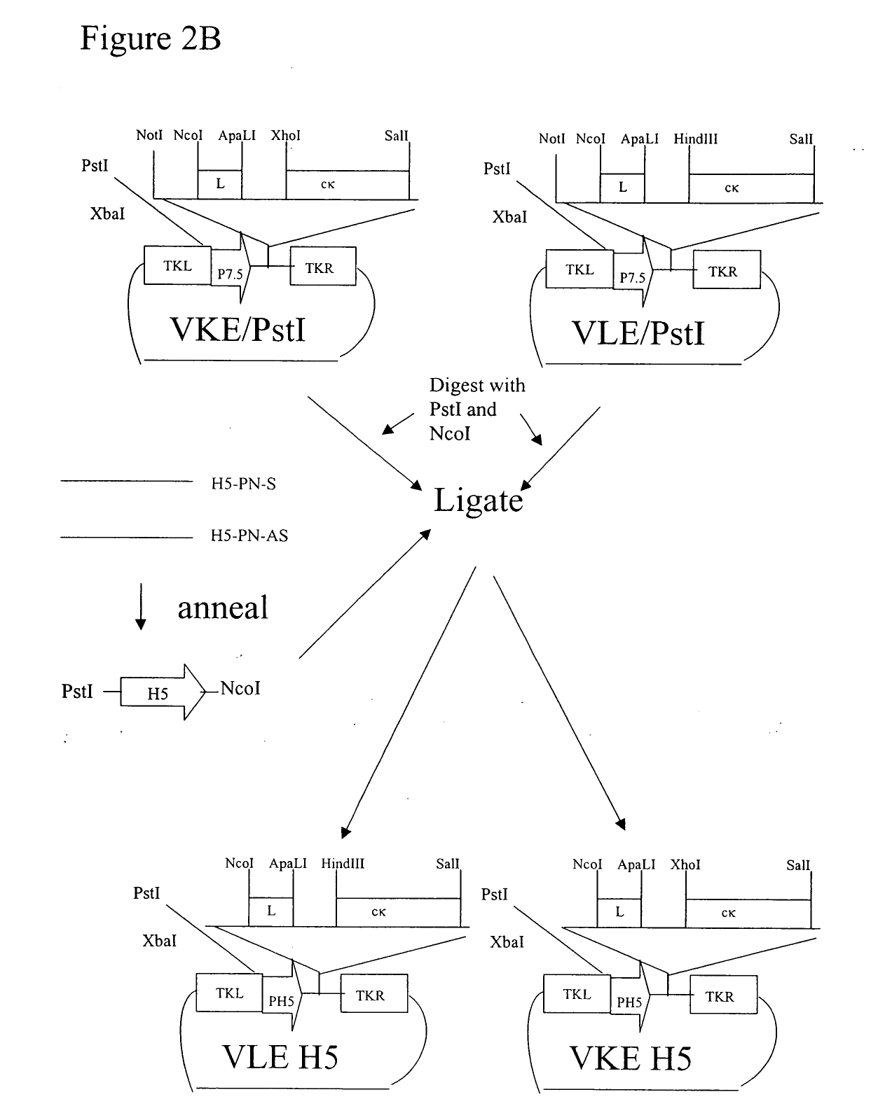

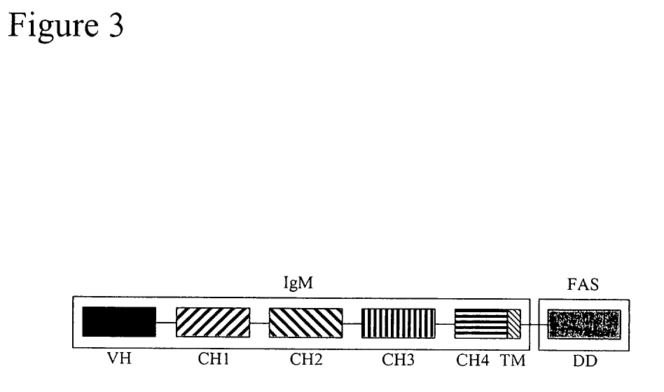

1.1 pVKE-H5 and pVLE-H5. Expression vectors comprising the vaccinia H5 promoter and the human kappa and lambda immunoglobulin light chain constant regions, designated herein as pVKE H5 and pVLE H5, are constructed as follows. The strategy is depicted in FIGS. 2A and 2B. Cloning began with the creation of a variant of pVKE and pVLE (produced as described in Example 1 of U.S. Patent Application Publication No. U.S. Pat. No. 2,002,0018785A1, published Sep. 5, 2002 and PCT Publication WO 02102855, published Dec. 27, 2002, each incorporated herein by reference) that has been modified to contain the human μ membrane immunoglobulin constant region coupled in frame with the Fas Death Domain according to the following protocol. This construct is formally designated SF3R1 (FIG. 3).

Strategy for Assembly of SF3R1;

I. Digest pVHE with BstEII and SalI and gel purify (˜1.4 Kb).
II. PCR amplify pVHE fragment with CH4(F) and CH4(R2) and gel purify the product (356 nt).
III. PCR amplify pBS-APO14.2 with FAS(F3) and FAS(R) and gel purify the product (440 nt).
IV. Use the fragments from steps II and III in a PCR reaction in combination with CH4(F) and FAS(R) (vary the concentration of the fragments from II and III) (795 nt).
V. Digest the gel purified PCR product with SacII and SalI, gel purify again (780 nt).
VI. Digest original pVHE with with SacII and SalI, gel purify again (˜6.8 Kb).

VII. Ligate fragments from V and VI.

Primers used in the assembly of SF3R1;
CH4(F)-

(SEQ ID NO:14)

5′ CTCTCCCGCGGACGTCTTCGT 3′
CH4(R2)-

(SEQ ID NO:15)

5′ AATAGTGGTGATATATTTCACCTTGAACAA 3′
FAS(F3)-

(SEQ ID NO:16)

5′ TTGTTCAAGGTGAAAGTGAAGAGAAAGGAA 3′
FAS(R)-

(SEQ ID NO:17)

5′ ACGCGTCGACCTAGACCAAGCTTTGGATTTCAT 3′

Expression of this molecule is controlled by the vaccinia 7.5 promoter. This construct contains unique XbaI and NotI restriction endonuclease sites 5′ (XbaI) and 3′ (NotI) of the 7.5 promoter and leader sequence (See FIG. 2A). This construct was mutagenized using the Gene Editor Kit from Promega (Q9280) to incorporate a unique PstI site 5′ of the 7.5 promoter. The primers used for mutagenesis were:

| | |
|---|---|
| pstmutF- | |
| 5′ GTCGAATAAAGTGAACAATAATTAATTCT [symbol] A [symbol] | (SEQ ID NO:18) |
| TGTCATCATGGCGGCC 3′ | |
| pstmutR-5′ | |
| 5′ GGCCGCCATGATGACA [symbol] T [symbol] AGAATTAATTATT | (SEQ ID NO:19) |
| GTTCACTTTATTCGAC 3′ | |

where the nucleotides in bold and italics represent the introduced mutations. After clones were identified and sequence verified to contain the new PstI site, the entire 5′ region including most of the TKL and the 7.5 promoter were removed by digestion with XbaI and NotI. This fragment was gel purified and ligated into the XbaI and NotI sites of pVKE and pVLE, creating pVKE/PstI and pVLE/PstI.

The H5 promoter was constructed as a custom oligo and was created to have the correct overhangs to facilitate ligation into PstI and NcoI (NcoI is immediately 3′ of the NotI site) of pVKE/PstI and pVLE/PstI (FIG. 2B). The oligos used were:

| | |
|---|---|
| H5-PN-S | |
| 5′ GAAAAAATGAAAATAAATACAAAGGTTCTTGAG | (SEQ ID NO:20) |
| GGTTGTGTTAAATTGAAAGCGAGAAATAATCATAAA | |
| TTC 3′ | |
| H5-PN-AS | |
| 5′ CATGGAATTTATGATTATTTCTCGCTTTCAATT | (SEQ ID NO:21) |
| TAACACAACCCTCAAGAACCTTTGTATTTATTTTCA | |
| TTTTTTCTGCA 3′ | |

The pVKE/PstI and pVLE/PstI were linearized with PstI and NcoI and the H5 promoter containing oligos were annealed together and then ligated into each vector. Insertion was verified by sequencing. The new transfer plasmids are designated pVKE H5 and pVLE H5.

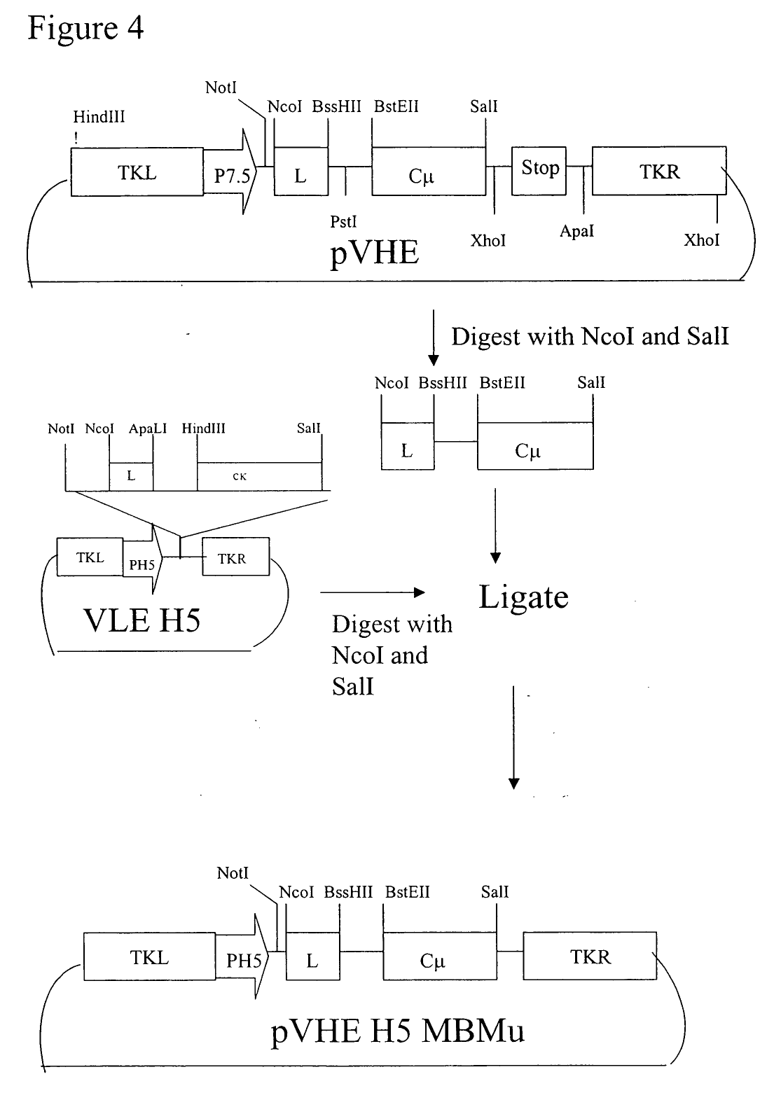

1.2 pVHE-H5 MBMu. An expression vector comprising the vaccinia H5 promoter and the human p membrane immunoglobulin constant region, designated herein as pVHE-H5 MBMu is constructed as follows. The strategy is depicted in FIG. 4. In order to create pVHE-H5 MBMu, the entire human μ membrane immunoglobulin constant region as well as the Ig leader sequence and Ig variable gene cloning region was excised from pVHE (produced as described in example 1 of U.S. Patent Application Publication No. U.S. Pat. No. 2,002,0018785A1, published Sep. 5, 2002 and PCT Publication WO 02102855, published Dec. 27, 2002, each incorporated herein by reference) by digestion with NcoI and SalI and inserted into the NcoI and SalI sites of pVLE H5. This manipulation results in the introduction of the entire human μ membrane immunoglobulin constant region as well as the Ig leader sequence and Ig variable gene cloning region from VHE replacing the leader sequence, variable gene insertion sites and kappa constant domain of pVLE H5. The resulting vector is designated VHE H5 MBMu

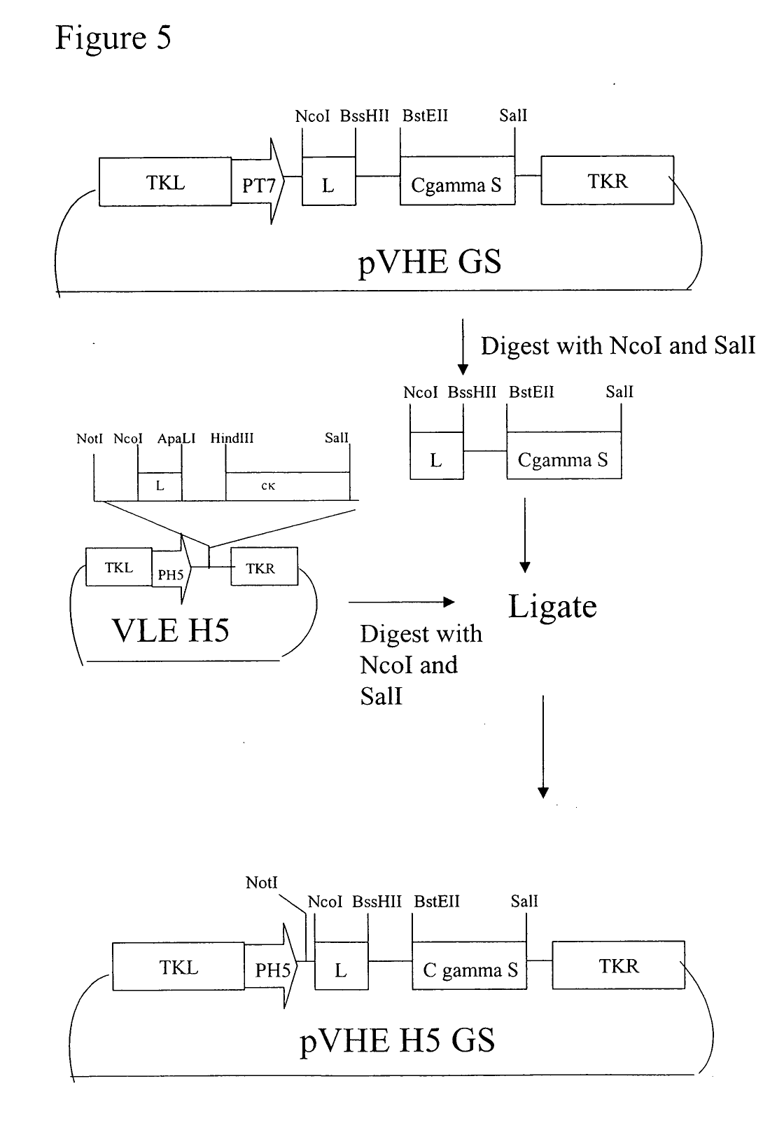

1.3 pVHE H5 GS. An expression vector comprising the vaccinia H5 promoter and the humanIgG1. secretory immunoglobulin constant region, designated herein as pVHE H5 GS is constructed as follows. The strategy is depicted in FIG. 5. In order to create pVHE-H5 GS, the entire human IgG1 secretory immunoglobulin constant region as well as the Ig leader sequence and Ig variable gene cloning region was excised from pVHE T7 GS (produced as described in example 1 of U.S. Patent Application Publication No. U.S. Pat. No. 2,002,0018785A1, published Sep. 5, 2002 and PCT Publication WO 02102855, published Dec. 27, 2002, each incorporated herein by reference) by digestion with NcoI and SalI and inserted into the NcoI and SalI sites of pVLE H5. This manipulation results in the introduction of the entire human IgG1 secretory immunoglobulin constant region as well as the Ig leader sequence and Ig variable gene cloning region from VHE T7 GS replacing the leader sequence, variable gene insertion sites and kappa constant domain of pVLE H5. The resulting vector is designated VHE H5 GS.

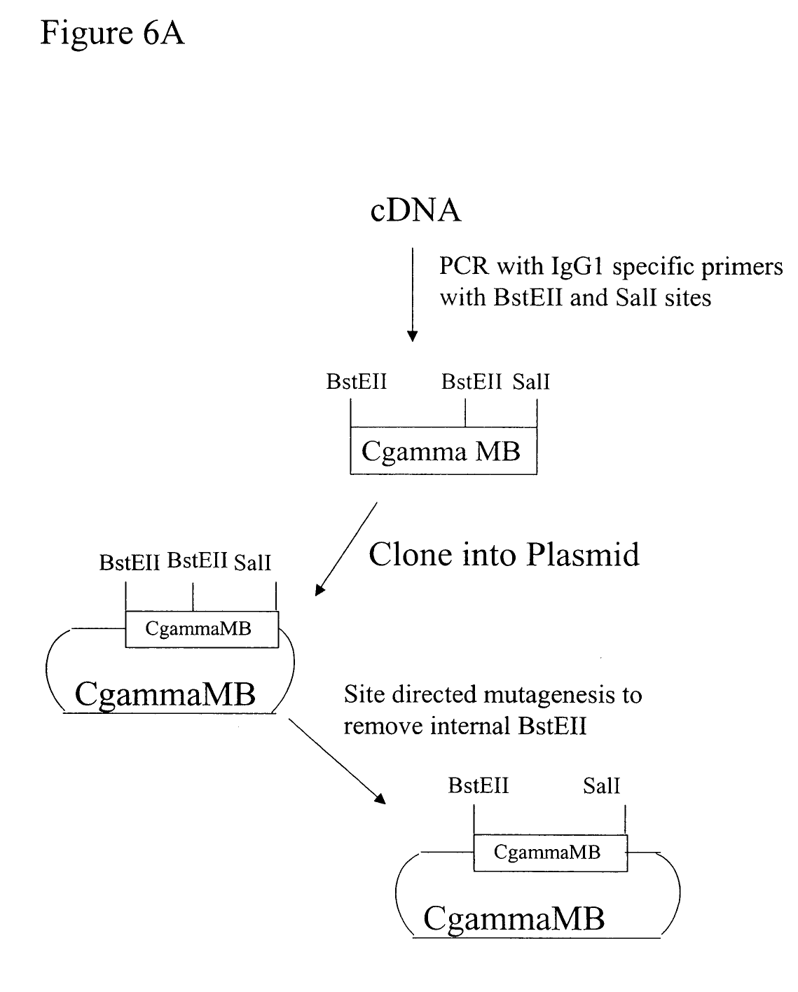

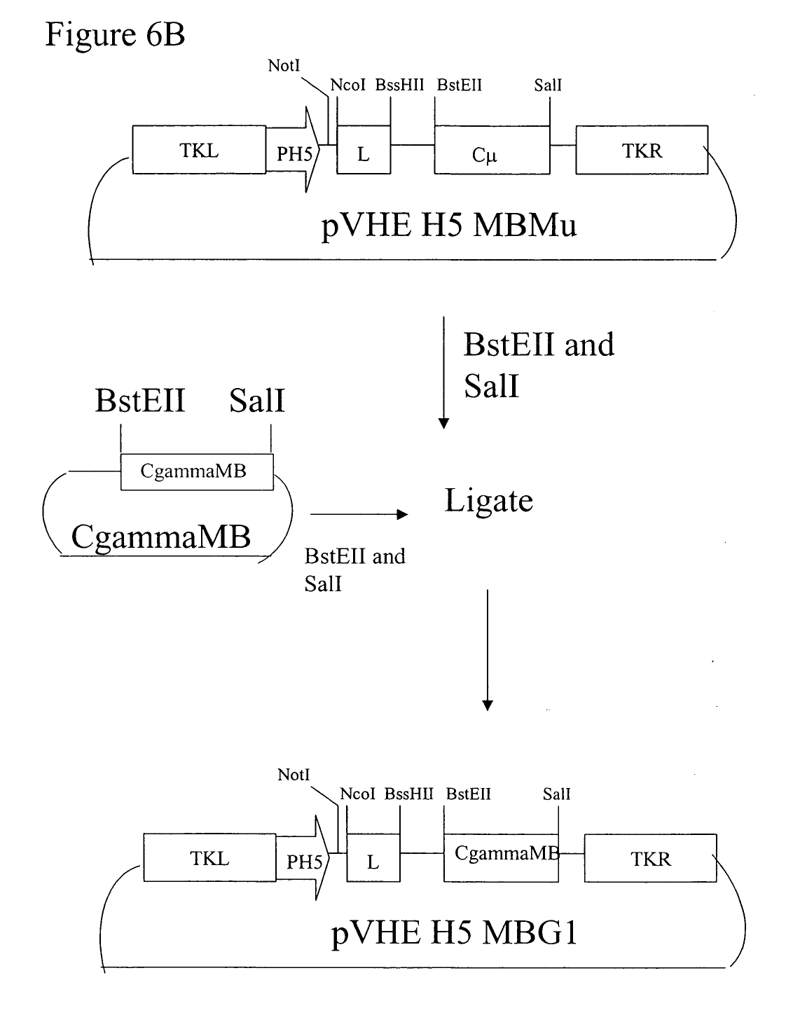

1.4 pVHE H5 MBG1. An expression vector comprising the vaccinia H5 promoter and the human IgG1 membrane immunoglobulin constant region, designated herein as pVHE-H5 MBG1 is constructed as follows. The strategy is depicted in FIGS. 6A and 6B. The entire constant domain of membrane bound IgG1 was initially cloned into pBluescript KS+ from pooled BLCL and bone marrow cDNA using the following PCR primers;

| | |
|---|---|
| C-gamma1F: | |
| 5′-ATTAGGATCCGGTCACCGTCTCCTCAGCC | (SEQ ID NO:22) |
| C-gamma1R: | |
| 5′-ATTAGTCGACCTAGGCCCCCTGTCCGATCAT | (SEQ ID NO:23) |

The IgG1 insert was then mutagenized to destroy an internal BstEII site to facilitate insertion into pVHE H5 MBMu using a flanking BstEII site. This was accompished using a QuickChange XL site-Directed Mutagenesis kit (Stratagene) and the following PCR primers;

| | |
|---|---|
| BstEIImutF: | |
| 5′ CAGCGTGGTGAC [symbol] GTGCCCTCCAGCAG 3′ | (SEQ ID NO:24) |
| BstEIImutR: | |
| 5′ CTGCTGGAGGGCAC [symbol] GTCACCACGCTG 3′ | (SEQ ID NO:25) |

where the nucleotides in bold and italics represent the introduced mutation. The mutagenized membrane bound IgG1 clone was digested with SalI and BstEII to release the constant domain. The fragment was gel isolated. In parallel plasmid pVHE H5 MBMu was digested with SalI and BstEII to remove a similar insert and the remaining vector was gel purified. The two pieces were then ligated according to standard protocol. Positive clones were identified and sequenced. The new designation for this clone was pVHE H5 MBG1.

The full sequence and annotation of each of the H5-driven Ig cloning vectors described above are designated herein as VLE-H5 (SEQ ID NO:26, lambda constant region sequence, SEQ ID NO:27), VKE-H5 (SEQ ID NO:28, kappa constant region sequence SEQ ID NO:29), VHE-H5MBG (SEQ ID NO:30, membrane bound IgG1 constant domain sequence SEQ ID NO:31), VHE-H5MBMu (SEQ ID NO:32, membrane bound IgM constant domain sequence, SEQ ID NO:33), and VHE-H5GS (SEQ ID NO:34, secreted IgG constant domain sequcne, SEQ ID NO:35).

1.5 Variable Regions. Heavy chain, kappa light chain, and lambda light chain variable regions are isolated by PCR for cloning in the expression vectors produced as described above, by the following method. RNA isolated from normal human bone marrow pooled from multiple donors (available from Clontech) is used for cDNA synthesis. Aliquots of the cDNA preparations are used in PCR amplifications with primer pairs selected from the following sets of primers: VH/JH, V-Kappa/J-Kappa or V-Lambda/J-Lambda. The primers used to amplify variable regions are listed in Tables 1 and 2.

(a) Heavy chain variable regions. Due to the way the plasmid expression vectors were designed, VH primers, i.e., the forward primer in the pairs used to amplify heavy chain V regions, have the following generic configuration, with the BssHII restriction site in bold:
VH primers: GCGCGCACTCC-start of VH FR1 primer (SEQ ID NO:36).

The primers are designed to include codons encoding the last 4 amino acids in the leader, with the BssHII site coding for amino acids −4 and −3, followed by the VH family-specific FR1 sequence. Tables 1 and 2 lists the sequences of the different family-specific VH primers. Since the last 5 amino acids of the heavy chain variable region, i.e., amino acids 109-113, which are identical among the six human heavy chain J regions, are embedded in plasmid pVHE, JH primers, i.e., the reverse primers used to amplify the heavy chain variable regions, exhibit the following configuration to include a BstEII site, which codes for amino acids 109 and 110 (shown in bold):
JH primers: nucleotide sequence for amino acids 103-108 of VH (ending with a G)-GTCACC

Using these sets of primers, the VH PCR products start with the codons coding for amino acids −4 to 110 with BssHII being amino acids −4 and −3, and end at the BstEII site at the codons for amino acids 109 and 110. Upon digestion with the appropriate restriction enzymes, these PCR products are cloned into pVHE digested with BssHII and BstEII.

In order to achieve amplification of most of the possible rearranged heavy chain variable regions, families of VH and JH primers, as shown in Tables 1 and 2, are used. The VH1, 3, and 4 families account for 44 out of the 51 V regions present in the human genome. The embedding of codons coding for amino acids 109-113 in the expression vector precludes the use of a single common JH primer. However, the 5 JH primers shown in Tables 1 and 2 can be pooled for each VH primer used to reduce the number of PCR reactions required.

(b) Kappa light chain variable regions. The V-Kappa primers, i.e., the forward primer in the pairs used to amplify kappa light chain variable regions, have the following generic configuration, with the ApaLI restriction site in bold:

V-Kappa primer:
GTGCACTCC-start of V-Kappa FR1 primer

The V-Kappa primers contain codons coding for the last 3 amino acids of the kappa light chain leader with the ApaLI site coding for amino acids −3 and −2, followed by the V-Kappa family-specific FR1 sequences. Since the codons encoding the last 4 amino acids of the kappa chain variable region (amino acids 104-107) are embedded in the expression vector pVKE, the J-Kappa primers, i.e., the reverse primer in the pairs used to amplify kappa light chain variable regions, exhibit the following configuration:
J-Kappa primer: nucleotide sequence coding for amino acids 98-103 of V-Kappa-CTCGAG

The XhoI site (shown in bold) comprises the codons coding for amino acids 104-105 of the kappa light chain variable region. The PCR products encoding kappa light chain variable regions start at the codon for amino acid −3 and end at the codon for amino acid 105, with the ApaLI site comprising the codons for amino acids −3 and −2 and the XhoI site comprising the codons for amino acids 104 and 105. V-Kappa 1/4 and V-Kappa 3/6 primers each have two degenerate nucleotide positions. Employing these J-Kappa primers (see Tables 1 and 2), J-Kappa 1, 3 and 4 will have a Val to Leu mutation at amino acid 104, and J-Kappa 3 will have an Asp to Glu mutation at amino acid 105.

(c) Lambda light chain variable regions. The V-Lambda primers, i.e., the forward primer in the pairs used to amplify lambda light chain variable regions, have the following generic configuration, with the ApaLI restriction site in bold:

| | |
|---|---|
| V-Lambda primer: | GTGCACTCC-start of VL |

The ApaLI site comprises the codons for amino acids −3 and −2, followed by the V-Lambda family-specific FR1 sequences. Since the codons encoding the last 5 amino acids of V-Lambda (amino acids 103-107) are embedded in the expression vector pVLE, the J-Lambda primers exhibit the following configuration to include a HindIII site (shown in bold) comprising the codons encoding amino acids 103-104:

J-Lambda primer:
-nucleotide sequence for amino acids 97-102 of
VL-AAGCTT

The PCR products encoding lambda light chain variable regions start at the codon for amino acid −3 and end at the codon for amino acid 104 with the ApaLI site comprising the codons for amino acids −3 and −2, and HindIII site comprising the codons for amino acids 103 and 104.

TABLE 1

Oligonucleotide primers for PCR amplification
of human immunoglobulin variable regions.
Recognition sites for restriction enzymes
used in cloning are indicated in bold type.
Primer sequences are from 5′ to 3′.

| | | |
|---|---|---|
| VH1 | (SEQ ID NO:37) | TTT TGC GCG CAC TCC |
| | | CAG GTG CAG CTG GTG |
| | | CAG TCT GG |
| VH2 | (SEQ ID NO:38) | AATA TGC GCG CAC TCC |
| | | CAG GTC ACC TTG AAG |
| | | GAG TCT GG |
| VH3 | (SEQ ID NO:39) | TTT TGC GCG CAC TCC |
| | | GAG GTG CAG CTG GTG |
| | | GAG TCT GG |
| VH4 | (SEQ ID NO:40) | TTT TGC GCG CAC TCC |
| | | CAG GTG CAG CTG CAG |
| | | GAG TCG GG |
| VH5 | (SEQ ID NO:41) | AATA TGC GCG CAC TCC |
| | | GAG GTG CAG CTG GTG |
| | | CAG TCT G |
| JH1 | (SEQ ID NO:42) | GAC GGT GAC CAG GGT |
| | | GCC CTG GCC CCA |
| JH2 | (SEQ ID NO:43) | GAC GGT GAC CAG GGT |
| | | GCC ACG GCC CCA |
| JH3 | (SEQ ID NO:44) | GAC GGT GAC CAT TGT |
| | | CCC TTG GCC CCA |
| JH4/5 | (SEQ ID NO:45) | GAC GGT GAC CAG GGT |
| | | TCC CTG GCC CCA |
| JH6 | (SEQ ID NO:46) | GAC GGT GAC CGT GGT |
| | | CCC TTG GCC CCA |
| V-Kappa 1 | (SEQ ID NO:47) | TTT GTG CAC TCC GAC |
| | | ATC CAG ATG ACC CAG |
| | | TCT CC |
| V-Kappa 2 | (SEQ ID NO:48) | TTT GTG CAC TCC GAT |
| | | GTT GTG ATG ACT CAG |
| | | TCT CC |
| V-Kappa 3 | (SEQ ID NO:49) | TTT GTG CAC TCC GAA |
| | | ATT GTG TTG ACG CAG |
| | | TCT CC |
| V-Kappa 4 | (SEQ ID NO:50) | TTT GTG CAC TCC GAC |
| | | ATC GTG ATG ACC CAG |
| | | TCT CC |
| V-Kappa 5 | (SEQ ID NO:51) | TTT GTG CAC TCC GAA |
| | | ACG ACA CTC ACG CAG |
| | | TCT CC |
| V-Kappa 6 | (SEQ ID NO:52) | TTT GTG CAC TCC GAA |
| | | ATT GTG CTG ACT CAG |
| | | TCT CC |
| J-Kappa 1 | (SEQ ID NO:53) | GAT CTC GAG CTT GGT |
| | | CCC TTG GCC GAA |
| J-Kappa 2 | (SEQ ID NO:54) | GAT CTC GAG CTT GGT |
| | | CCC CTG GCC AAA |
| J-Kappa 3 | (SEQ ID NO:55) | GAT CTC GAG TTT GGT |
| | | CCC AGG GCC GAA |
| J-Kappa 4 | (SEQ ID NO:56) | GAT CTC GAG CTT GGT |
| | | CCC TCC GCC GAA |
| J-Kappa 5 | (SEQ ID NO:57) | AAT CTC GAG TCG TGT |
| | | CCC TTG GCC GAA |
| V-Lambda 1 | (SEQ ID NO:58) | TTT GTG CAC TCC CAG |
| | | TCT GTG TTG ACG CAG |
| | | CCG CC |
| V-Lambda 2 | (SEQ ID NO:59) | TTT GTG CAC TCC CAG |
| | | TCT GCC CTG ACT CAG |
| | | CCT GC |
| V-Lambda 3A | (SEQ ID NO:60) | TTT GTG CAC TCC TCC |
| | | TAT GTG CTG ACT CAG |
| | | CCA CC |
| V-Lambda 3B | (SEQ ID NO:61) | TTT GTG CAC TCC TCT |
| | | TCT GAG CTG ACT CAG |
| | | GAC CC |
| V-Lambda 4 | (SEQ ID NO:62) | TTT GTG CAC TCC CAC |
| | | GTT ATA CTG ACT CAA |
| | | CCG CC |
| V-Lambda 5 | (SEQ ID NO:63) | TTT GTG CAC TCC CAG |
| | | GCT GTG CTC ACT CAG |
| | | CCG TC |
| V-Lambda 6 | (SEQ ID NO:64) | TTT GTG CAC TCC AAT |
| | | TTT ATG CTG ACT CAG |
| | | CCC CA |
| V-Lambda 7 | (SEQ ID NO:65) | TTT GTG CAC TCC CAG |
| | | GCT GTG GTG ACT CAG |
| | | GAG CC |
| J-Lambda 1 | (SEQ ID NO:66) | GGT AAG CTT GGT CCC |
| | | AGT TCC GAA GAC |
| J-Lambda 2/3 | (SEQ ID NO:67) | GGT AAG CTT GGT CCC |
| | | TCC GCC GAA T |

TABLE 2

Oligonucleotide primers for PCR amplification
of human immunoglobulin variable regions.
Recognition sites for restriction enzymes
used in cloning are indicated in bold type.
Primer sequences are from 5′ to 3′.

| | | |
|---|---|---|
| VH1a | (SEQ ID NO:68) | AATA TGC GCG CAC TCC |
| | | CAG GTG CAG CTG GTG |
| | | CAG TCT GG |
| VH2a | (SEQ ID NO:69) | AATA TGC GCG CAC TCC |
| | | CAG GTC ACC TTG AAG |
| | | GAG TCT GG |
| VH3a | (SEQ ID NO:70) | AATA TGC GCG CAC TCC |
| | | GAG GTG CAG CTG GTG |
| | | GAG TCT GG |
| VH4a | (SEQ ID NO:71) | AATA TGC GCG CAC TCC |
| | | CAG GTG CAG CTG CAG |
| | | GAG TCG GG |
| VH5a | (SEQ ID NO:72) | AATA TGC GCG CAC TCC |
| | | GAG GTG CAG CTG GTG |
| | | CAG TCT G |
| JH1a | (SEQ ID NO:73) | GA GAC GGT GAC CAG GGT |
| | | GCC CTG GCC CCA |
| JH2a | (SEQ ID NO:74) | GA GAC GGT GAC CAG GGT |
| | | GCC ACG GCC CCA |
| JH3a | (SEQ ID NO:75) | GA GAC GGT GAC CAT TGT |
| | | CCC TTG GCC CCA |
| JH4/5a | (SEQ ID NO:76) | GA GAC GGT GAC CAG GGT |
| | | TCC CTG GCC CCA |
| JH6a | (SEQ ID NO:77) | GA GAC GGT GAC CGT GGT |
| | | CCC TTG GCC CCA |
| V-Kappa 1a | (SEQ ID NO:78) | CAGGA GTG CAC TCC GAC |
| | | ATC CAG ATG ACC CAG |
| | | TCT CC |
| V-Kappa 2a | (SEQ ID NO:79) | CAGGA GTG CAC TCC GAT |
| | | GTT GTG ATG ACT CAG |
| | | TCT CC |
| V-Kappa 3a | (SEQ ID NO:80) | CAGGA GTG CAC TCC GAA |
| | | ATT GTG TTG ACG CAG |
| | | TCT CC |
| V-Kappa 4a | (SEQ ID NO:81) | CAGGA GTG CAC TCC GAC |
| | | ATC GTG ATG ACC CAG |
| | | TCT CC |
| V-Kappa 5a | (SEQ ID NO:82) | CAGGA GTG CAC TCC GAA |
| | | ACG ACA CTC ACG CAG |
| | | TCT CC |
| V-Kappa 6a | (SEQ ID NO:83) | CAGGA GTG CAC TCC GAA |
| | | ATT GTG CTG ACT CAG |
| | | TCT CC |
| J-Kappa 1a | (SEQ ID NO:84) | TT GAT CTC GAG CTT GGT |
| | | CCC TTG GCC GAA |
| J-Kappa 2a | (SEQ ID NO:85) | TT GAT CTC GAG CTT GGT |
| | | CCC CTG GCC AAA |
| J-Kappa 3a | (SEQ ID NO:86) | TT GAT CTC GAG TTT GGT |
| | | CCC AGG GCC GAA |
| J-Kappa 4a | (SEQ ID NO:87) | TT GAT CTC GAG CTT GGT |
| | | CCC TCC GCC GAA |
| J-Kappa 5a | (SEQ ID NO:88) | TT AAT CTC GAG TCG TGT |
| | | CCC TTG GCC GAA |
| V-Lambda 1a | (SEQ ID NO:89) | CAGAT GTG CAC TCC CAG |
| | | TCT GTG TTG ACG CAG |
| | | CCG CC |
| V-Lambda 2a | (SEQ ID NO:90) | CAGAT GTG CAC TCC CAG |
| | | TCT GCC CTG ACT CAG |
| | | CCT GC |
| V-Lambda 3Aa | (SEQ ID NO:91) | CAGAT GTG CAC TCC TCC |
| | | TAT GTG CTG ACT CAG |
| | | CCA CC |
| V-Lambda 3Ba | (SEQ ID NO:92) | CAGAT GTG CAC TCC TCT |
| | | TCT GAG CTG ACT CAG |
| | | GAC CC |
| V-Lambda 4a | (SEQ ID NO:93) | CAGAT GTG CAC TCC CAC |
| | | GTT ATA CTG ACT CAA |
| | | CCG CC |
| V-Lambda 5a | (SEQ ID NO:94) | CAGAT GTG CAC TCC CAG |
| | | GCT GTG CTC ACT CAG |
| | | CCG TC |
| V-Lambda 6a | (SEQ ID NO:95) | CAGAT GTG CAC TCC AAT |
| | | TTT ATG CTG ACT CAG |
| | | CCC CA |
| V-Lambda 7a | (SEQ ID NO:96) | CAGAT GTG CAC TCC CAG |
| | | GCT GTG GTG ACT CAG |
| | | GAG CC |
| J-Lambda 1a | (SEQ ID NO:97) | AC GGT AAG CTT GGT CCC |
| | | AGT TCC GAA GAC |
| J-Lambda 2/3a | (SEQ ID NO:98) | AC GGT AAG CTT GGT CCC |
| | | TCC GCC GAA TAC |

### Example 2

### Target Cell-Based Assays to Identify Bispecific Antibodies

A. Screening for Bispecific Antibodies which Bind to the BMPR Complex

Bone homeostasis is a consequence of the balance in activities between bone formation by osteoblasts and bone resorption by osteoclasts. Osteoblasts originate from mesenchymal cells through the well coordinated interaction between growth factors called Bone Morphogenetic Proteins (BMP's) and their receptors. Signaling by BMP's is achieved through binding of BMP to a heterodimeric receptor complex comprised of a Type I and a Type II component. The BMP receptors are serine/threonine-kinase receptor members of the TGF-β receptor superfamily. BMP receptors are activated when a BMP homodimer binds to two distinct domains on the Type I and Type II receptor components. Binding then results in phosphorylation of one of the receptor components by the other and signal propagation through to the nucleus where bone-specific transcription factors become activated and induce the cell to differentiate into an osteoblast.

There are multiple dimeric BMP, including BMP-2, BMP-3, BMP-4, BMP-5, BMP-6, and BMP-7. Because the BMPs are dimeric molecules, it is also possible to form heteromeric BMPs. For example, one dimer might consist of a BMP-2 subunit and a BMP-7 subunit. Given the number of BMP genes known, the possible number of combinations is large. Recombinant expression and subsequent purification of heterodimeric BMP is difficult because cells must be engineered to express similar levels of both subunits. In addition, because of the biochemical similarity between the homodimeric and heterodimeric BMP, purification is problematic. In spite of these obstacles, it has been shown that some heterodimeric BMPs have higher specific activities than their homodimeric counterparts. For example, implanting 10% of a heterodimer quantity can result in the same amount of bone formation as with the homodimer in the rat ectopic system (Israel, D. I., et al. Growth Factors 7:139-150 (1992)). Bispecific antibodies are identified by the methods described herein which mimic the ligand-binding activity of BMPs, thus reproducing the activity of heterodimeric or homodimeric BMP binding to BMP receptors, and producing a therapeutic effect, for example, facilitating bone healing.

Mouse C2C12 cells are a myoblast cell line derived from dystrophic mouse muscle. C2C12 cells differentiate rapidly upon confluence into contractile myotubes and produce characteristic muscle proteins (Yaffe, D. and O. Saxel Nature 270(5639):725-727 (1977)). Treatment with BMP-2 results in a shift in differentiation from muscle lineage to bone. BMP-2 treated C2C12 cells will form osteoblasts within a 2-4 day time period characterized by expression of bone-associated proteins alkaline phosphatase (ALPL) and osteocalcin (BGLAP) (Olson, E. N., et al., J. Cell Biol. 103(5):1799-1805 (1986); Massague, J., et al., Proc Natl Acad Sci USA. 83(21):8206-8210 (1986); Katagiri, T., et al., J Cell Biol. 127(6 Pt 1):1755-1766 (1994); Aoki, H., et al. J Cell Sci. 114(Pt 8):1483-1489 (2001)). The C2C12 in vitro model of osteoblast differentiation affords the opportunity to test the effect of bispecific antibodies on the process of differentiation in a relatively short time frame. Additionally, the model provides a very clean readout of differentiation as myotubes cells produce very little of the ALPL and BGLAP bone markers characteristic of osteoblasts.

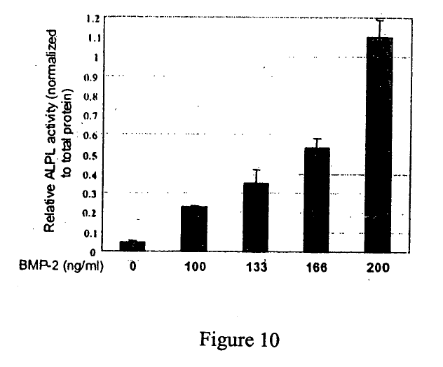

The C2C12 model of differentiation was used to replicate published results concerning BMP-2 induction of osteoblast differentiation. 1×10⁴ C2C12 cells were seeded into each well of a 24-well plate. After 24 hours (time=0), the normal growth media was removed and replaced with media containing various concentrations of BMP-2 (Peprotech, Rocky Hill, N.J.). At day 2, the conditioned media was replenished. At day 4, the media was removed and the cells were lysed by the addition of 200 ul of 10% triton. 40 ul of the lysate was added to 80 ul of 4-nitrophenyl phosphate, disodium salt hexahydrate; para-nitrophenyl phosphate (pNPP) substrate (Pierce, Rockford, Ill.). pNPP is a widely used substrate to measure the amount of ALPL in a given solution. In the presence of ALPL, pNPP produces a yellow, water-soluble solution that can be detected at 405 nm by a standard ELISA plate reader. FIG. 10 shows the average result of two independent experiments and demonstrates that quantities as low as 100 ng/ml of BMP-2 are capable of inducing C2C12 to differentiate into osteoblasts. The sensitivity of the pNPP assay is increased by any of the following three techniques; 1. increase the total amount of lysate used, 2. incubate the lysate and pNPP for a longer period of time, and 3. incubate the lysate and pNPP at 37° C.

Bispecific, monoclonal antibodies, in the context of a functional selective screen, are identified utilizing an assay similar to that described above. Specifically, bispecific antibodies secreted into the supernatant of host cells infected with vaccinia virus recombinants that encode polynucleotide libraries of the invention, along with any necessary polynucleotides encoding fixed immunoglobulin subunit polypeptides are transferred to wells seeded with C2C12 cells. It is most convenient if supernatants of infected host cells are produced in microculture plates in a 96 well format and transferred to wells seeded with C2C12 cells in a similar format. Any supernatant that includes antibodies capable of inducing differentiation of C2C12 cells into the osteoblast lineage are identified by detection of alkaline phosphatase production in the pNPP colorimetric assay read in a standard ELISA plate reader. In certain embodiments, the bispecific antibodies may have one fixed specificity for the BMP receptor type I subunit and a second undefined specificity; or one fixed specificity for BMP receptor type II subunit and a second undefined specificity; or two undefined specificities.

B. LIF Activity Assay

Leukemia Inhibitory Factor (LIF) is a member of the interleukin (IL)-6-type family of cytokines and binds to a heterodimeric receptor complex comprised of the two membrane proteins LIFRα and gp130 which also serves as receptor for oncostatin M (OSM), ciliary neurotrophic factor (CNTF), cardiotrophin-1 (CT-1), and neurotrophin-1, other members of the IL-6 family of cytokines. LIF binding to LIFRα induces heterodimerization with gp130. LIF is a pleiotropic cytokine that, among other functions, induces differentiation of mouse monocytic leukemia M1 cells, conversion of sympathetic neurons from the adrenergic to cholinergic phenotype, suppresses the differentiation of embryonic stem cells, enhances proliferation of myoblasts, and facilitates endometrial implantation of embryos. LIF is normally produced in the female reproductive tract and is naturally secreted by the endometrium during the secretory phase of embryo implantation. There is good evidence that if LIF is not secreted properly during the time of implantation, the embryo may not implant and the pregnancy may fail. Recombinant synthetic LIF is currently in clinical trials and has shown some benefit in improving embryo implantation in women with recurrent implantation failure (Serono S. A.). LIF also plays a role in the systemic inflammatory response, activating the hypothalamic-adrenal axis, and inducing the acute phase reaction of the liver. In hepatocytes, LIF, similar to other IL-6 cytokines, stimulates the enhanced expression of a set of plasma proteins, termed acute phase proteins (APP). Bispecific antibodies which bind to the LIFRα/gp130 receptor and activate the receptor complex are identified according to the methods described herein.

LIF is a pleiotropic cytokine that induces terminal differentiation and eventually apoptosis of mouse monocytic leukemia M1 cells to monocytes. CD16/32 is a marker of this differentiation and has been used to monitor the effect of LIF. Bispecific, monoclonal antibodies capable of inducing differentiation of mouse monocytic leukemia M1 cells are identified through treatment of pools of these cells in either a 24, 48, or 96-well format according to identification methods disclosed herein. Cells are then suspended and incubated with fluorescein isothiocyanate-conjugated anti-mouse CD16/32 monoclonal antibody 2.4G2 (Pharmingen) diluted at 1:200 for 30 min at 4° C. and analyzed by a FACScan with the Cell Quest program. Cell viability is determined by propidium iodide staining and a FACScan flow cytometer (Beckton Dickinson) employing methods well known to those skilled in the art.

C. GDNF Activity Assay

GDNF was purified and characterized in 1993 as a growth factor promoting the survival of the embryonic dopaminergic neurons of the midbrain, i.e. those neurons that degenerate in Parkinson's disease (Lin, L. F., et al., Science 260:1130-1132 (1993)). Soon after, it was shown that GDNF is also a very potent trophic factor for spinal motoneurons (Henderson, C. E., et al., Science. 266:1062-1064 (1994)) and central noradrenergic neurons (Arenas, E., et al., Neuron 15:1465-1473 (1995)), and therefore hopes have been raised that this growth factor may be effective as a therapeutic agent in the treatment of several neurodegenerative diseases. Recent studies have shown the additional potential of GDNF (or GDNF agonists) as therpeutic agents. For example, GDNF was recently shown to have a potential role in the regeneration of sensory axons after spinal cord injury (Ramer, M. S., et al., Nature 403:312-316 (2000)). In addition, GDNF or GDNF agonists may have the ability to regulate spermatogonia renewal and differentiation during male spermatogenesis (Meng, X., et al., Science 287:1489-1493 (2000)). Finally, an important role of GDNF in the regulation of biochemical and behavioral adaptations to chronic morphine and cocaine abuse was recently reported (Messer, C. J., et al., Neuron 26:247-257 (2000)). GDNF binds to the GDNF family receptor a (GFRα1) followed by binding of the complex to and activation of the Ret receptor tyrosine kinase (RTK). GDNF interacts with both receptor components to transduce signals through Ret. GDNF is the only member of the TGF-β superfamily known to signal through a receptor tyrosine kinase. The GFRα1/Ret signaling system is another example where activation by a bispecific antibody may function as a GDNF agonist, i.e., mimic ligand binding and facilitate activation of the receptor complex.

GDNF was initially isolated as a potent neurotrophic factor specific to the survival and differentiation of midbrain dopaminergic neurons. GDNF increases tyrosine hydroxylase (TH) expression and dopamine uptake (Lin, L. F. et al., Science 260:1130-1132 (1993)). Expression of the TH gene is upregulated by GDNF in vivo (Beck, K. D., et al. Neuron 16:665-673 (1996); Tomac, A., et al. Nature 373:335-339 (1995)) and in vitro (Theofilopoulos, S. et al., Brain Res. Dev. Brain Res. 127:111-122 (2001); Xiao, H., et al., J. Neurochem. 82:801-808 (2002)). Thus the TH gene is one of the target genes of GDNF signaling.

Very recently, an assay for GDNF signaling has been developed employing cells transfected with a DNA construct containing 2 kb of the rat TH gene promoter region driving expression of a luciferase reporter (SK-N-MC-ret/THLuc cells, Tanaka M., et al. Brain Res Brain Res Protoc. 11:119-122 (2003); Xiao, unpublished data)).

SK-N-MC-ret/THLuc cells are used to screen for bispecific antibodies which bind to GFRα1/Ret, thereby inducing GDNF-like signalling. The cells are washed with preheated (37° C.) phosphate-buffered saline (PBS) once and detached following treatment with 0.5 mM EDTA and 0.01% trypsin for 1 min. After suspension in preheated DMEM plus 10% fetal bovine serum, cells are dispensed onto a multiwell plate (0.1 ml/well on a 96-well plate) to give a cell density of 30% confluency. The multiwell plate is incubated for 24 h at 37° C. in a 5% CO₂ atmosphere to reach 60-70% confluency. After the medium is discarded and the culture is rinsed once with preheated PBS, pools of cultured media containing bispecific antibodies produced by host cells infected with vaccinia virus recombinants that encode polynucleotide libraries of the invention, along with any necessary polynucleotides encoding fixed immunoglobulin subunit polypeptides, are transferred to onto the cells, and each well is tested for the induction of luciferase activity by measuring in a conventional luminomiter.

D. Fluorogenic Caspase-Based Screen for Apoptosis-Inducing Bispecific Antibodies

A sensitive single cell-based fluorogenic assay is used for detection of apoptotic cells. Caspases are intracellular enzymes involved in the early steps of apoptosis. A cell-permeable fluorogenic caspase substrate (Liu, L. et al., Nat Med 8:185 (2002), available from Oncoimmunin, Inc.) is used to detect the presence of active caspase-6, which is a critical member of the intracellular caspase cascade. The substrate is composed of two fluorophores covalently linked to an 18-amino acid peptide containing the proteolytic cleavage sites for the individual caspase. In the uncleaved substrate, fluorescence is quenched due to the formation of intramolecular dimers, but if active caspase is present, and the substrate is cleaved, this fluorophore-fluorophore interaction is abolished, leading to an increase in fluorescence. This fluorescence can be analyzed via standard flow cytometry, or for higher throughput, using fluorometric microvolume assay technology (FMAT™). The FMAT™ system uses a 633-nm helium-neon red laser as an excitation source and can detect fluorescence from 650 to 720 nm, which is appropriate for use with the caspase substrate.

Bispecific antibodies secreted into the supernatant of host cells infected with vaccinia virus recombinants that encode polynucleotide libraries of the invention, along with any necessary polynucleotides encoding fixed immunoglobulin subunit polypeptides, are transferred to wells containing viable tumor cells. It is most convenient if supernatants of infected host cells are produced in microculture plates in a 96 well format and transferred to wells seeded with target tumor cells in a similar format. Multiple tumor cell lines of diverse tissue origin may be utilized including, for example, HeLa, a human cervical adenocarcinoma carcinoma cell line, Jurkat, a T cell leukemia cell line, and 21NT, a human breast cancer cell line. After incubation for 1-2 hours at 37° C. with sample supernatants, cells are incubated with the caspase substrate for 30 minutes, and subsequently analyzed on the FMAT™ system. Jurkat cells are used as a positive control by using an anti-Fas (CD95/APO-1) antibody, clone 2R2 (Oehm, A. et al., J Biol Chem 267:10709 (1992)) that has been shown to induce apoptosis. Negative controls include adding supernatant to cells without adding the caspase substrate, or adding non-antibody containing supernatant from wild-type vaccinia-infected cells to cells with substrate. Assay wells with an increase in fluorescence above background indicate the presence of active caspase enzyme, and therefore the presence of a bispecific antibody that can induce apoptosis. Vaccinia-encoded antibody from positive wells on the corresponding minilibrary plates are then divided into subpools, expanded out and rescreened using either the FMAT™ system or standard flow cytometry on a FACScalibur (BD Biosciences).

### Example 3

### Selection of an Antibody with Defined Specificity from a Library of 10⁹ Combinations of Immunoglobulin Heavy and Light Chains

This example is directed to defining the specific methods for producing and identifying monospecific bivalent antibodies. Based on disclosures elsewhere in this application, one of ordinary skill in the art could readily apply these methods to produce and identify bispecific antibodies, either bivalent or tetravalent. The affinity of specific antibodies that can be selected from a library is a function of the size of that library. In general, the larger the number of heavy and light chain combinations represented in the library, the greater the likelihood that a high affinity bispecific antibody is present and can be selected. Previous work employing phage display methods has suggested that for many antigens a library that includes 10⁹ immunoglobulin heavy and light chain combinations is of a sufficient size to select a relatively high affinity specific antibody. In principle, it is possible to construct a library with 10⁹ recombinants each of which expresses a unique heavy chain and a unique light chain or a single chain construct with a combining site comprising variable regions of heavy and light chains. The most preferred method, however, is to generate this number of antibody combinations by constructing two or more libraries of 10⁵ immunoglobulin heavy chains (with various heterodimerization domains and/or means for tetramerization) and 10⁴ immunoglobulin light chains that can be co-expressed in all 10⁹ (or 10¹⁴ for three libraries) possible combinations. In this example greater diversity is represented in the heavy chain pool(s) because heavy chains have often been found to make a greater contribution than the associated light chain to a specific antigen combining site.

3.1 Heavy Chain Genes. One or more libraries of vaccinia recombinants at a titer of approximately 10 is constructed from a minimum of 1 immunoglobulin heavy chain cDNA transfer plasmid recombinants synthesized by the methods previously described (Example 1) from RNA derived from a pool of 100 bone marrow donors. As described below, this library must be further expanded to a titer of at least 10⁹ heavy chain recombinants. A preferred method to expand the library is to infect microcultures of approximately 5×10⁴ BSC1 cells with individual pools of 10³ vaccinia heavy chain recombinants. Typically a greater than 1,000 fold expansion in the viral titer is obtained after 48 hrs infection. Expanding viral titers in multiple individual pools mitigates the risk that a subset of recombinants will be lost due to relatively rapid growth of a competing subset.

3.2 Light Chain Genes. A library of vaccinia recombinants at a titer of approximately 10⁵ is constructed from a minimum of 10⁴ immunoglobulin light chain cDNA transfer plasmid recombinants synthesized from RNA derived from a pool of bone marrow donors as described in Example 1. For use in multiple cycles of heavy chain selection as described below, this library must be further expanded to a titer of 10¹⁰ to 10¹¹ light chain recombinants. A preferred method to expand the library is to infect 100 microcultures of approximately 5×10⁴ BSC1 cells with individual pools of 10³ vaccinia light chain recombinants. Viral recombinants recovered from each of the 100 infected cultures are further expanded as a separate pool to a titer of between 10⁸ and 10⁹ viral recombinants. It is convenient to label these light chain pools L1 to L100. While this example illustrates identification of monospecific bivalent immunoglobulin molecules, similar procedures are used to identify bispecific bivalent or bispecific tetravalent antibodies of the present invention.

3.3 Identification of Immunoglobulin Heavy Chain Recombinants. 100 cultures of 10⁷ cells of a non-producing myeloma, preferably Sp2/0, or early B cell lymphoma, preferably CH33, are infected with viable vaccinia heavy chain recombinants at MOI=1 to 10 and simultaneously with psoralen (4′-aminomethyl-Trioxsalen) inactivated vaccinia light chain recombinants at MOI=1 to 10 (see below). For psoralen inactivation, cell-free virus at 10⁸ to 10⁹ pfu/ml is treated with 10 μg/ml psoralen for 10 minutes at 25° C. and then exposed to long-wave (365-nm) UV light for 2 minutes (Tsung, K., J. H. Yim, W. Marti, R. M. L. Buller, and J. A. Norton. J. Virol. 70:165-171 (1996)). The psoralen treated virus is unable to replicate but allows expression of early viral genes including recombinant genes under the control of early but not late viral promoters. Under these conditions, light chains synthesized from psoralen treated recombinants will be assembled into immunoglobulin molecules in association with the single heavy chain that is, on average, expressed in each infected cell.

The choice of infection with psoralen inactivated light chain recombinants at MOI=1 or at MOI=10 will influence the relative concentration in a single positive cell of particular H+L chain combinations which will be high at MOI=1 and low (because of dilution by multiple light chains) at MOI=10. A low concentration and correspondingly reduced density of specific immunoglobulin at the cell surface is expected to select for antibodies with higher affinity for the ligand of interest. On the other hand, a high concentration of specific receptor is expected to facilitate binding or signaling through the immunoglobulin receptor.

Following a first identification step by any of the methods described herein, an enriched population of recombinant virus is recovered from each culture with a titer which, during this initial selection and depending on background levels of non-specific binding or spontaneous release of virus, may be between 1% and 10% of the titer of input virus. It is convenient to label as H1a to H100a the heavy chain recombinant pools recovered from cultures in the first cycle of selection that received psoralen treated virus from the original light chain recombinant pools L1 to L100 respectively.

To carry out a second round of enrichment under the same conditions as the first cycle, it is again necessary to expand the titer of recovered heavy chain recombinants by 10 to 100 fold. For the second cycle of selection non-producing myeloma or early B cell lymphoma are again infected with viable viral heavy chain recombinants and psoralen treated light chain recombinants such that, for example, the same culture of 10⁷ cells is infected with heavy chain recombinants recovered in pool H37a and psoralen treated light chain recombinants from the original L37 pool employed to select H37a. Heavy chain recombinants recovered from the H37a pool in the second cycle of selection are conveniently labeled H37b and so on.

Following the second round of enrichment, specific viral recombinants are likely, in general, to be enriched by a factor of 10 or more relative to the initial virus population. In this case, it is not necessary for the third cycle of enrichment to be carried out under the same conditions as the first or second cycle since specific clones are likely to be well-represented even at a 10 fold lower titer. For the third cycle of enrichment, therefore, 100 cultures of only 10⁶ non-producing myeloma or early B cell lymphoma are again infected with viable viral heavy chain recombinants and psoralen treated light chain recombinants from cognate pools. Another reduction by a factor of 10 in the number of infected cells is effected after the 5th cycle of selection.

3.4 Identification of Antigen-specific Heavy Chain Recombinants. (a) Following any given cycle of enrichment it is possible to determine whether antigen-specific heavy chains have been enriched to a level of 10% or more in a particular pool, for example H37f, by picking 10 individual viral pfu from that heavy chain pool to test for antigen-specificity in association with light chains of the original L37 pool. Since the light chain population comprises 104 diverse cDNA distributed among 100 individual pools, the average pool has approximately 102 different light chains. Even if a selected heavy chain confers a desired antigenic specificity only in association with a single type of light chain in the available light chain pool, 1% of cells infected with the selected heavy chain recombinant and the random light chain pool at MOI=1 will express the desired specificity. This frequency can be increased to 10% on average if cells are infected with light chains at MOI=10. A preferred method to confirm specificity is to infect with immunoglobulin heavy chain and a pool of light chains a line of CH33 early B cell lymphoma transfected with an easily detected reporter construct, for example luciferase, driven by the promoter for BAX or another CH33 gene that is activated as a result of membrane receptor crosslinking. Infection of this transfectant with the plaque purified heavy chain recombinant and the relevant light chain pool will result in an easily detected signal if the selected heavy chain confers the desired antigenic specificity in association with any of the 100 or more light chains represented in that pool. Note that this same method is applicable to analysis of heavy chains whether they are selected by specific-binding or by specific-signaling through immunoglobulin receptors of infected cells.

(b) An alternative method to identify the most promising antigen-specific heavy chains is to screen for those that are most highly represented in the enriched population. Inserts can be isolated by PCR amplification with vector specific primers flanking the insertion site and these inserts can be sequenced to determine the frequency of any observed sequence. In this case, however, it remains necessary to identify a relevant light chain as described below.

3.5 Identification of Immunoglobulin Light Chain Recombinants. Once an antigen-specific heavy chain has been isolated, a light chain that confers antigen-specificity in association with that heavy chain can be isolated from the pool that was employed to select that heavy chain as described in 3.4(a). Alternatively, it may be possible to select yet another light chain from a larger library that, in association with the same heavy chain, could further enhance affinity. For this purpose a library of vaccinia recombinants at a titer of approximately 10⁶ is constructed from a minimum of 10⁵ immunoglobulin light chain cDNA transfer plasmid recombinants synthesized by the methods previously described (Example 1). The procedure described in 3.3 is reversed such that non-producing myeloma or early B cell lymphoma are now infected with viable viral light chain recombinants at MOI=1 and a single selected psoralen treated specific heavy chain recombinant. To promote selection of higher affinity immunoglobulin, it may be preferable to dilute the concentration of each specific H+L chain pair by infection with light chains at MOI=10.

3.6 Identification of Immunoglobulin Heavy Chain Recombinants in the Presence of a Single Immunoglobulin Light Chain. The identification of an immunoglobulin heavy chain that can contribute to a particular antibody specificity is simplified if a candidate light chain has already been identified. This may be the case if, for example a murine monoclonal antibody has been previously selected. The murine light chain variable region can be grafted to a human light chain constant region to optimize pairing with human heavy chains, a process previously described by others employing phage display methods as “Guided Selection” (Jespers, L. S., et al., Bio/Technology 12:899-903, 1994; Figini, M., et al., Cancer Res. 58:991-996, 1998). This molecular matching can, in principle, be taken even further if human variable gene framework regions are also grafted into the murine light chain variable region sequence (Rader, C., et al., Proc. Natl. Acad. Sci. USA 95:8910-8915). Any human heavy chains selected to pair with this modified antigen-specific light chain can themselves become the basis for selection of an optimal human light chain from a more diverse pool as described in 3.5.

### Example 4

### Identification of Monospecific Human Antibodies from a cDNA Library Constructed in Adenovirus, Herpesvirus, or Retrovirus Vectors

This example describes the identification of monospecific bivalent antibodies from libraries constructed in three different animal viruses. Based on disclosures elsewhere in this application, one of ordinary skill in the art could readily apply these methods to the identification of bispecific antibodies either bivalent or tetravalent.

4.1 Herpesvirus. A method has been described for the generation of helper virus free stocks of recombinant, infectious herpes simplex virus amplicons (T. A. Stavropoulos, and C. A. Strathdee, J. Virology 72:7137-7143 (1998)). It is possible that a cDNA library of human immunoglobulin heavy and/or light chain genes or fragments thereof, including single chain fragments, constructed in the plasmid amplicon vector could be packaged into a library of infectious amplicon particles using this method. An amplicon library constructed using immunoglobulin heavy chain genes, and another amplicon library constructed using immunoglobulin light chain genes could be used to coinfect a non-producing myeloma cell line. The myeloma cells expressing an immunoglobulin gene combination with the desired specificity can be enriched by selection for binding to the antigen of interest. The herpes amplicons are capable of stable transgene expression in infected cells. Cells selected for binding in a first cycle will retain their immunoglobulin gene combination, and will stably express antibody with this specificity. This allows for the reiteration of selection cycles until immunoglobulin genes with the desired specificity can be isolated. Selection strategies that result in cell death could also be attempted. The amplicon vector recovered from these dead selected cells cannot be used to infect fresh target cells, because in the absence of helper virus the amplicons are replication defective and will not be packaged into infectious form. The amplicon vectors contain a plasmid origin of replication and an antibiotic resistance gene. This makes it possible to recover the selected amplicon vector by transforming DNA purified from the selected cells into bacteria. Selection with the appropriate antibiotic would allow for the isolation of bacterial cells that had been transformed by the amplicon vector. The use of different antibiotic resistance genes on the heavy and light chain amplicon vectors, for example ampicillin and kanamycin, would allow for the separate selection of heavy and light chain genes from the same population of selected cells. Amplicon plasmid DNA can be extracted from the bacteria and packaged into infectious viral particles by cotransfection of the amplicon DNA and packaging defective HSV genomic DNA into packaging cells. Infectious amplicon particles can then be harvested and used to infect a fresh population of target cells for another round of selection

4.2 Adenovirus. Methods have been described for the production of recombinant Adenovirus (S. Miyake, et al., Proc. Natl. Acad. Sci. USA 93: 1320-1324 (1996); T. C. He, et al., Proc. Natl. Acad. Sci. USA 95: 2509-2514 (1998) It is possible that a cDNA library could be constructed in an Adenovirus vector using either of these methods. Insertion of cDNA into the E3 or E4 region of Adenovirus results in a replication competent recombinant virus. This library could be used for similar applications as the vaccinia cDNA libraries constructed by trimolecular recombination. For example a heavy chain cDNA library can be inserted into the E3 or E4 region of adenovirus. This results in a replication competent heavy chain library. A light chain cDNA library could be inserted into the E1 gene of Adenovirus, generating a replication defective library. This replication defective light chain library can be amplified by infection of cells that provide Adenovirus E1 in trans, such as 293 cells. These two libraries can be used in similar selection strategies as those described using replication competent vaccinia heavy chain library and Psoralen inactivated vaccinia light chain library.

4.3 Advantages of vaccinia virus. Vaccinia virus possesses several advantages over herpes or adenovirus for construction of cDNA libraries. First, vaccinia virus replicates in the cytoplasm of the host cell, while HSV and adenovirus replicate in the nucleus. A higher frequency of cDNA recombinant transfer plasmid may be available for recombination in the cytoplasm with vaccinia than is able to translocate into the nucleus for packaging/recombination in HSV or adenovirus. Second, vaccinia virus, but not adenovirus or herpes virus, is able to replicate plasmids in a sequence independent manner (M. Merchlinsky, and B. Moss., Cancer Cells 6: 87-93 (1988)). Vaccinia replication of cDNA recombinant transfer plasmids may result in a higher frequency of recombinant virus being produced.

4.4 Retrovirus. Construction of cDNA Libraries in replication defective retroviral vectors have been described (T. Kitamura, et al., PNAS 92:9146-9150 (1995); I. Whitehead, et al., Molecular and Cellular Biology 15:704-710 (1995)). Retroviral vectors integrate upon infection of target cells, and have gained widespread use for their ability to efficiently transduce target cells, and for their ability to induce stable transgene expression. A retroviral cDNA library constructed using immunoglobulin heavy chain genes, and another retroviral library constructed using immunoglobulin light chain genes could be used to coinfect a non-producing myeloma cell line. The myeloma cells expressing an immunoglobulin gene combination with the desired specificity can be enriched for by selection for binding to the antigen of interest. Cells selected for binding in a first cycle will retain their immunoglobulin gene combination, and will stably express immunoglobulins with this specificity. This allows for the reiteration of selection cycles until immunoglobulin genes with the desired specificity can be isolated.

### Example 5

### Trimolecular Recombination

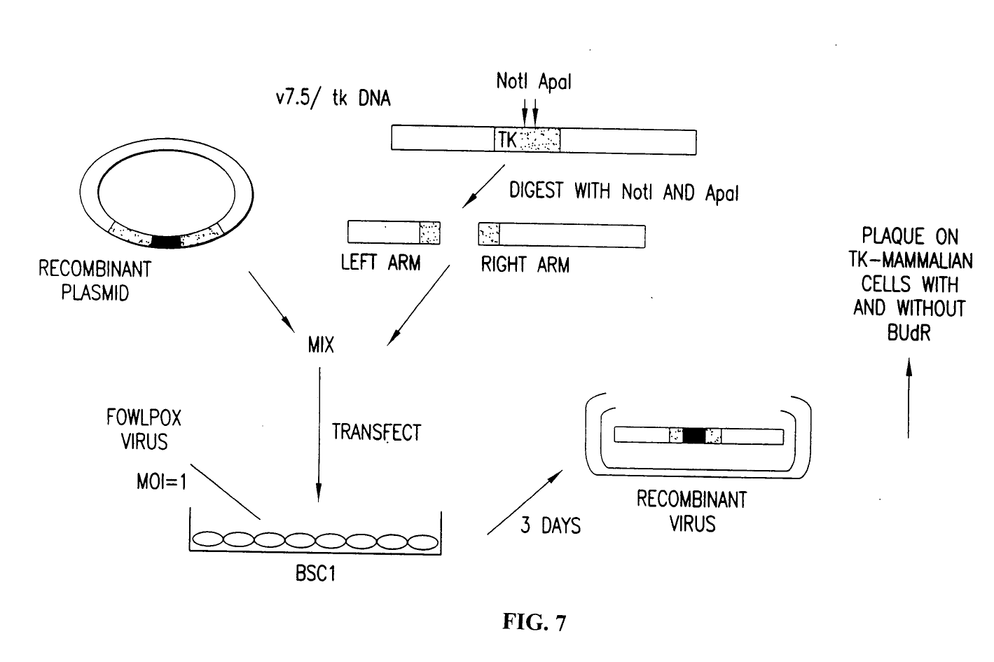

5.1 Production of an Expression Library. This example describes a tri-molecular recombination method employing modified vaccinia virus vectors and related transfer plasmids that generates close to 100% recombinant vaccinia virus and, for the first time, allows efficient construction of a representative DNA library in vaccinia virus. The trimolecular recombination method is illustrated in FIG. 7.

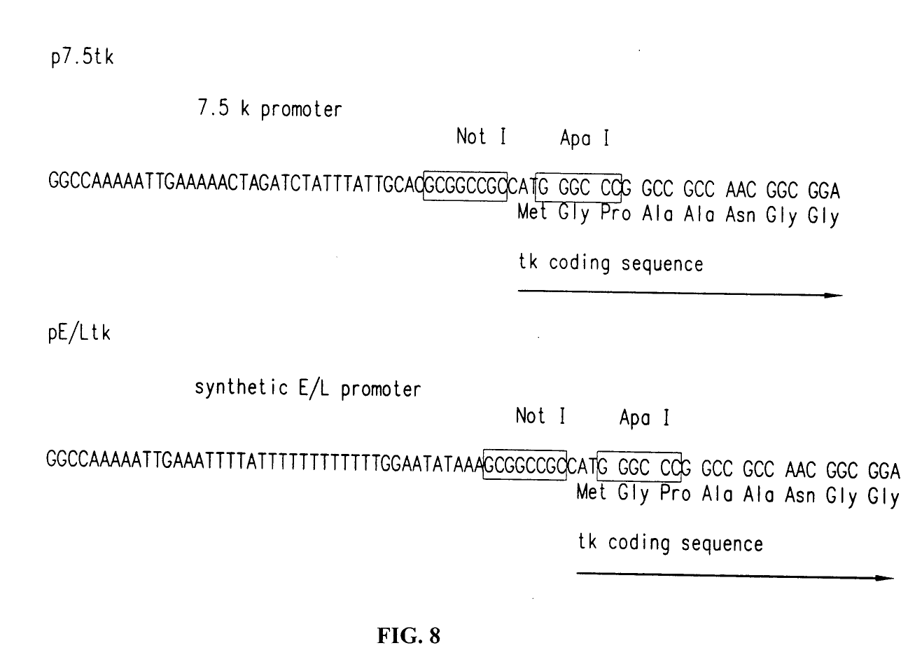

5.2 Construction of the Vectors. The previously described vaccinia virus transfer plasmid pJ/K, a pUC 13 derived plasmid with a vaccinia virus thymidine kinase gene containing an in-frame Not I site (Merchlinsky, M. et al., Virology 190:522-526), was further modified to incorporate a strong vaccinia virus promoter followed by Not I and Apa I restriction sites. Two different vectors, p7.5/tk and pEL/tk, included, respectively, either the 7.5K vaccinia virus promoter or a strong synthetic early/late (E/L) promoter (FIG. 8). The Apa I site was preceded by a strong translational initiation sequence including the ATG codon. This modification was introduced within the vaccinia virus thymidine kinase (tk) gene so that it was flanked by regulatory and coding sequences of the viral tk gene. The modifications within the tk gene of these two new plasmid vectors were transferred by homologous recombination in the flanking tk sequences into the genome of the Vaccinia Virus WR strain derived vNotI⁻ vector to generate new viral vectors v7.5/tk and vEL/tk. Importantly, following Not I and Apa I restriction endonuclease digestion of these viral vectors, two large viral DNA fragments were isolated each including a separate non-homologous segment of the vaccinia tk gene and together comprising all the genes required for assembly of infectious viral particles. Further details regarding the construction and characterization of these vectors and their alternative use for direct ligation of DNA fragments in vaccinia virus are described in Example 1.

5.3 Generation of an Increased Frequency of Vaccinia Virus Recombinants. Standard methods for generation of recombinants in vaccinia virus exploit homologous recombination between a recombinant vaccinia transfer plasmid and the viral genome. Table 3 shows the results of a model experiment in which the frequency of homologous recombination following transfection of a recombinant transfer plasmid into vaccinia virus infected cells was assayed under standard conditions. To facilitate functional assays, a minigene encoding the immunodominant 257-264 peptide epitope of ovalbumin in association with H-2 K^{b} was inserted at the Not 1 site in the transfer plasmid tk gene. As a result of homologous recombination, the disrupted tk gene is substituted for the wild type viral tk+ gene in any recombinant virus. This serves as a marker for recombination since tk− human 143B cells infected with tk− virus are, in contrast to cells infected with wild type tk+ virus, resistant to the toxic effect of BrdU. Recombinant virus can be scored by the viral pfu on 143B cells cultured in the presence of 125 mM BrdU.

The frequency of recombinants derived in this fashion is of the order of 0.1% (Table 3).

TABLE 3
Generation of Recombinant Vaccinia Virus
by Standard Homologous Recombination

| | | Titer w/o | Titer | % |
|---|---|---|---|---|
| Virus* | DNA | BrdU | w/BrdU | Recombinant** |
| vaccinia | — | 4.6 × 10⁷ | 3.0 × 10³ | 0.006 |
| vaccinia | 30 ng pE/Lova | 3.7 × 10⁷ | 3.2 × 10⁴ | 0.086 |
| vaccinia | 300 ng pE/Lova | 2.7 × 10⁷ | 1.5 × 10⁴ | 0.056 |

*vaccinia virus strain vNotI
**% Recombinant = (Titer with BrdU/Titer without BrdU) × 100

This recombination frequency is too low to permit efficient construction of a cDNA library in a vaccinia vector. The following two procedures were used to generate an increased frequency of vaccinia virus recombinants.

(1) One factor limiting the frequency of viral recombinants generated by homologous recombination following transfection of a plasmid transfer vector into vaccinia virus infected cells is that viral infection is highly efficient whereas plasmid DNA transfection is relatively inefficient. As a result many infected cells do not take up recombinant plasmids and are, therefore, capable of producing only wild type virus. In order to reduce this dilution of recombinant efficiency, a mixture of naked viral DNA and recombinant plasmid DNA was transfected into Fowl Pox Virus (FPV) infected mammalian cells. As previously described by others (Scheiflinger, F., et al., Proc. Natl. Acad. Sci. USA 89:9977-9981 (1992)), FPV does not replicate in mammalian cells but provides necessary helper functions required for packaging mature vaccinia virus particles in cells transfected with non-infectious naked vaccinia DNA. This modification of the homologous recombination technique alone increased the frequency of viral recombinants approximately 35 fold to 3.5% (Table 4).

TABLE 4
Generation of Recombinant Vaccinia Virus
by Modified Homologous Recombination

| | | Titer w/o | Titer | % |
|---|---|---|---|---|
| Virus | DNA | BrdU | w/BrdU | Recombinant* |
| PFV | None | 0 | 0 | 0 |
| None | vaccinia WR | 0 | 0 | 0 |
| PFV | vaccinia WR | 8.9 × 10⁶ | 2.0 × 10² | 0.002 |
| PFV | vaccinia WR + | 5.3 × 10⁶ | 1.2 × 10⁵ | 2.264 |
| | pE/Lova (1:1) | | | |
| PFV | vaccinia WR + | 8.4 × 10⁵ | 3.0 × 10⁴ | 3.571 |
| | pE/Lova (1:10) | | | |

*% Recombinant = (Titer with BrdU/Titer without BrdU) × 100

Table 4. Confluent monolayers of BSC1 cells (5×10⁵ cells/well) were infected with moi=1.0 of fowlpox virus strain HP1. Two hours later supernatant was removed, cells were washed 2× with Opti-Mem I media, and transfected using lipofectamine with 600 ng vaccinia strain WR genomic DNA either alone, or with 1:1 or 1:10 (vaccinia:plasmid) molar ratios of plasmid pE/Lova. This plasmid contains a fragment of the ovalbumin cDNA, which encodes the SIINFEKL epitope (SEQ. ID NO:99), known to bind with high affinity to the mouse class I MHC molecule Kb. Expression of this minigene is controlled by a strong, synthetic Early/Late vaccinia promoter. This insert is flanked by vaccinia tk DNA. Three days later cells were harvested, and virus extracted by three cycles of freeze/thaw in dry ice isopropanol/37° C. water bath. Crude virus stocks were titered by plaque assay on human TK− 143B cells with and without BrdU.

(2) A further significant increase in the frequency of viral recombinants was obtained by transfection of FPV infected cells with a mixture of recombinant plasmids and the two large approximately 80 kilobases and 100 kilobases fragments of vaccinia virus v7.5/tk DNA produced by digestion with Not I and Apa I restriction endonucleases. Because the Not I and Apa I sites have been introduced into the tk gene, each of these large vaccinia DNA arms includes a fragment of the tk gene. Since there is no homology between the two tk gene fragments, the only way the two vaccinia arms can be linked is by bridging through the homologous tk sequences that flank the inserts in the recombinant transfer plasmid. The results in Table 5 show that >99% of infectious vaccinia virus produced in triply transfected cells is recombinant for a DNA insert as determined by BrdU resistance of infected tk− cells.

TABLE 5
Generation of 100% Recombinant Vaccinia
Virus Using Tri-Molecular Recombination

| | | Titer w/o | Titer | % |
|---|---|---|---|---|
| Virus | DNA | BrdU | w/BrdU | Recombinant* |
| PFV | Uncut v7.5/tk | 2.5 × 10⁶ | 6.0 × 10³ | 0.24 |
| PFV | NotI/Apal | 2.0 × 10² | 0 | 0 |
| | v7.5/tk arms | | | |
| PFV | NotI/Apal | 6.8 × 10⁴ | 7.4 × 10⁴ | 100 |
| | v7.5/tk arms + | | | |
| | pE/Lova (1:1) | | | |

*% Recombinant = (Titer with BrdU/Titer without BrdU) × 100

Table 5. Genomic DNA from vaccinia strain V7.5/tk (1.2 micrograms) was digested with ApaI and NotI restriction endonucleases. The digested DNA was divided in half. One of the pools was mixed with a 1:1 (vaccinia:plasmid) molar ratio of pE/Lova. This plasmid contains a fragment of the ovalbumin cDNA, which encodes the SIINFEKL (SEQ ID NO:99) epitope, known to bind with high affinity to the mouse class I MHC molecule Kb. Expression of this minigene is controlled by a strong, synthetic Early/Late vaccinia promoter. This insert is flanked by vaccinia tk DNA. DNA was transfected using lipofectamine into confluent monolayers (5×10⁵ cells/well) of BSC1 cells, which had been infected 2 hours previously with moi=1.0 FPV. One sample was transfected with 600 ng untreated genomic V7.5/tk DNA. Three days later cells were harvested, and the virus was extracted by three cycles of freeze/thaw in dry ice isopropanol/37° C. water bath. Crude viral stocks were plaqued on TK-143 B cells with and without BrdU selection.

5.4 Construction of a Representative cDNA Library in Vaccinia Virus. A cDNA library is constructed in the vaccinia vector to demonstrate representative expression of known cellular mRNA sequences. Additional modifications have been introduced into the p7.5/tk transfer plasmid and v7.5/tk viral vector to enhance the efficiency of recombinant expression in infected cells. These include introduction of translation initiation sites in three different reading frames and of both translational and transcriptional stop signals as well as additional restriction sites for DNA insertion.

First, the HindIII J fragment (vaccinia tk gene) of p7.5/tk was subcloned from this plasmid into the HindIII site of pBS phagemid (Stratagene) creating pBS.Vtk.

Second, a portion of the original multiple cloning site of pBS.Vtk was removed by digesting the plasmid with SmaI and PstI, treating with Mung Bean Nuclease, and ligating back to itself, generating pBS.Vtk.MCS−. This treatment removed the unique SmaI, BamHI, SalI, and PstI sites from pBS.Vtk.

Third, the object at this point was to introduce a new multiple cloning site downstream of the 7.5k promoter in pBS.Vtk.MCS−. The new multiple cloning site was generated by PCR using 4 different upstream primers, and a common downstream primer. Together, these 4 PCR products would contain either no ATG start codon, or an ATG start codon in each of the three possible reading frames. In addition, each PCR product contains at its 3 prime end, translation stop codons in all three reading frames, and a vaccinia virus transcription double stop signal. These 4 PCR products were ligated separately into the NotI/ApaI sites of pBS.Vtk.MCS-, generating the 4 vectors, p7.5/ATG0/tk, p7.5/ATG1/tk, p7.5/ATG2/tk, and p7.5/ATG3/tk (produced as described in example 1 of U.S. Patent Application Publication No. U.S. Pat. No. 2,002,0018785A1, published Sep. 5, 2002 and PCT Publication WO 02102855, published Dec. 27, 2002, each incorporated herein by reference). Each vector includes unique BamHI, SmaI, PstI, and SalI sites for cloning DNA inserts that employ either their own endogenous translation initiation site (in vector p7.5/ATG0/tk) or make use of a vector translation initiation site in any one of the three possible reading frames (p7.5/ATG1/tk, p7.5/ATG3/tk, and p7.5/ATG4/tk).

In a model experiment cDNA was synthesized from poly-A+ mRNA of a murine tumor cell line (BCA39) and ligated into each of the four modified p7.5/tk transfer plasmids. The transfer plasmid is amplified by passage through procaryotic host cells such as E. coli as described herein or as otherwise known in the art. Twenty micrograms of Not I and Apa I digested v/tk vaccinia virus DNA arms and an equimolar mixture of the four recombinant plasmid cDNA libraries was transfected into FPV helper virus infected BSC-1 cells for tri-molecular recombination. The virus harvested had a total titer of 6×10⁶ pfu of which greater than 90% were BrdU resistant.

In order to characterize the size distribution of cDNA inserts in the recombinant vaccinia library, individual isolated plaques were picked using a sterile pasteur pipette and transferred to 1.5 ml tubes containing 100 μl Phosphate Buffered Saline (PBS). Virus was released from the cells by three cycles of freeze/thaw in dry ice/isopropanol and in a 37° C. water bath. Approximately one third of each virus plaque was used to infect one well of a 12 well plate containing tk− human 143B cells in 250 μl final volume. At the end of the two hour infection period each well was overlayed with 1 ml DMEM with 2.5% fetal bovine serum (DMEM-2.5) and with BUdR sufficient to bring the final concentration to 125 μg/ml. Cells were incubated in a CO₂ incubator at 37° C. for three days. On the third day the cells were harvested, pelleted by centrifugation, and resuspended in 500 μl PBS. Virus was released from the cells by three cycles of freeze/thaw as described above. Twenty percent of each virus stock was used to infect a confluent monolayer of BSC-1 cells in a 50 mm tissue culture dish in a final volume of 3 ml DMEM-2.5. At the end of the two hour infection period the cells were overlayed with 3 ml of DMEM-2.5. Cells were incubated in a CO₂ incubator at 37° C. for three days. On the third day the cells were harvested, pelleted by centrifugation, and resuspended in 300 μl PBS. Virus was released from the cells by three cycles of freeze/thaw as described above. One hundred microliters of crude virus stock was transferred to a 1.5 ml tube, an equal volume of melted 2% low melting point agarose was added, and the virus/agarose mixture was transferred into a pulsed field gel sample block. When the agar worms were solidified they were removed from the sample block and cut into three equal sections. All three sections were transferred to the same 1.5 ml tube, and 250 μl of 0.5M EDTA, 1% Sarkosyl, 0.5 mg/ml Proteinase K was added. The worms were incubated in this solution at 37° C. for 24 hours. The worms were washed several times in 500 μl 0.5×TBE buffer, and one section of each worm was transferred to a well of a 1% low melting point agarose gel. After the worms were added the wells were sealed by adding additional melted 1% low melting point agarose. This gel was then electorphoresed in a Bio-Rad pulsed field gel electrophoresis apparatus at 200 volts, 8 second pulse times, in 0.5×TBE for 16 hours. The gel was stained in ethidium bromide, and portions of agarose containing vaccinia genomic DNA were excised from the gel and transferred to a 1.5 ml tube. Vaccinia DNA was purified from the agarose using β-Agarase (Gibco) following the recommendations of the manufacturer. Purified vaccinia DNA was resuspended in 50 μl ddH₂O. One microliter of each DNA stock was used as the template for a Polymerase Chain Reaction (PCR) using vaccinia TK specific primers MM428 and MM430 (which flank the site of insertion) and Klentaq Polymerase (Clontech) following the recommendations of the manufacturer in a 20 μl final volume. Reaction conditions included an initial denaturation step at 95° C. for 5 minutes, followed by 30 cycles of: 94° C. 30 seconds, 55° C. 30 seconds, 68° C. 3 minutes. Two and a half microliters of each PCR reaction was resolved on a 1% agarose gel, and stained with ethidium bromide. Amplified fragments of diverse sizes were observed. When corrected for flanking vector sequences amplified in PCR the inserts range in size between 300 and 2500 bp.

Representative expression of gene products in this library was established by demonstrating that the frequency of specific cDNA recombinants in the vaccinia library was indistinguishable from the frequency with which recombinants of the same cDNA occur in a standard plasmid library. This is illustrated in Table 6 for an IAP sequence that was previously shown to be upregulated in murine tumors. Twenty separate pools with an average of either 800 or 200 viral pfu from the vaccinia library were amplified by infecting microcultures of 143B tk-cells in the presence of BDUR. DNA was extracted from each infected culture after three days and assayed by PCR with sequence specific primers for the presence of a previously characterized endogenous retrovirus (IAP, intracisternal A particle) sequence. Poisson analysis of the frequency of positive pools indicates a frequency of one IAP recombinant for approximately every 500 viral pfu (Table 6). Similarly, twenty separate pools with an average of either 1,400 or 275 bacterial cfu from the plasmid library were amplified by transformation of DH5 a bacteria. Plasmid DNA from each pool was assayed for the presence of the same IAP sequence. Poisson analysis of the frequency of positive pools indicates a frequency of one LAP recombinant for every 450 plasmids (Table 6).

TABLE 6
Limiting dilution analysis of IAP sequences in a recombinant
Vaccinia library and a conventional plasmid cDNA library

| | # Wells | | | |
|---|---|---|---|---|
| | Positiveby | | | |
| | PCR | F₀ | μ | Frequency |
| #PFU/well | | Vaccinia Library | | |
| 800 | 18/20 | 0.05 | 2.3 | 1/350 |
| 200 | 6/20 | 0.7 | 0.36 | 1/560 |
| #CFU/well | | Plasmid Library | | |
| 1400 | 20/20 | 0 | — | — |
| 275 | 9/20 | 0.55 | 0.6 | 1/450 |

F₀ = fraction negative wells;
μ = DNA precursors/well = −lnF₀

Similar analysis was carried out with similar results for representation of an alpha tubulin sequence in the vaccinia library. The comparable frequency of arbitrarily chosen sequences in the two libraries constructed from the same tumor cDNA suggests that although construction of the Vaccinia library is somewhat more complex and is certainly less conventional than construction of a plasmid library, it is equally representative of tumor cDNA sequences.

Discussion

The above-described tri-molecular recombination strategy yields close to 100% viral recombinants. This is a highly significant improvement over current methods for generating viral recombinants by transfection of a plasmid transfer vector into vaccinia virus infected cells. This latter procedure yields viral recombinants at a frequency of the order of only 0.1%. The high yield of viral recombinants in tri-molecular recombination makes it possible, for the first time, to efficiently construct genomic or cDNA libraries in a vaccinia virus derived vector. In the first series of experiments a titer of 6×10⁶ recombinant virus was obtained following transfection with a mix of 20 micrograms of Not I and Apa I digested vaccinia vector arms together with an equimolar concentration of tumor cell cDNA. This technological advance creates the possibility of new and efficient screening and selection strategies for isolation of specific genomic and cDNA clones.

The tri-molecular recombination method as herein disclosed may be used with other viruses such as mammalian viruses including vaccinia and herpes viruses. Typically, two viral arms which have no homology are produced. The only way that the viral arms can be linked is by bridging through homologous sequences that flank the insert in a transfer vector such as a plasmid. When the two viral arms and the transfer vector are present in the same cell the only infectious virus produced is recombinant for a DNA insert in the transfer vector.

Libraries constructed in vaccinia and other mammalian viruses by the tri-molecular recombination method of the present invention may have similar advantages to those described here for vaccinia virus and its use in identifying target antigens in the CTL screening system of the invention. Similar advantages are expected for DNA libraries constructed in vaccinia or other mammalian viruses when carrying out more complex assays in eukaryotic cells. Such assays include but are not limited to screening for DNA encoding receptors and ligands of eukaryotic cells.

### Example 6

### Preparation of Transfer Plasmids

The transfer vectors may be prepared for cloning by known means. A preferred method involves cutting 1-5 micrograms of vector with the appropriate restriction endonucleases (for example SmaI and SalI or BamHI and SalI) in the appropriate buffers, at the appropriate temperatures for at least 2 hours. Linear digested vector is isolated by electrophoresis of the digested vector through a 0.8% agarose gel. The linear plasmid is excised from the gel and purified from agarose using methods that are well known.

Ligation. The cDNA and digested transfer vector are ligated together using well known methods. In a preferred method 50-100 ng of transfer vector is ligated with varying concentrations of cDNA using T4 DNA Ligase, using the appropriate buffer, at 14° C. for 18 to 24 hours.

Transformation. Aliquots of the ligation reactions are transformed by electroporation into E. Coli bacteria such as DH10B or DH5 alpha using methods that are well known. The transformation reactions are plated onto LB agar plates containing a selective antibiotic (ampicillin) and grown for 14-18 hours at 37° C. All of the transformed bacteria are pooled together, and plasmid DNA is isolated using well known methods.

Preparation of buffers mentioned in the above description of preferred methods according to the present invention will be evident to those of skill.

### Example 7

### Introduction of Vaccinia Virus DNA Fragments and Transfer Plasmids into Tissue Culture Cells for Trimolecular Recombination

A cDNA or other library is constructed in the 4 transfer plasmids as described in Example 5, or by other art-known techniques. Trimolecular recombination is employed to transfer this cDNA library into vaccinia virus. Confluent monolayers of BSC1 cells are infected with fowlpox virus HP1 at a moi of 1-1.5. Infection is done in serum free media supplemented with 0.1% Bovine Serum Albumin. The BSC1 cells may be in 12 well or 6 well plates, 60 mm or 100 mm tissue culture plates, or 25 cm², 75 cm², or 150 cm² flasks. Purified DNA from v7.5/tk or vEL/tk is digested with restriction endonucleases ApaI and NotI. Following these digestions the enzymes are heat inactivated, and the digested vaccinia arms are purified using a centricon 100 column. Transfection complexes are then formed between the digested vaccinia DNA and the transfer plasmid cDNA library. A preferred method uses Lipofectamine or Lipofectamine Plus (Life Technologies, Inc.) to form these transfection complexes. Transfections in 12 well plates usually require 0.5 micrograms of digested vaccinia DNA and 10 ng to 200 ng of plasmid DNA from the library. Transfection into cells in larger culture vessels requires a proportional increase in the amounts of vaccinia DNA and transfer plasmid. Following a two hour infection at 37° C. the fowlpox is removed, and the vaccinia DNA, transfer plasmid transfection complexes are added. The cells are incubated with the transfection complexes for 3 to 5 hours, after which the transfection complexes are removed and replaced with 1 ml DMEM supplemented with 2.5% Fetal Bovine Serum. Cells are incubated in a CO₂ incubated at 37° C. for 3 days. After 3 days the cells are harvested, and virus is released by three cycles of freeze/thaw in dry ice/isopropanol/37° C. water bath.

### Example 8

### Transfection of Mammalian Cells

This example describes alternative methods to transfect cells with vaccinia DNA and transfer plasmid. Trimolecular recombination can be performed by transfection of digested vaccinia DNA and transfer plasmid into host cells using for example, calcium-phosphate precipitation (F. L. Graham, A. J. Van der Eb (1973) Virology 52: 456-467, C. Chen, H. Okayama (1987) Mol. Cell. Biol. 7: 2745-2752), DEAE-Dextran (D. J. Sussman, G. Milman (1984) Mol. Cell. Biol. 4: 1641-1643), or electroporation (T. K. Wong, E. Neumann (1982) Biochem. Biophys. Res. Commun. 107: 584-587, E. Neumann, M. Schafer-Ridder, Y. Wang, P. H. Hofschneider (1982) EMBO J. 1: 841-845).

### Example 9

### Construction of MVA Trimolecular Recombination Vectors

In order to construct a Modified Vaccinia Ankara (MVA) vector suitable for trimolecular recombination, two unique restriction endonuclease sites must be inserted into the MVA tk gene. The complete MVA genome sequence is known (GenBank U94848). A search of this sequence revealed that restriction endonucleases AscI, RsrII, SfiI, and XmaI do not cut the MVA genome. Restriction endonucleases AscI and XmaI have been selected due to the commercial availability of the enzymes, and the size of the recognition sequences, 8 bp and 6 bp for AscI and XmaI respectively. In order to introduce these sites into the MVA tk gene a construct will be made that contains a reporter gene (E. coli gusA) flanked by XmaI and AscI sites. The Gus gene is available in pCRII.Gus (M. Merchlinsky, D. Eckert, E. Smith, M. Zauderer. 1997 Virology 238: 444-451). This reporter gene construct will be cloned into a transfer plasmid containing vaccinia tk DNA flanks and the early/late 7.5k promoter to control expression of the reporter gene. The Gus gene will be PCR amplified from this construct using Gus specific primers. Gus sense 5′ ATGTTACGTCCTGTAGAAACC 3′ (SEQ ID NO:100), and Gus Antisense 5′TCATTGTTTGCCTCCCTGCTG 3′(SEQ ID NO:101). The Gus PCR product will then be PCR amplified with Gus specific primers that have been modified to include NotI and XmaI sites on the sense primer, and AscI and ApaI sites on the antisense primer. The sequence of these primers is:
NX-Gus Sense 5′ AAAGCGGCCGCCCCGGGATGTTACGTCC 3′ (SEQ ID NO:102); and
AA-Gus antisense 5′ AAAGGGCCCGGCGCGCCTCATTGTTTGCC 3′ (SEQ ID NO:103).

This PCR product will be digested with NotI and ApaI and cloned into the NotI and ApaI sites of p7.5/tk (M. Merchlinsky, D. Eckert, E. Smith, M. Zauderer. 1997 Virology 238:444-451). The 7.5k-XmaI-gusA-AscI construct will be introduced into MVA by conventional homologous recombination in permissive QT35 or BHK cells. Recombinant plaques will be selected by staining with the Gus substrate X-Glu (5-bromo-3 indoyl-β-D-glucuronic acid; Clontech) (M. W. Carroll, B. Moss. 1995 Biotechniques 19:352-355). MVA-Gus clones, which will also contain the unique XmaI and AscI sites, will be plaque purified to homogeneity. Large scale cultures of MVA-Gus will be amplified on BHK cells, and naked DNA will be isolated from purified virus. After digestion with XmaI and AscI the MVA-Gus DNA can be used for trimolecular recombination in order to construct cDNA expression libraries in MVA.

MVA is unable to complete its life cycle in most mammalian cells. This attenuation can result in a prolonged period of high levels of expression of recombinant cDNAs, but viable MVA cannot be recovered from infected cells. The inability to recover viable MVA from selected cells would prevent the repeated cycles of selection required to isolate functional cDNA recombinants of interest. A solution to this problem is to infect MVA infected cells with a helper virus that can complement the host range defects of MVA. This helper virus can provide the gene product(s) which MVA lacks that are essential for completion of its life cycle. It is unlikely that another host range restricted helper virus, such as fowlpox, would be able to complement the MVA defect(s), as these viruses are also restricted in mammalian cells. Wild type strains of vaccinia virus would be able to complement MVA. In this case however, production of replication competent vaccinia virus would complicate additional cycles of selection and isolation of recombinant MVA clones. A conditionally defective vaccinia virus could be used which could provide the helper function needed to recover viable MVA from mammalian cells under nonpermissive conditions, without the generation of replication competent virus. The vaccinia D4R open reading frame (orf) encodes a uracil DNA glycosylase enzyme. This enzyme is essential for vaccinia virus replication, is expressed early after infection (before DNA replication), and disruption of this gene is lethal to vaccinia. It has been demonstrated that a stably transfected mammalian cell line expressing the vaccinia D4R gene was able to complement a D4R deficient vaccinia virus (G. W. Holzer, F. G. Falkner. 1997 J. Virology 71:4997-5002). A D4R deficient vaccinia virus would be an excellent candidate as a helper virus to complement MVA in mammalian cells.

In order to construct a D4R complementing cell line the D4R orf will be cloned from vaccinia strain v7.5/tk by PCR amplification using primers D4R-Sense 5′ AAAGGATCCA TAATGAATTC AGTGACTGTA TCACACG 3′ (SEQ ID NO:104), and D4R Antisense 5′ CTTGCGGCCG CTTAATAAAT AAACCCTTGA GCCC 3′(SEQ ID NO:105). The sense primer has been modified to include a BamHI site, and the anti-sense primer has been modified to include a NotI site. Following PCR amplification and digestion with BamHI and NotI the D4R orf will be cloned into the BamHI and NotI sites of pIRESHyg (Clontech). This mammalian expression vector contains the strong CMV Immediate Early promoter/Enhancer and the ECMV internal ribosome entry site (IRES). The D4RIRESHyg construct will be transfected into BSC1 cells and transfected clones will be selected with hygromycin. The IRES allows for efficient translation of a polycistronic mRNA that contains the D4Rorf at the 5′ end, and the Hygromycin phosphotransferase gene at the 3′ end. This results in a high frequency of Hygromycin resistant clones being functional (the clones express D4R). BSC1 cells that express D4R (BSC1.D4R) will be able to complement D4R deficient vaccinia, allowing for generation and propagation of this defective strain.

To construct D4R deficient vaccinia, the D4R orf (position 100732 to 101388 in vaccinia genome) and 983 bp (5′ end) and 610 bp (3′end) of flanking sequence will be PCR amplified from the vaccinia genome. Primers D4R Flank sense 5′ ATTGAGCTCT TAATACTTTT GTCGGGTAAC AGAG 3′ (SEQ ID NO:106), and D4R Flank antisense 5′ TTACTCGAGA GTGTCGCAAT TTGGATTTT 3′ (SEQ ID NO:107) contain a SacI (Sense) and XhoI (Antisense) site for cloning and will amplify position 99749 to 101998 of the vaccinia genome. This PCR product will be cloned into the SacI and XhoI sites of pBluescript II KS (Stratagene), generating pBS.D4R.Flank. The D4R gene contains a unique EcoRI site beginning at nucleotide position 3 of the 657 bp orf, and a unique PstI site beginning at nucleotide position 433 of the orf. Insertion of a Gus expression cassette into the EcoRI and PstI sites of D4R will remove most of the D4R coding sequence. A 7.5k promoter-Gus expression vector has been constructed (M. Merchlinsky, D. Eckert, E. Smith, M. Zauderer. 1997 Virology 238:444-451). The 7.5-Gus expression cassette will be isolated from this vector by PCR using primers 7.5 Gus Sense 5′ AAAGAATTCC TTTATTGTCA TCGGCCAAA 3′ (SEQ ID NO:108) and 7.5Gus antisense 5′ AATCTGCAGT CATTGTTTGC CTCCCTGCTG 3′ (SEQ ID NO:109). The 7.5Gus sense primer contains an EcoRI site and the 7.5Gus antisense primer contains a PstI site. Following PCR amplification the 7.5Gus molecule will be digested with EcoRI and PstI and inserted into the EcoRI and PstI sites in pBS.D4R.Flank, generating pBS.D4R−/7.5Gus+. D4R−/Gus+ vaccinia can be generated by conventional homologous recombination by transfecting the pBS.D4R−/7.5Gus+ construct into v7.5/tk infected BSC1.D4R cells. D4R−/Gus+ virus can be isolated by plaque purification on BSC1.D4R cells and staining with X-Glu. The D4R− virus can be used to complement and rescue the MVA genome in mammalian cells.

In a related embodiment, the MVA genome may be rescued in mammalian cells with other defective poxviruses, and also by a psoralen/UV-inactivated wild-type poxviruses. Psoralen/UV inactivation is discussed herein.

### Example 10

### Attenuation of Poxvirus Mediated Host Shut-Off by Reversible Inhibition of DNA Synthesis

As discussed infra, attenuated or defective virus is sometimes desired to reduce cytopathic effects. Cytopathic effects during viral infection might interfere with selection and identification of immunoglobulin molecules using methods which take advantage of host cell death (e.g. apoptosis induced by cross-linking). Such effects can be attenuated with a reversible inhibitor of DNA synthesis such as hydroxyurea (HU) (Pogo, B. G. and S. Dales Virology, 1971. 43(1):144-51). HU inhibits both cell and viral DNA synthesis by depriving replication complexes of deoxyribonucleotide precursors (Hendricks, S. P. and C. K. Mathews J Biol Chem, 1998. 273(45):29519-23). Inhibition of viral DNA replication blocks late viral RNA transcription while allowing transcription and translation of genes under the control of early vaccinia promoters (Nagaya, A., B. G. Pogo, and S. Dales Virology, 1970. 40(4):1039-51). Thus, treatment with reversible inhibitor of DNA synthesis such as HU allows the detection of effects of cross-linking. Following appropriate incubation, HU inhibition can be reversed by washing the host cells so that the viral replication cycle continues and infectious recombinants can be recovered (Pogo, B. G. and S. Dales Virology, 1971. 43(1):144-51).

As described below, induction of type X collagen synthesis, a marker of chondrocyte differentiation, in C3H10T ½ progenitor cells treated with BMP-2 (Bone Morphogenetic Protein-2) was blocked by vaccinia infection but that its synthesis was rescued by HU mediated inhibition of viral DNA synthesis. When HU is removed from cultures by washing with fresh medium, viral DNA synthesis and assembly of infectious particles proceeds rapidly so that infectious viral particles can be isolated as soon as 2 hrs post-wash.

C3H10T ½ cells were infected with WR vaccinia virus at MOI=1 and 1 hour later either medium or 400 ng/ml of BMP-2 in the presence or absence of 2 mM HU was added. After a further 21 hour incubation at 37LHU was removed by washing with fresh medium. The infectious cycle was allowed to continue for another 2 hours to allow for initiation of viral DNA replication and assembly of infectious particles. At 24 hours RNA was extracted from cells maintained under the 4 different culture conditions. Northern analysis was carried out using a type X collagen specific probe. The uninduced C3H10T½ cells had a mesenchymal progenitor cell phenotype and as such did not express type X collagen. Addition of BMP-2 to normal, uninfected C3H10T ½ cells induced differentiation into mature chondrocytes and expression of type X collagen, whereas addition of BMP-2 to vaccinia infected C3H10T ½ cells failed to induce synthesis of type X collagen. In the presence of 2 mM HU, BMP-2 induced type X collagen synthesis even in vaccinia virus infected C3H10T ½ cells (data not shown).

This strategy for attenuating viral cytopathic effects is applicable to other viruses, other cell types and to selection of immunoglobulin molecules that, for example, induce apoptosis upon cross-linking.

### Example 11

### Construction of Human Single-Chain-Fv (ScFv) Antibody Libraries

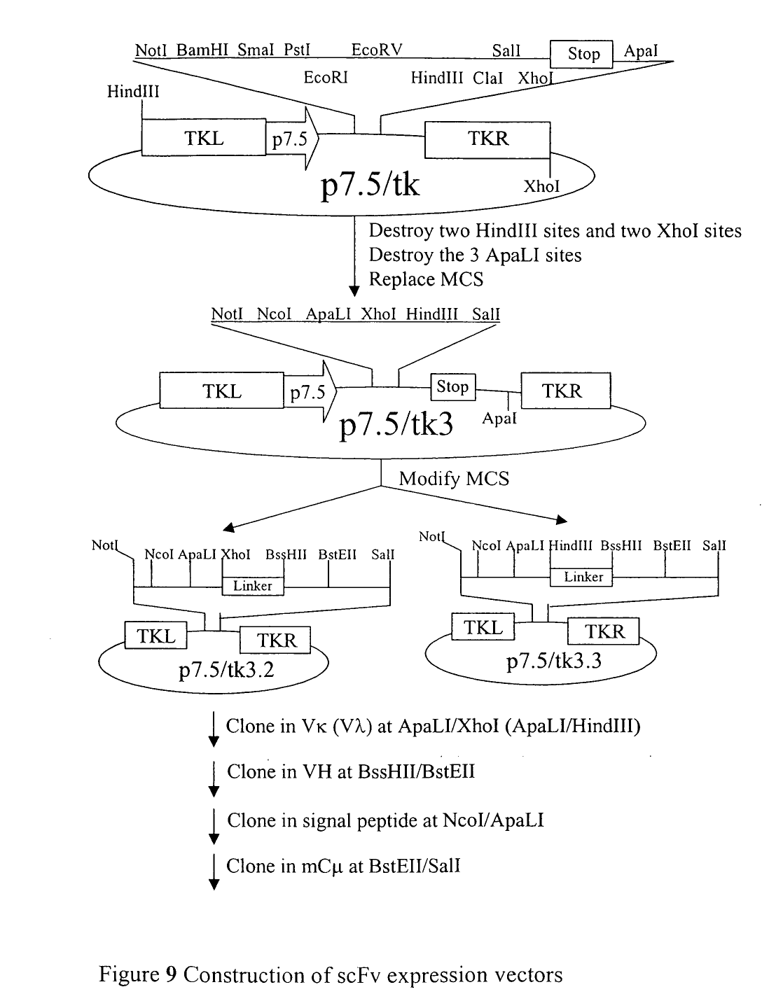

11.1 Human scFv expression vectors p7.5/tk3.2 and p7.5/tk3.3 are constructed by the following method, as illustrated in FIG. 9. Plasmid p7.5/tk3 is produced as described in Example 1.3, supra. Plasmid p7.5/tk3 is converted to p7.5/tk3.1 by changing the four nucleotides ATAC between NcoI and ApaLI sites into ATAGC, so that the ATG start codon in NcoI is in-frame with ApaLI without the inserted signal peptide. This is conveniently accomplished by replacing the NotI-to-SalI cassette described in Example 1.3 of U.S. Patent Application Publication No. U.S. Pat. No. 2,002,0018785A1, published Sep. 5, 2002 with a cassette having the sequence 5′-GCGGCCGCCC ATGGATAGCG TGCACTTGAC TCGAGAAGCT TAGTAGTCGA C-3′, referred to herein as SEQ ID NO:110.

Plasmid p7.5/tk3.1 is converted to p7.5/tk3.2 by substituting the region between XhoI and SalI (i.e., nucleotides 30 to 51 of SEQ ID NO:110), referred to herein as SEQ ID NO:111, with the following cassette: XhoI-(nucleotides encoding amino acids 106-107 of Vκ)-(nucleotides encoding a 10 amino acid linker)-G-BssHII-ATGC-BstEII-(nucleotides encoding amino acids 111-113 of VH)-stop codon-SalI. This is accomplished by digesting p7.5/tk3.1 with XhoI and SalI, and inserting a cassette having the sequence 5′CTCGAGAT CAAAGAGGGT AAATCTTCCG GATCTGGTTC CGAAGGCGCG CATGCGGTCA CCGTCTCCTC ATGAGTCGAC 3′, referred to herein as SEQ ID NO:112. The linker between Vκ and VH will have a final size of 14 amino acids, with the last 4 amino acids contributed by the VH PCR products, inserted as described below. The sequence of the linker is 5′GAG GGT AAA TCT TCC GGA TCT GGT TCC GAA GGC GCG CAC TCC 3′ (SEQ ID NO:113), which encodes amino acids EGKSSGSGSEGAHS (SEQ ID NO:114).

Plasmid p7.5/tk3.1 is converted to p7.5/tk3.3 by substituting the region between HindIII and SalI (i.e., nucleotide 36 to 51 of SEQ ID NO: 110), referred to herein as SEQ ID NO:115, with the following cassette: HindIII-(nucleotides encoding amino acid residues 105-107 of V<)-(nucleotides encoding a 10 amino acid linker)-G-BssHII-ATGC-BstEII-(nucleotides encoding amino acids 111-113 of VH)-stop codon-SalI. This is accomplished by digesting p7.5/tk3.1 with HindIII and SalI, and inserting a cassette having the sequence 5′AAGCTTACCG TCCTAGAGGG TAAATCTTCC GGATCTGGTTC CGAAGGCGCG CATGCGGTCA CCGTCTCCTC ATGAGTCGAC 3′ (SEQ ID NO:116). The linker between Vλ and VH will have a final size of 14 amino acids, with the last 4 amino acids contributed by the VH PCR products, inserted as described below. The sequence of the linker is 5′GAG GGT AAA TCT TCC GGA TCT GGT TCC GAA GGC GCG CAC TCC 3′ (SEQ ID NO:117), which encodes amino acids EGKSSGSGSEGAHS (SEQ ID NO:118).

11.2 Cytosolic Forms of scFv. Expression vectors encoding scFv polypeptides comprising human kappa or lambda immunoglobulin light chain variable regions, fused in frame with human heavy chain variable regions, are constructed as follows.

(a) Cytosolic VκVH scFv expression products are prepared as follows. Kappa light chain variable region (V-Kappa) PCR products (amino acids (−3) to (105)), produced as described in Example 1.4(b), using the primers listed in Tables 1 and 2, are cloned into p7.5/tk3.2 between the ApaLI and XhoI sites. Because of the overlap between the kappa light chain sequence and the restriction enzyme sites selected, this results in construction of a contiguous kappa light chain in the same translational reading frame as the downstream linker. Heavy chain variable region (VH) PCR products (amino acids (−4) to (110)), produced as described in Example 1.4(a), using the primers listed in Tables 1 and 2, are cloned between the BssHII and BstEII sites of p7.5/tk3.2 to form complete scFv open reading frames. The resulting products are cytosolic forms of V-Kappa-VH fusion proteins connected by a linker of 14 amino acids. The scFv is also preceded by 6 extra amino acids at the amino terminus encoded by the restriction sites and part of the V-Kappa signal peptide.

(b) Cytosolic VλVH scFv expression products are prepared as follows. Lambda light chain variable region (V-Lambda) PCR products (amino acids (−3) to (104)), produced as described in Example 1.4(c), using the primers listed in Tables 1 and 2, are cloned into p7.5/tk3.3 between the ApaLI and HindIII sites. Because of the overlap between the lambda light chain sequence and the restriction enzyme sites selected, this results in construction of a contiguous lambda light chain in the same translational reading frame as the downstream linker. Heavy chain variable region (VH) PCR products (amino acids (−4) to (110)), produced as described in Example 1.4(a), using the primers listed in Tables 1 and 2, are cloned between BssHII and BstEII sites of p7.5/tk3.3 to form complete scFv open reading frames. The resulting products are cytosolic forms of V-Lambda-VH fusion proteins connected by a linker of 14 amino acids. The scFv is also preceded by 6 extra amino acids at the amino terminus encoded by the restriction sites and part of the V-Lambda signal peptide.

11.3 Secreted or Membrane Bound Forms of scFv. The cytosolic scFv expression vectors described in section 13.2 serve as the prototype vectors into which secretion signals, transmembrane domains, cytoplasmic domains, or combinations thereof can be cloned to target scFv polypeptides to the cell surface or the extracellular space. Examples of signal peptides and membrane anchoring domains are shown in Table 7, supra. To generate scFv polypeptides to be secreted into the extracellular space, a cassette encoding an in-frame secretory signal peptide is inserted so as to be expressed in the N-terminus of scFv polypeptides between the NcoI and ApaLI sites of p7.5/tk3.2 or p7.5/tk3.3. To generate membrane-bound scFv for Ig-crosslinking or Ig-binding based selection, in addition to the signal peptide, a cassette encoding the membrane-bound form of Cμ is cloned into the C-terminus of scFv between the BstEII and SalI sites, downstream of and in-frame with the nucleotides encoding amino acids 111-113 of VH. A cytoplasmic domain may also be added.

### Example 12

### Construction of Camelized Human Single-Domain Antibody Libraries

Camelid species use only heavy chains to generate antibodies, which are termed heavy chain antibodies. The poxvirus expression system is amendable to generate both secreted and membrane-bound human single-domain libraries, wherein the human VH domain is “camelized,” i.e., is altered to resemble the V_{H}H domain of a camelid antibody, which can then be selected based on either functional assays or Ig-crosslinking/binding. Human VH genes are camelized by standard mutagenesis methods to more closely resemble camelid V_{H}H genes. For example, human VH3 genes, produced using the methods described in Example 1.4 using appropriate primer pairs selected from Tables 1 and 2, is camelized by substituting G44 with E, L45 with R, and W47 with G or I. See, e.g., Riechmann, L., and Muyldermans, S. J. Immunol. Meth. 231:25-38. To generate a secreted single-domain antibody library, cassettes encoding camelized human VH genes are cloned into pVHEs (produced as described in example 1 of U.S. Patent Application Publication No. U.S. Pat. No. 2,002,0018785A1, published Sep. 5, 2002 and PCT Publication WO 02102855, published Dec. 27, 2002, each incorporated herein by reference), to be expressed in-frame between the BssHII and BstEII sites. To generate a membrane-bound single-domain antibody library, cassettes encoding camelized human VH genes are cloned into pVHE, produced as described in Example 1.1, to be expressed in-frame between the BssHII and BstEII sites. Vectors pVHE and pVHEs already have the signal peptide cloned in between the NcoI and BssHII sites. Amino acid residues in the three CDR regions of the camelized human VH genes are subjected to extensive randomization, and the resulting libraries can be selected in poxviruses as described herein.

### Example 13

### Construction of a Diverse Library of High Affinity Human Antibodies

The current invention is the only available method for the identification and production of a diverse library of antibodies, e.g., bispecific antibodies of the present invention, in vaccinia or other pox viruses. The vaccinia vector can be designed to give high levels of membrane receptor expression to allow efficient binding to an antigen coated matrix. Alternatively, the recombinant immunoglobulin heavy chain genes can be engineered to induce apoptosis upon crosslinking of receptors by antigen. Since vaccinia virus can be readily and efficiently recovered even from cells undergoing programmed cell death, the unique properties of this system make it possible to rapidly select specific human antibody genes.

Optimal immunoglobulin heavy and light chains are selected sequentially, which maximizes diversity by screening all available heavy and light chain combinations. The sequential screening strategy is to at first select an optimal heavy chain from a small library of 10⁵H-chain recombinants in the presence of a small library of 10⁴ diverse light chains. This optimized H-chain is then used to select an optimized partner from a larger library of 10⁶ to 10⁷ recombinant L-chains. Once an optimal L-chain is selected, it is possible to go back and select a further optimized H-chain from a larger library of 10⁶ to 10⁷ recombinant H-chains. This reiteration is a boot-strap strategy that allows selection of a specific high-affinity antibody from as many as 10¹⁴H₂L₂ of H₄L₄ combinations. In contrast, selection of single chain Fv in a phage library or of Fab comprised of separate VH-CH1 and VL-CL genes encoded on a single plasmid is a one step process limited by the practical size limit of a single phage library—perhaps 10¹¹ phage particles.

Since it is not feasible to screen 10¹⁴ combinations of 10⁷ H chains and 10⁷ L chains, the selection of optimal H chains begins from a library of 10⁵ H chain vaccinia recombinants in the presence of 10⁴ L chains in a non-infectious vector. These combinations will mostly give rise to low affinity antibodies against a variety of epitopes and result in selection of e.g., 1 to 100 different H chains. If 100 H chains are selected for a basic antibody, these can then be employed in a second cycle of selection with a larger library of 10⁶ or 10⁷ vaccinia recombinant L chains to pick 100 optimal L chain partners. The original H chains are then set aside and the 100 L chains are employed to select new, higher affinity H chains from a larger library of 10⁶ or 10⁷ H chains.

The strategy is a kind of in vitro affinity maturation. As is the case in normal immune responses, low affinity antibodies are initially selected and serve as the basis for selection of higher affinity progeny during repeated cycles of immunization. Whereas higher affinity clones may be derived through somatic mutation in vivo, this in vitro strategy achieves the same end by the re-association of immunoglobulin chains. In both cases, the partner of the improved immunoglobulin chain is the same as the partner in the original lower affinity antibody.

The basis of the strategy is leveraging the initial selection for a low affinity antibody. It is essential that a low affinity antibody be selected. The vaccinia-based method for sequential selection of H and L chains is well-suited to insure that an initial low affinity selection is successful because it has the avidity advantage that comes from expressing bivalent antibodies. In addition, the level of antibody expression can be regulated by employing different promoters in the vaccinia system. For example, the T7 polymerase system adapted to vaccinia gives high levels of expression relative to native vaccinia promoters. Initial rounds of selection can be based on a high level T7 expression system to insure selection of a low affinity “basic antibody” and later rounds of selection can be based on low level expression to drive selection of a higher affinity derivative.

Multispecific, e.g., bispecific antibodies are identified by methods described herein. The final antibody product is optimized by selection of a fully assembled multispecific antibody rather than a single chain Fv. That is, selection is based on bivalent (H₂L₂) or tetravalent (H₄L₄) antibodies rather than scFv or Fab fragments. Synthesis and assembly of fully human, complete antibodies occurs in mammalian cells allowing immunoglobulin chains to undergo normal post-translational modification and assembly.

A relatively wide range of antibody epitope specificities can be identified, including specificities on the basis of activity in a target cell. Specifically, antibodies can be selected on the basis of specific physiological effects on target cells (e.g., screening for inhibition of TNF-secretion by activated monocytes; induction of apoptosis; etc.) An outline of the method for screening for specific on the basis of a monospecific bivalent in cell-based assay is as follows:
1. An immunoglobulin heavy chain cDNA library in secretory form is constructed from naïve human lymphocytes in a vaccinia virus vector prepared according to the methods described herein. Multiple pools of, for example, about 100 to about 1000 recombinant viruses, are separately expanded and employed to infect producer cells at dilutions such that on average each cell is infected by one immunoglobulin heavy chain recombinant virus. These same cells are also infected with psoralin inactivated immunoglobulin light chain recombinant vaccinia virus from an immunoglobulin light chain library constructed in the same vaccinia virus vector. Alternatively, the infected cells may be transfected with immunoglobulin light chain recombinants in a plasmid expression vector. In the population of cells as a whole, each heavy chain can be associated with any light chain.
2. Infected cells are incubated for a time sufficient to allow secretion of fully assembled antibodies.
3. Assay wells are set up in which indicator cells of functional interest are incubated in the presence of aliquots of secreted antibody. These might, for example, include activated monocytes secreting TNFα. A simple ELISA assay for TNFα may then be employed to screen for any pool of antibodies that includes an activity that inhibits cytokine secretion.
4. Individual members of the selected pools are further analyzed to identify the relevant immunoglobulin heavy chain.
5. Once specific antibody heavy chains have been selected, the entire procedure is repeated with an immunoglobulin light chain cDNA library constructed in the proprietary vaccinia vector in order to select specific immunoglobulin light chains that contribute to optimal antigen binding.
6. The MAb sequences are identified and specific binding verified through standard experimental techniques. Because functional selection does not require a priori knowledge of the target membrane receptor, the selected Mab is both a potential therapeutic and a discovery tool to identify the relevant membrane receptor.

Based on disclosures elsewhere herein, one of ordinary skill in the art could readily apply these methods of bispecific antibodies of the present invention, either bivalent or tetravalent. Selection occurs within human cell cultures following random association of immunoglobulin heavy and light chains. As noted above, this avoids repertoire restrictions due to limitations of synthesis in bacteria. It also avoids restrictions of the antibody repertoire due to tolerance to homologous gene products in mice. Mouse homologs of important human proteins are often 80% to 85% identical to the human sequence. It should be expected, therefore, that the mouse antibody response to a human protein would primarily focus on the 15% to 20% of epitopes that are different in man and mouse. This invention allows efficient selection of high affinity, fully human antibodies with a broad range of epitope specificities. The technology is applicable to a wide variety of projects and targets including functional selection of antibodies to previously unidentified membrane receptors with defined physiological significance.

### Example 14

### A. Strategy for Generating Highly Diverse Variable Region Libraries

Libraries have been produced from bone marrow to take advantage of the presence of immature B cells and pre B cells prior to negative selection. These libraries are of sufficient complexity, with respect to variable region diversity, that it will be relatively easy to isolate low affinity antibodies to any antigen.

During an antigen-driven response in the intact animal, antigen-selected B cells diversify their V genes through the process of somatic hyper mutation (SHM). SHM occurs in a specialized lymphoid structure called a germinal center (gc), found in all lymphoid organs, which is formed by one to three antigen-specific B cells. The human palatine tonsil is one source of gcs. See, e.g., Nave, H. et al., Anat. Embrol. 204: 367-373 (2001) and Klein, U. et al., Proc. Nat. Acad. Sci. 100(5): 2639-2644 (2003), which are hereby incorporated by reference in their entireties.

Mutating gc B cells (called “centroblasts”) proliferate at a very high rate while mutations accumulate in their V genes. In centroblasts, mutation within V genes is random with respect to the original antigen. Centroblasts develop into non-cycling centrocytes, which, based on their ability to express high-affinity antibody mutants, differentiate into memory B cells or plasma cells. Klein, U. et al., Proc. Nat. Acad. Sci. 100(5): 2639-2644 (2003). Centrocytes re-express membrane antibody receptors and are capable of interacting with antigen presenting cells in the gc. Centrocytes can re-enter the cell cycle as centroblasts, and are capable of undergoing further somatic mutation.

About 90% or more of the mutations in the V-genes of gc B cells are deleterious and result in loss of specific binding to the antigen which generated the SHM response. Those gc B cells that lose antigen specificity normally undergo apoptosis; however, the RNA and/or DNA is isolated from those cells by methods which are well known to those of skill in the art and described herein, before the cells die. Therefore, in addition to a library of variable regions specific to the antigen which initiated the SHM response, a diverse library of variable regions that are specific to potentially numerous other antigens is generated.

Highly diverse VH and VL libraries are produced from gc centroblasts and/or centrocytes according to the methods described in Example 1, above. CD38 positive and CD19 positive centroblasts and/or centrocytes are isolated from lymphoid tissue by flow cytometry (Pascual V., Liu, Y. J., Magalski, A., de Bouteiller, O., Banchereau, J. and Capra, J. D. J. Exp. Med. 180: 329-339 (1994)). Centroblast and/or centrocyte genomic DNA libraries are produced as described in Example 1, supra, except that the libraries are generated using the PCR amplified products from centroblasts and/or centrocytes. Primer pairs for use in the PCR amplifications of the variable regions are the same as those used to amplify the variable regions from any B cell, as described herein. The primers used to amplify the variable regions are listed in Tables 1 and 2.

VH genes carried by centroblasts and/or centrocytes represent a randomly diversified set of CDR1 and CDR2. However, the diversity of DH and JH utilization and CDR3 length in VH regions is restricted, as germinal centers are generated by very few B cells. VH genes from naïve B cells have limited CDR1 and CDR2 diversity, but significant CDR3 length and functional diversity by virtue of having been derived from many VDJ rearrangements. Thus, in another embodiment, a recombinant VH library of even higher diversity is generated by incorporating VH (CDR1 and CDR2) from centroblasts and/or centrocytes and DH-JH (CDR3) from naïve B cells. Naïve B-cell cDNA is produced based on known methods of nucleic acid isolation, purification and reverse transcription from poly-A selected RNA.

Each of the major VH gene families (VH1, VH3 and VH4) have a highly conserved region of approximately 21 nucleotides in FR3. This region (designated FR3C) accumulates few mutations during an immune response. Centroblast and/or centrocyte VH nucleic acid segments encoding at least CDR1 and CDR2 are amplified from centroblast and/or centrocyte cDNA using a VH family upstream primer, for example, VHla, VH2a, VH3a, VH4a, or VH5a (Table 2), and an FR3C downstream primer, for example, VH1 FR3C downstream, VH3 FR3Ca downstream, VH3 FR3Cb downstream, VH3 FR3 Cc downstream, or VH4 FR3C downstream (Table 8). Nucleic acid segments encoding DJ (CDR3) regions are amplified from naïve B-cell cDNA using an FR3C upstream primer, for example, VH1 FR3C upstream, VH3 FR3Ca upstream, VH3 FR3Cb upstream, VH3 FR3 Cc upstream, or VH4 FR3C upstream (Table 8), and a JH consensus downstream primer, for example, JH1a, JH2a, JH3a, JH4/5a, or JH6a (Table 2).

These PCR products are then combined, denatured, reannealed and filled in using DNA polymerase. The resulting products are comprised of three nucleic acid segments: the original naïve B-cell PCR amplified products, the centroblast and/or centrocyte PCR amplified products, and a third product representing a full length hybrid VH nucleic acid segment encoding a centroblast VH and naïve B-cell DJ. This third species is separated by size from the other two products and used to generate a VH library by methods described in Example 1.

TABLE 8

Oligonucleotide Primers for PCR amplification
of human immunoglobulin variable regions.
Primer sequences are from 5′ to 3′.

Primers

| | | |
|---|---|---|
| VH1 FR3C upstream | (SEQ ID NO:119) | CACAGCCTACATGGAGCTGAGCAG |
| VH1 FR3C downstream | (SEQ ID NO:120) | CTGCTCAGCTCCATGTAGGCTGTG |
| VH3 FR3Ca upstream | (SEQ ID NO:121) | CTGTATCTGCAAATGAACAGCCTG |
| VH3 FR3Ca downstream | (SEQ ID NO:122) | CAGGCTGTTCATTTGCAGATACAG |
| VH3 FR3Cb upstream | (SEQ ID NO:123) | CTGTATCTGCAAATGAACAGTCTG |
| VH3 FR3Cb downstream | (SEQ ID NO:124) | CAGACTGTTCATTTGCAGATACAG |
| VH3 FR3Cc upstream | (SEQ ID NO:125) | CTGTATCTTCAAATGAACAGCCTG |
| VH3 FR3Cc downstream | (SEQ ID NO:126) | CAGGCTGTTCATTTGAAGATACAG |
| VH4 FR3C upstream | (SEQ ID NO:127) | CAGTTCTCCCTGAAGCTGAGCTCTGTG |
| VH4 FR3C downstream | (SEQ ID NO:126) | CACAGAGCTCAGCTTCAGGGAGAACTG |

### B. Comparison of Diversity Between Germinal Center B Cell-Derived Library and Normal Bone Marrow B Cell-Derived Library

Two libraries of human immunoglobulin heavy chains paired with a single pre-selected light chain (3E10 VK) for antibodies to a breast cancer antigen (C35) were screened. See Example 3.6, supra. C35 is a human gene that is differentially expressed in human carcinoma. See U.S. Patent Application Publication No. 2002/0155447 A1, published Oct. 24, 2002 (U.S. Ser. No. 09/824,787, filed Apr. 4, 2001), which is hereby incorporated by reference in its entirety. The VH of the first library were derived from gc centroblasts and centrocytes isolated by flow cytometry as described, supra, in section A of this Example. The VH of the second library were derived from normal bone marrow B cells. VH regions were prepared from normal bone marrow B cells as described in Example 1.4, above. VH regions were prepared from gc B cells (i.e., centroblasts and centrocytes) isolated from tonsils, see Klein et al., Proc. Nat. Acad. Sci. 100 (5): 2639-2644 (2003).

To amplify V genes from genomic DNA obtained from tonsil germinal center centroblasts and centrocytes, the following PCR set ups were employed.

For amplification of VH genes, each pcr reaction contained, in a total volume of 30 μl, 19.52 μl dH2O, 3 μl 10×PCR reaction buffer, 1.8 μl 10 mM dNTP, 2.4 μl of 50 mM VH primer, 0.6 μl of 50 μM JH primer pool, 2.23 μl germinal center DNA (50,000 cell equivalents), and 0.45 μl thermostable DNA polymerase. The JH primer pool contained 2% JH1, 2% JH2, 8% JH3, 68% JH4/5, and 20% JH6, reflecting the relative utilization of each JH gene segment in the antibody repertoire. Brezinschek, H. P. et al., J. Immunol. 155, 190-202 (1995); Foster, S. J. et al., J. Clin. Invest. 99, 1614-1627 (1997)).

TABLE 9
PCR set up for amplification of VH genes

| Step | Temp | Time | |
|---|---|---|---|
| 1 | 95° C. | 4 | min. |
| 2 | 95° C. | 45 | sec. |
| 3 | 55° C. | 45 | sec. |
| 4 | 72° C. | 1 | min. |
| 5 | 95° C. | 45 | sec. |
| 6 | 72° C. | 1 | min. |
| | | 45 | sec. |
| 7 | Repeat steps 5 through 6 for 27 cycles | | |
| 8 | 72° C. | 4 | min. |

| | | |
|---|---|---|
| 9 | 4° C. | indefinitely |

For amplification of VK genes, each PCR reaction contained in a total volume of 30 μl, 19.52 μl dH2O, 3 μl 10×PCR reaction buffer, 1.8 μl 10 mM dNTP, 2.4 μl of 50 mM VK primer, 0.6 μl of 50 μM JK primer pool, 2.23 μl germinal center DNA (50,000 cell equivalents), and 0.45 μl thermostable DNA polymerase.

TABLE 10
PCR set up for amplification of VK genes

| Step | Temp | Time | |
|---|---|---|---|
| 1 | 95° C. | 4 | min. |
| 2 | 95° C. | 45 | sec. |
| 3 | 55° C. | 45 | sec. |
| 4 | 72° C. | 1 | min. |
| 5 | Repeat steps 2 through 4 for 1 cycle | | |
| 6 | 95° C. | 45 | sec. |
| 7 | 72° C. | 1 | min. |
| | | 45 | sec. |
| 8 | Repeat steps 6 through 7 for 27 cycles | | |
| 9 | 72° C. | 4 | min. |

| | | |
|---|---|---|
| 10 | 4° C. | indefinitely |

Vaccinia virus libraries which express secreted heavy chain subunit polypeptides encoded by polynucleotides comprising VH genes from bone marrow cells or gc were constructed by the methods of Example 1. The library was used to infect 384 pools of cells at a MOI of about 1 in 96-well tissue culture plates. These cells were then coinfected with psoralin-treated vaccinia viruses expressing 3E10VK.

The antibodies were expressed as secreted IgG and assayed by ELISA for specificity, as described supra. There were, on average, 100,000 cells and 100 different heavy chains in each pool. That is, about 9,600 different antibodies were screened in each plate, with a total of 384 pools in 4 plates for germinal center cells and 440 pools in 5 plates for bone marrow B cells. Each pool, or “mini library,” from which the conditioned medium registered positive by ELISA was assumed to contain vaccinia virus vectors expressing one unique C35-specific antibody. Even with this relatively small number of antibody clones, the results showed that, whereas only one C35-specific antibody was detected from 440 different bone marrow mini libraries, there were at least 11 different C35-specific antibodies detected from 384 germinal center mini libraries (data not shown). These results verify that the isolation of nucleic acid segments encoding heavy chain variable regions from centrocytes and centroblasts affords a surprising increase in library diversity over nucleic acid segments encoding heavy chain variable regions isolated from bone marrow.

The present invention is not to be limited in scope by the specific embodiments described which are intended as single illustrations of individual aspects of the invention, and any constructs, viruses or enzymes which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference. The disclosure and claims of U.S. application Ser. No. 08/935,377, filed Sep. 22, 1997 and U.S. Application No. 60/192,586, filed Mar. 28, 2000 are herein incorporated by reference.

1. A method of identifying polynucleotides which encode a bispecific antibody, or a bispecific antigen-binding fragment thereof, comprising:
(A) introducing into a population of eukaryotic host cells capable of expressing said bispecific antibody, or a bispecific antigen-binding fragment thereof, a first library of polynucleotides encoding, through operable association with a transcriptional control region, a plurality of immunoglobulin subunit polypeptides, or fragment thereof, selected from the group consisting of:
(i) first heavy chain subunit polypeptides; and
(ii) light chain subunit polypeptides;
(B) introducing into said host cells a polynucleotide encoding, through operable association with a transcriptional control region, a second heavy chain subunit polypeptide, or fragment thereof; and
(C) introducing into said host cells a polynucleotide encoding, through operable association with a transcriptional control region, an immunoglobulin subunit polypeptide, or fragment thereof, wherein said immunoglobulin subunit polypeptide is a light chain if the immunoglobulin subunit polypeptides of (A) are first heavy chains, and said immunoglobulin subunit polypeptide is a first heavy chain if the immunoglobulin subunit polypeptides of (A) are light chains;
wherein light chain subunit polypeptides combine with said first and second heavy chain subunit polypeptides to form a first heavy and light chain pair comprising a first antigen binding domain and a second heavy and light chain pair comprising a second antigen binding domain, where said first antigen binding domain and said second antigen binding domain are non-identical; and
wherein said first heavy and light chain pair is capable of combining with said second heavy and light chain pair to form a bispecific antibody, or bispecific antigen-binding fragment thereof;
(D) permitting expression of bispecific antibodies, or bispecific antigen-binding fragments thereof from said host cells;
(E) contacting said bispecific antibodies, or bispecific antigen-binding fragments thereof with an antigen comprising two non-identical epitopes; and
(F) recovering polynucleotides from said first library which encode immunoglobulin subunit polypeptides which, as part of a bispecific antibody, or bispecific antigen-binding fragment thereof, bind to said antigen, wherein said first and second antigen binding domains each bind to one of said two non-identical epitopes.

2. A method of identifying polynucleotides which encode a bispecific, bivalent antibody, or a bispecific antigen-binding fragment thereof, comprising:
(A) introducing into a population of eukaryotic host cells capable of expressing said bispecific, bivalent antibody, or a bispecific antigen-binding fragment thereof, a first library of polynucleotides encoding, through operable association with a transcriptional control region, a plurality of immunoglobulin subunit polypeptides, or fragment thereof, selected from the group consisting of:
(i) first heavy chain subunit polypeptides, each polypeptide of a type comprising
(a) a first heavy chain constant region, said constant region comprising a first heterodimerization domain,
(b) a first heavy chain variable region fused to the N-terminus of said first heavy chain constant region, and
(c) a signal peptide capable of directing secretion or cell surface expression of said heavy chain subunit polypeptide, fused to the N-terminus of said first heavy chain variable region; and
(ii) light chain subunit polypeptides, each polypeptide of a type comprising
(a) a light chain constant region,
(b) a light chain variable region fused to the N-terminus of said light chain constant region, and
(c) a signal peptide capable of directing secretion of said light chain subunit polypeptide, fused to the N-terminus of said light chain variable region;
(B) introducing into said host cells a polynucleotide encoding, through operable association with a transcriptional control region, a second heavy chain subunit polypeptide, or fragment thereof, of a type comprising
(a) a second heavy chain constant region, said constant region comprising a second heterodimerization domain, wherein said second heterodimerization domain interacts with said first heterodimerization domain to promote formation of a heavy chain heterodimer,
(b) a second heavy chain variable region fused to the N-terminus of said second heavy chain constant region, and
(c) a signal peptide capable of directing secretion or cell surface expression of said heavy chain subunit polypeptide, fused to the N-terminus of said second heavy chain variable region; and
(C) introducing into said host cells a polynucleotide encoding, through operable association with a transcriptional control region, an immunoglobulin subunit polypeptide, or fragment thereof, wherein said immunoglobulin subunit polypeptide is a light chain if the immunoglobulin subunit polypeptides of (A) are first heavy chains, and said immunoglobulin subunit polypeptide is a first heavy chain if the immunoglobulin subunit polypeptides of (A) are light chains;
wherein light chain subunit polypeptides combine with said first and second heavy chain subunit polypeptides to form a first heavy and light chain pair comprising a first antigen binding domain and a second heavy and light chain pair comprising a second antigen binding domain, where said first antigen binding domain and said second antigen binding domain are non-identical; and
wherein said first heavy and light chain pair combines with said second heavy and light chain pair to form a bispecific, bivalent antibody, or bispecific antigen-binding fragment thereof;
(D) permitting expression of bispecific, bivalent antibodies, or bispecific antigen-binding fragments thereof from said host cells;
(E) contacting said bispecific, bivalent antibodies, or bispecific antigen-binding fragments thereof with an antigen comprising two non-identical epitopes; and
(F) recovering polynucleotides from said first library which encode immunoglobulin subunit polypeptides which, as part of a bispecific, bivalent antibody, or bispecific antigen-binding fragment thereof, bind to said antigen, wherein said first and second antigen binding domains each bind to one of said two non-identical epitopes.

3. The method of claim 2, wherein the polynucleotide of (B) encodes a fixed second heavy chain subunit polypeptide which, as part of a defined antigen binding domain, binds to a known epitope, said epitope being identical to one of said two epitopes recited in (E).

4. The method of claim 2, wherein the polynucleotide of (B) is a member of a second library of polynucleotides encoding, through operable association with a transcriptional control region, a plurality of second heavy chain subunit polypeptides which combine with the immunoglobulin subunit polypeptides encoded by the polynucleotides of (A) and (C) to form bispecific, bivalent antibodies, or bispecific antigen-binding fragments thereof.

5. The method of claim 4, wherein the recovery of (F) further comprises recovering polynucleotides from said second library which encode a second heavy chain subunit polypeptide, wherein said second heavy chain subunit polypeptide, when combined with a light chain subunit polypeptide encoded by a polynucleotide of (A) or (C) forms an antigen binding domain specific for at least one of the two epitopes recited in (E).

6. The method of claim 2, wherein the polynucleotide of (C) encodes a fixed immunoglobulin subunit polypeptide which, as part of an antigen binding domain, binds to one of said two epitopes recited in (E).

7. The method of claim 6, wherein said fixed immunoglobulin subunit polypeptide is a first heavy chain subunit polypeptide.

8. The method of claim 6, wherein said fixed immunoglobulin subunit polypeptide is a light chain subunit polypeptide.

9. The method of claim 8, wherein said fixed light chain subunit polypeptide combines with the heavy chain subunit polypeptides encoded by the polynucleotides of (A) and (B) to form bispecific, bivalent antibodies, or bispecific antigen-binding fragments thereof.

10. The method of claim 8, wherein the polynucleotide of (B) encodes a fixed second heavy chain subunit polypeptide, and wherein said fixed light chain subunit polypeptide combines with said fixed second heavy chain subunit polypeptide to form a defined antigen binding domain which binds to a known epitope, said epitope being identical to one of said two epitopes recited in (E).

11. The method of claim 8, wherein said light chain combines with both said first heavy chain subunit polypeptide and said second heavy chain subunit polypeptide to form two non-identical antigen binding domains, each of which binds to one of said two epitopes recited in (E).

12. The method of claim 2, wherein the polynucleotide of (C) is a member of a third library of polynucleotides encoding, through operable association with a transcriptional control region, a plurality of immunoglobulin subunit polypeptides which combine with the immunoglobulin subunit polypeptides encoded by the polynucleotides of (A) and optionally, (B), to form bispecific, bivalent antibodies, or bispecific antigen-binding fragments thereof.

13. The method of claim 12, wherein the recovery of (F) further comprises recovering polynucleotides from said third library which encode an immunoglobulin subunit polypeptide, wherein said immunoglobulin subunit polypeptide, when combined with an immunoglobulin subunit polypeptide encoded by a polynucleotide of (A), and optionally, (B), forms an antigen binding domain specific for at least one of the two epitopes recited in (E).

14. The method of claim 2, further comprising:
(G) introducing the polynucleotides recovered in (F) into a population of host cells capable of expressing said bispecific, bivalent antibody, or bispecific antigen-binding fragment thereof;
(H) introducing into said host cells those polynucleotides of (B) or (C) which encode one or more immunoglobulin subunit polypeptide types not encoded by the polynucleotides of (G);
(I) permitting expression of bispecific, bivalent antibodies, or bispecific antigen-binding fragments thereof, from said host cells;
(J) contacting said bispecific, bivalent antibodies, or bispecific antigen-binding fragments thereof with the antigen of (E); and
(K) recovering polynucleotides of (G) which encode an immunoglobulin subunit polypeptide which, as part of a bispecific, bivalent antibody, or bispecific antigen-binding fragment thereof, binds to said antigen, wherein said first and second antigen binding domains each bind to one of said two epitopes recited in (E).

15. The method of claim 14, further comprising repeating steps (G)-(K) one or more times, thereby enriching for those polynucleotides of (G) which encode an immunoglobulin subunit polypeptide which, as part of a bispecific, bivalent antibody, or bispecific antigen-binding fragment thereof, bind to said antigen, wherein said first and second antigen binding domains each bind to one of said two epitopes recited in (E).

16. The method of claim 2, further comprising
(L) isolating the polynucleotides recovered in (F) or (K).

17. The method of claim 16, further comprising:
(M) introducing into a population of host cells capable of expressing said bispecific, bivalent antibody, or bispecific antigen-binding fragment thereof those polynucleotides of (B) or (C) which encode one or more immunoglobulin subunit polypeptide types not encoded by said isolated polynucleotides of (L);
(N) introducing into said host cells said isolated polynucleotides of (L);
(O) permitting expression of bispecific, bivalent antibodies, or bispecific antigen-binding fragments thereof, from said host cells;
(P) contacting said bispecific, bivalent antibodies, or bispecific antigen-binding fragments thereof with the antigen of (E); and
(Q) recovering polynucleotides of (M) which encode an immunoglobulin subunit polypeptide not encoded by said isolated polynucleotides of (L) which, as part of a bispecific, bivalent antibody, or bispecific antigen-binding fragment thereof, binds to said antigen, wherein said first and second antigen binding domains each bind to one of said two epitopes recited in (E).

18. The method of claim 17, further comprising:
(R) introducing the polynucleotides recovered in (O) into a population of host cells capable of expressing said bispecific, bivalent antibody, or bispecific antigen-binding fragment thereof;
(S) introducing into said host cells the isolated polynucleotides of (L) and those polynucleotides of (B) or (C) which encode one or more immunoglobulin subunit polypeptide types not encoded by said recovered polynucleotides of (O) or said isolated polynucleotides of (L);
(T) permitting expression of bispecific, bivalent antibodies, or bispecific antigen-binding fragments thereof, from said host cells;
(U) contacting said bispecific, bivalent antibodies, or bispecific antigen-binding fragments thereof with the antigen of (E); and
(V) recovering those polynucleotides of (R) which encode one or more immunoglobulin subunit polypeptides which, as part of a bispecific, bivalent antibody, or bispecific antigen-binding fragment thereof, bind to said antigen, wherein said first and second antigen binding domains each bind to one of said two epitopes recited in (E).

19. The method of claim 18, further comprising repeating steps (R)-(V) one or more times, thereby enriching for those polynucleotides of (R) which encode an immunoglobulin subunit polypeptides which, as part of a bispecific, bivalent antibody, or bispecific antigen-binding fragment thereof, binds to said antigen, wherein said first and second antigen binding domains each bind to one of said two epitopes recited in (E).

20. The method of claim 17, further comprising
(W) isolating the polynucleotides recovered in (O) or (V).

21. The method of claim 20, further comprising:
(X) introducing into a population of host cells capable of expressing said bispecific, bivalent antibody, or bispecific antigen-binding fragment thereof those polynucleotides of (B) or (C) which encode immunoglobulin subunit polypeptide types not encoded by said isolated polynucleotides of (L) and (W);
(Y) introducing into said host cells said isolated polynucleotides of (L) and (W);
(Z) permitting expression of bispecific, bivalent antibodies, or bispecific antigen-binding fragments thereof, from said host cells;
(AA) contacting said bispecific, bivalent antibodies, or bispecific antigen-binding fragments thereof with the antigen of (E); and
(BB) recovering polynucleotides of (X) which encode an immunoglobulin subunit polypeptide not encoded by said isolated polynucleotides of (L) and (W) which, as part of a bispecific, bivalent antibody, or bispecific antigen-binding fragment thereof, binds to said antigen, wherein said first and second antigen binding domains each bind to one of said two epitopes recited in (E).

22. The method of claim 21, further comprising:
(CC) introducing the polynucleotides recovered in (BB) into a population of host cells capable of expressing said bispecific, bivalent antibody, or bispecific antigen-binding fragment thereof;
(DD) introducing into said host cells the isolated polynucleotides of (L) and (W);
(EE) permitting expression of bispecific, bivalent antibodies, or bispecific antigen-binding fragments thereof, from said host cells;
(FF) contacting said bispecific, bivalent antibodies, or bispecific antigen-binding fragments thereof with the antigen of (E); and
(GG) recovering those polynucleotides of (CC) which encode an immunoglobulin subunit polypeptide which, as part of a bispecific, bivalent antibody, or bispecific antigen-binding fragment thereof, binds to said antigen, wherein said first and second antigen binding domains each bind to one of said two epitopes recited in (E).

23. The method of claim 22, further comprising repeating steps (CC)-(GG) one or more times, thereby enriching for those polynucleotides of (CC) which encode an immunoglobulin subunit polypeptide which, as part of a bispecific, bivalent antibody, or bispecific antigen-binding fragment thereof, binds to said antigen, wherein said first and second antigen binding domains each bind to one of said two epitopes recited in (E).

24. The method of claim 21, further comprising
(HH) isolating the polynucleotides recovered in (BB) or (GG).

25. The method of claim 2, further comprising introducing into said host cells an additional polynucleotide encoding, through operable association with a transcriptional control region, a fixed light chain subunit polypeptide which, when combined with a fixed heavy chain subunit polypeptide, forms a defined antigen binding domain which binds to a known epitope, said epitope being identical to one of said two epitopes recited in (E).

26. A method of claim 1, for identifying polynucleotides which encode a bispecific, tetravalent antibody, or a bispecific antigen-binding fragment thereof, comprising:
(At) introducing into a population of eukaryotic host cells capable of expressing said bispecific, tetravalent antibody or bispecific antigen-binding fragment thereof a first library of polynucleotides encoding, through operable association with a transcriptional control region, a plurality of immunoglobulin subunit polypeptides, or fragments thereof, selected from the group consisting of:
(i) first heavy chain subunit polypeptides, each polypeptide comprising
(a) a first heavy chain constant region, said constant region comprising a means for tetramerization,
(b) a first heavy chain variable region fused to the N-terminus of said first heavy chain constant region, and
(c) a signal peptide capable of directing secretion or cell surface expression of said first heavy chain subunit polypeptide, fused to the N-terminus of said first heavy chain variable region; and
(ii) light chain subunit polypeptides, each polypeptide comprising
(a) a light chain constant region,
(b) a light chain variable region fused to the N-terminus of said light chain constant region, and
(c) a signal peptide capable of directing secretion of said light chain subunit polypeptide, fused to the N-terminus of said light chain variable region;
(Bt) introducing into said host cells a polynucleotide encoding, through operable association with a transcriptional control region, a second heavy chain subunit polypeptide, or fragment thereof, comprising
(a) a second heavy chain constant region, said constant region comprising a means for tetramerization,
(b) a second heavy chain variable region fused to the N-terminus of said second heavy chain constant region, and
(c) a signal peptide capable of directing secretion or cell surface expression of said second heavy chain subunit polypeptide, fused to the N-terminus of said second heavy chain variable region;
(Ct) introducing into said host cells a polynucleotide encoding, through operable association with a transcriptional control region, an immunoglobulin subunit polypeptide, or fragment thereof, wherein said immunoglobulin subunit polypeptide is a light chain if the immunoglobulin subunit polypeptides of (At) is a first heavy chain, and said immunoglobulin subunit polypeptide is a first heavy chain if the immunoglobulin subunit polypeptides of (At) are light chains,
wherein two heavy chain subunit polypeptides interact via disulfide linkages to form a first heavy chain pair and two heavy chain subunit polypeptides interact via disulfide linkages to form a second heavy chain pair, wherein said first heavy chain pair comprises a first and a second heavy chain subunit polypeptide or two first heavy chain subunit polypeptides, and wherein said second heavy chain pair comprises a first and a second heavy chain subunit polypeptide or two second heavy chain subunit polypeptides;
wherein light chain subunit polypeptides combine with said first heavy chain pair to form a first bivalent antibody, or antigen-binding fragment thereof, comprising two first antigen-binding domains;
wherein light chain subunit polypeptides combine with said second heavy chain pair to form a second bivalent antibody, or antigen-binding fragment thereof, comprising two second-antigen binding domains;
wherein at least one first antigen binding domain is non-identical to at least one second antigen binding domain; and
wherein said first bivalent antibody or antigen-binding fragment thereof combines with said second bivalent antibody or antigen binding fragment thereof via said tetramerization means to form a tetameric, bispecific antibody, or tetameric, bispecific antigen binding fragment thereof;
(Dt) permitting expression of bispecific, tetravalent antibodies, or bispecific antigen-binding fragments thereof, from said host cells;
(Et) contacting said bispecific, tetravalent antibodies, or bispecific antigen-binding fragments thereof with an antigen comprising two non-identical epitopes; and
(Ft) recovering polynucleotides from said first library which encode one or more first immunoglobulin subunit polypeptides which, as part of a bispecific, tetravalent antibody, or bispecific antigen-binding fragment thereof, bind to said antigen, wherein said first and second antigen binding domains each bind to one of said two epitopes recited in (Et).

27. The method of claim 26, wherein the polynucleotide of (Bt) encodes a fixed second heavy chain subunit polypeptide which, as part of a defined antigen binding domain, binds to a known epitope, said epitope being identical to one of said two epitopes recited in (Et).

28. The method of claim 26, wherein the polynucleotide of (Bt) is a member of a second library of polynucleotides encoding, through operable association with a transcriptional control region, a plurality of second heavy chain subunit polypeptides which combine with the immunoglobulin subunit polypeptides encoded by the polynucleotides of (At) and (Ct) to form bispecific, tetravalent antibodies, or bispecific antigen-binding fragments thereof.

29. The method of claim 28, wherein the recovery of (Ft) further comprises recovering polynucleotides from said second library which encode a second heavy chain subunit polypeptide, wherein said second heavy chain subunit polypeptide, when combined with a light chain subunit polypeptide encoded by a polynucleotide of (At) or (Ct) forms an antigen binding domain specific for at least one of the two epitopes recited in (Et).

30. The method of claim 26, wherein the polynucleotide of (Ct) encodes a fixed immunoglobulin subunit polypeptide which, as part of an antigen binding domain, binds to one of said two epitopes recited in (Et).

31. The method of claim 30, wherein said fixed immunoglobulin subunit polypeptide is a first heavy chain subunit polypeptide.

32. The method of claim 30, wherein said fixed immunoglobulin subunit polypeptide is a light chain subunit polypeptide.

33. The method of claim 32, wherein said fixed light chain subunit polypeptide combines with the heavy chain subunit polypeptides encoded by the polynucleotides of (At) and (Bt) to form bispecific, tetravalent antibodies, or bispecific antigen-binding fragments thereof.

34. The method of claim 32, wherein the polynucleotide of (Bt) encodes a fixed second heavy chain subunit polypeptide, and wherein said fixed light chain subunit polypeptide combines with said fixed second heavy chain subunit polypeptide to form a defined antigen binding domain which binds to a known epitope, said epitope being identical to one of said two epitopes recited in (Et).

35. The method of claim 32 wherein said light chain subunit polypeptide combines with both said first heavy chain subunit polypeptide and said second heavy chain subunit polypeptide to form two non-identical antigen binding domains, each of which binds to one of said two epitopes recited in (Et).

36. The method of claim 26, wherein the polynucleotide of (Ct) is a member of a third library of polynucleotides encoding, through operable association with a transcriptional control region, a plurality of immunoglobulin subunit polypeptides which combine with the immunoglobulin subunit polypeptides encoded by the polynucleotides of (At) and optionally, (Bt), to form bispecific, tetravalent antibodies, or bispecific antigen-binding fragments thereof.

37. The method of claim 36, wherein the recovery of (Ft) further comprises recovering polynucleotides from said third library which encode an immunoglobulin subunit polypeptide, wherein said immunoglobulin subunit polypeptide, when combined with an immunoglobulin subunit polypeptide encoded by a polynucleotide of (At), and optionally, (Bt), forms an antigen binding domain specific for at least one of the two epitopes recited in (Et).

38. The method of claim 26, further comprising:
(Gt) introducing the polynucleotides recovered in (Ft) into a population of host cells capable of expressing said bispecific, tetravalent antibody, or bispecific antigen-binding fragment thereof;
(Ht) introducing into said host cells polynucleotides of (Bt) or (Ct) which encode immunoglobulin subunit polypeptide types not encoded by the polynucleotides of (Gt);
(It) permitting expression of bispecific, tetravalent antibodies, or bispecific antigen-binding fragments thereof, from said host cells;
(Jt) contacting said bispecific, tetravalent antibodies, or bispecific antigen-binding fragments thereof with the antigen of (Et); and
(Kt) recovering polynucleotides of (Gt) which encode an immunoglobulin subunit polypeptide which, as part of a bispecific, tetravalent antibody, or bispecific antigen-binding fragment thereof, binds to said antigen, wherein said first and second antigen binding domains each bind to one of said two epitopes recited in (Et).

39. The method of claim 38, further comprising repeating steps (Gt)-(Kt) one or more times, thereby enriching for those polynucleotides of (Gt) which encode an immunoglobulin subunit polypeptide which, as part of a bispecific, tetravalent antibody, or bispecific antigen-binding fragment thereof, binds to said antigen, wherein said first and second antigen binding domains each bind to one of said two epitopes recited in (Et).

40. The method of claim 26, further comprising
(Lt) isolating the polynucleotides recovered in (Ft) or (Kt).

41. The method of claim 40, further comprising:
(Mt) introducing into a population of host cells capable of expressing said bispecific, tetravalent antibody, or bispecific antigen-binding fragment thereof polynucleotides of (Bt) or (Ct) which encode immunoglobulin subunit polypeptide types not encoded by said isolated polynucleotides of (Lt);
(Nt) introducing into said host cells said isolated polynucleotides of (Lt);
(Ot) permitting expression of bispecific, tetravalent antibodies, or bispecific antigen-binding fragments thereof, from said host cells;
(Pt) contacting said bispecific, tetravalent antibodies, or bispecific antigen-binding fragments thereof with the antigen of (Et); and
(Qt) recovering polynucleotides of (Mt) which encode an immunoglobulin subunit polypeptide not encoded by said isolated polynucleotides of (Lt) which, as part of a bispecific, tetravalent antibody, or bispecific antigen-binding fragment thereof, binds to said antigen, wherein said first and second antigen binding domains each bind to one of said two epitopes recited in (Et).

42. The method of claim 41, further comprising:
(Rt) introducing the polynucleotides recovered in (Qt) into a population of host cells capable of expressing said bispecific, tetravalent antibody, or bispecific antigen-binding fragment thereof;
(St) introducing into said host cells the isolated polynucleotides of (Lt), and those polynucleotides of (Bt) or (Ct) which encode one or more immunoglobulin subunit polypeptide types not encoded by said recovered polynucleotides of (Qt) or said isolated polynucleotides of (Lt);
(Tt) permitting expression of bispecific, tetravalent antibodies, or bispecific antigen-binding fragments thereof, from said host cells;
(Ut) contacting said bispecific, tetravalent antibodies, or bispecific antigen-binding fragments thereof with the antigen of (Et); and
(Vt) recovering those polynucleotides of (Rt) which encode an immunoglobulin subunit polypeptide which, as part of a bispecific, tetravalent antibody, or bispecific antigen-binding fragment thereof, binds to said antigen, wherein said first and second antigen binding domains each bind to one of said two epitopes recited in (Et).

43. The method of claim 42, further comprising repeating steps (Rt)-(Vt) one or more times, thereby enriching for those polynucleotides of (Rt) which encode an immunoglobulin subunit polypeptide which, as part of a bispecific, tetravalent antibody, or bispecific antigen-binding fragment thereof, binds to said antigen, wherein said first and second antigen binding domains each bind to one of said two epitopes recited in (Et).

44. The method of claim 41, further comprising
(Wt) isolating the polynucleotides recovered in (Qt) or (Vt).

45. The method of claim 44, further comprising:
(Xt) introducing into a population of host cells capable of expressing said bispecific, tetravalent antibody, or bispecific antigen-binding fragment thereof polynucleotides of (Bt) or (Ct) which encode immunoglobulin subunit polypeptide types not encoded by said isolated polynucleotides of (Lt) and (Wt);
(Yt) introducing into said host cells said isolated polynucleotides of (Lt) and (Wt);
(Zt) permitting expression of bispecific, tetravalent antibodies, or bispecific antigen-binding fragments thereof, from said host cells;
(AAt) contacting said bispecific, tetravalent antibodies, or bispecific antigen-binding fragments thereof with the antigen of (Et); and
(BBt) recovering polynucleotides of (Xt) which encode an immunoglobulin subunit polypeptide not encoded by said isolated polynucleotides of (Lt) and (Wt) which, as part of a bispecific, tetravalent antibody, or bispecific antigen-binding fragment thereof, binds to said antigen, wherein said first and second antigen binding domains each bind to one of said two epitopes recited in (Et).

46. The method of claim 45, further comprising:
(CCt) introducing the polynucleotides recovered in (BBt) into a population of host cells capable of expressing said bispecific, tetravalent antibody, or bispecific antigen-binding fragment thereof;
(DDt) introducing into said host cells the isolated polynucleotides of (Lt) and (Wt);
(EEt) permitting expression of bispecific, tetravalent antibodies, or bispecific antigen-binding fragments thereof, from said host cells;
(FFt) contacting said bispecific, tetravalent antibodies, or bispecific antigen-binding fragments thereof with the antigen of (Et); and
(GGt) recovering those polynucleotides of (CCt) which encode an immunoglobulin subunit polypeptide which, as part of a bispecific, tetravalent antibody, or bispecific antigen-binding fragment thereof, binds to said antigen, wherein said first and second antigen-binding domains each bind to one of said two epitopes recited in (Et).

47. The method of claim 46, further comprising repeating steps (CCt)-(GGt) one or more times, thereby enriching for those polynucleotides of (CCt) which encode an immunoglobulin subunit polypeptide which, as part of a bispecific, tetravalent antibody, or bispecific antigen-binding fragment thereof, binds to said antigen, wherein said first and second antigen binding domains each bind to one of said two epitopes recited in (Et).

48. The method of claim 45, further comprising
(HHt) isolating the polynucleotides recovered in (BBt) or (GGt).

49-211. (canceled)

212. A kit for the identification of bispecific, bivalent antibodies, or antigen-binding fragments thereof expressed in a eukaryotic host cell comprising:
(A) a first library of polynucleotides encoding, through operable association with a transcriptional control region, a plurality of immunoglobulin subunit polypeptides or fragments thereof selected from the group consisting of:
(i) first heavy chain subunit polypeptides, each polypeptide comprising
(a) a first heavy chain constant region, said constant region comprising a first heterodimerization domain,
(b) a first heavy chain variable region fused to the N-terminus of said first heavy chain constant region, and
(c) a signal peptide capable of directing secretion or cell surface expression of said heavy chain subunit polypeptide, fused to the N-terminus of said first heavy chain variable region; and
(ii) light chain subunit polypeptides, each polypeptide comprising
(a) a light chain constant region,
(b) a light chain variable region fused to the N-terminus of said light chain constant region, and
(c) a signal peptide capable of directing secretion of said light chain subunit polypeptide, fused to the N-terminus of said light chain variable region,
wherein said first library is constructed in a eukaryotic virus vector;
(B) a second library of polynucleotides encoding, through operable association with a transcriptional control region, a plurality of second heavy chain subunit polypeptides, or fragments thereof each comprising
(a) a second heavy chain constant region, said constant region comprising a second heterodimerization domain, wherein said second heterodimerization domain interacts with said first heterodimerization domain to promote formation of a heavy chain heterodimer,
(b) a second heavy chain variable region fused to the N-terminus of said second heavy chain constant region, and
(c) a signal peptide capable of directing secretion or cell surface expression of said heavy chain subunit polypeptide, fused to the N-terminus of said second heavy chain variable region
wherein said second library is constructed in a eukaryotic virus vector;
(C) a third library of polynucleotides encoding, through operable association with a transcriptional control region, a plurality of immunoglobulin subunit polypeptides, or fragments thereof wherein said immunoglobulin subunit polypeptides are light chains if the immunoglobulin subunit polypeptides of (A) are first heavy chains, and said one or more immunoglobulin subunit polypeptides are first heavy chains if the immunoglobulin subunit polypeptides of (A) are light chains,
wherein said third library is constructed in a eukaryotic virus vector; and
(D) a population of host cells capable of expressing said bispecific, bivalent antibodies or antigen-binding fragments thereof.

213. A kit for the identification of bispecific, tetravalent antibodies, or antigen-binding fragments thereof expressed in a eukaryotic host cell comprising:
(A) a first library of polynucleotides encoding, through operable association with a transcriptional control region, a plurality of immunoglobulin subunit polypeptides, or fragments thereof selected from the group consisting of:
(i) first heavy chain subunit polypeptides, each polypeptide comprising
(a) a first heavy chain constant region, said constant region comprising a means for tetramerization,
(b) a first heavy chain variable region fused to the N-terminus of said first heavy chain constant region, and
(c) a signal peptide capable of directing secretion or cell surface expression of said heavy chain subunit polypeptide, fused to the N-terminus of said first heavy chain variable region; and
(ii) light chain subunit polypeptides, each polypeptide comprising
(a) a light chain constant region,
(b) a light chain variable region fused to the N-terminus of said light chain constant region, and
(c) a signal peptide capable of directing secretion of said light chain subunit polypeptide, fused to the N-terminus of said light chain variable region,
wherein said first library is constructed in a eukaryotic virus vector;
(B) a second library of polynucleotides encoding, through operable association with a transcriptional control region, a plurality of second heavy chain subunit polypeptides, or fragments thereof each comprising
(a) a second heavy chain constant region, said constant region comprising a means for tetramerization,
(b) a second heavy chain variable region fused to the N-terminus of said second heavy chain constant region, and
(c) a signal peptide capable of directing secretion or cell surface expression of said heavy chain subunit polypeptide, fused to the N-terminus of said second heavy chain variable region
wherein said second library is constructed in a eukaryotic virus vector;
(C) a third library of polynucleotides encoding, through operable association with a transcriptional control region, a plurality of immunoglobulin subunit polypeptides, or fragments thereof wherein said immunoglobulin subunit polypeptides are light chains if the immunoglobulin subunit polypeptides of (A) are first heavy chains, and said immunoglobulin subunit polypeptides are first heavy chains if the immunoglobulin subunit polypeptides of (A) are light chains,
wherein said third library is constructed in a eukaryotic virus vector; and
(D) a population of host cells capable of expressing said bispecific, bivalent antibodies or antigen-binding fragments thereof.

214-216. (canceled)
